SEQUENCE LISTING (10 sequences)

SEQ ID NO: 1 - DNA, 21 bp, Homo sapiens
cttgactgcaagatgaaactc

SEQ ID NO: 2 - DNA, 21 bp, Homo sapiens
ctgacaatacttcttggtgtc

SEQ ID NO: 3 - DNA, 20 bp, Homo sapiens
accatgaagaccctcctact

SEQ ID NO: 4 - DNA, 20 bp, Homo sapiens
gaagaggggactcagcaacg

SEQ ID NO: 5 - DNA, 22 bp, Homo sapiens
cagggggcttgctagaactgaa

SEQ ID NO: 6 - DNA, 22 bp, Homo sapiens
aagacggttgtaactccagagg

SEQ ID NO: 7 - DNA, 22 bp, Homo sapiens
cgcgcccggccaaataaaataa

SEQ ID NO: 8 - DNA, 24 bp, Homo sapiens
cagcgacggcagtagcaggagcag

SEQ ID NO: 9 - DNA, 24 bp, Homo sapiens
cagaagaaaggcagttctggattg

SEQ ID NO: 10 - DNA, 23 bp, Homo sapiens
aaagccacatcctggctctgatt

(19) United States
(12) Patent Application Publication
(10) Pub. No.: US 2013/0316973 A1
(43) Pub. Date: Nov. 28, 2013

(54) USE OF LIPID CONJUGATES IN THE TREATMENT OF CANCER

(71) Applicant: YISSUM RESEARCH DEVELOPMENT COMPANY, Jerusalem (IL)

(72) Inventor: Saul YEDGAR, Jerusalem (IL)

(73) Assignee: YISSUM RESEARCH DEVELOPMENT COMPANY, Jerusalem (IL)

(21) Appl. No.: 13/779,005

(22) Filed: Feb. 27, 2013

Related U.S. Application Data
(63) Continuation-in-part of application No. 12/463,792, filed on May 11, 2009, now abandoned.
Application No. 12/463,792 is a continuation-in-part of application No. 11/822,423, filed on Jul. 5, 2007, now Pat. No. 8,304,395.
Application No. 11/822,423 is a continuation-in-part of application No. 10/989,606, filed on Nov. 17, 2004, now Pat. No. 7,811,999.
Application No. 10/989,606 is a continuation-in-part of application No. 10/627,981, filed on Jul. 28, 2003, now Pat. No. 7,101,859.
Application No. 11/822,423 is a continuation-in-part of application No. 10/952,496, filed on Sep. 29, 2004, now Pat. No. 7,393,938.
Application No. 10/627,981 is a continuation-in-part of application No. 09/756,765, filed on Jan. 10, 2001, now Pat. No. 7,034,006.
Application No. 10/952,496 is a continuation-in-part of application No. 09/756,765, filed on Jan. 10, 2001, now Pat. No. 7,034,006.
(60) Provisional application No. 60/174,907, filed on Jan. 10, 2000.
(60) Provisional application No. 60/174,905, filed on Jan. 10, 2000.

(51) Int. Cl. A61K 47/48

(52) U.S. Cl. CPC A61K 47/4823
USPC 514/56; 514/54; 435/375

(57) ABSTRACT

This invention provides for the use of compounds represented by the structure of the general formula (A):

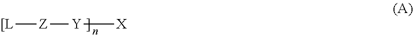

wherein L is a lipid or a phospholipid, Z is either nothing, ethanolamine, serine, inositol, choline, or glycerol, Y is either nothing or a spacer group ranging in length from 2 to 30 atoms, X is a physiologically acceptable monomer, dimer, oligomer, or polymer, wherein X is a glycosaminoglycan; and n is a number from 2 to 1000, wherein any bond between L, Z, Y and X is either an amide or an esteric bond in treating a subject suffering from a disease associated with elevated level of a Matrix Metalloprotease (MMP) such as a malignant cancer.

### CROSS-REFERENCE TO RELATED APPLICATIONS

This application is a continuation-in-part of U.S. application Ser. No. 12/463,792, filed May 11, 2009, which is a continuation-in-part of U.S. application Ser. No. 11/822,423, filed Jul. 5, 2007 which is a continuation-in-part of: (1) U.S. application Ser. No. 10/989,606 filed Nov. 17, 2004, 2001, which is a continuation-in-part of U.S. application Ser. No. 10/627,981, filed Jul. 28, 2003; and (2) U.S. application Ser. No. 10/952,496 filed Sep. 29, 2004; each of which is a continuation-in-part of U.S. application Ser. No. 09/756,765, filed Jan. 10, 2001, which claims priority to U.S. Provisional Application Ser. No. 60/174,907, filed Jan. 10, 2000 and U.S. Provisional Application Ser. No. 60/174,905, filed Jan. 10, 2000. Each and All patent applications referenced above are hereby incorporated by reference.

### FIELD OF THE INVENTION

This invention is directed to lipid-GAG conjugates and phospholipid-GAG conjugates for inhibiting a matrix metalloproteinase.

### BACKGROUND OF THE INVENTION

Matrix metalloproteinases (MMPs), especially MMP-2 and MMP-9, are expressed in most colonic, gastric, and ovarian carcinomas, and they play a key role in their invasiveness.

A major cause of morbidity in patients with cancer is the metastatic spread of tumor cells, governed by a number of processes: invasiveness of tumor cells through the basement membrane, proliferation of the tumor cells in specific sites, and tumor vascularization which is essential for its growth. The major components of the basement membrane, comprising the barrier to the invading tumor cells, are collagen IV, laminin and heparane sulfate proteoglycans. The degradation of extracellular matrix (ECM) in mammalian cells is regulated by a family of MMPs, including collagenases, gelatinases, stromelysins and membrane type MMPs. The passage of tumor cells through the basement membrane begins with the binding of the cell to laminin and subsequent activation of a protease cascade, leading to the production of active MMPs from pre-activated MMP forms or pre-<<Ps. These enzymes specifically degrade the major structural element in the ECM: collagen IV. The movement of cells across the basement membrane may occur in response to specific chemotactic and motility factors produced by the host tissue.

MMP production and cancer cell invasiveness have been shown to require the involvement of prostaglandins (PGs) and leukotrienes (LTs) produced via the cyclooxygenases (COX) and lipoxygenases (LOX) pathways. Both PGs and LTs are involved in the development of several types of cancer in humans including: colon, breast, gastric and hepatocellular carcinomas. Different eicosanoids have been shown to facilitate the invasiveness of tumor cells, angiogenesis and tumor vascularization.

Lipid-conjugates having a pharmacological activity of inhibiting the enzyme phospholipase A2 (PLA2, EC 3.1.1.4) are known in the prior art. Phospholipase A2 catalyzes the breakdown of phospholipids at the sn-2 position to produce a fatty acid and a lysophospholipid. The activity of this enzyme has been correlated with various cell functions, particularly with the production of lipid mediators such as eicosanoid production (prostaglandins, thromboxanes and leukotrienes), platelet activating factor and lysophospholipids. Since their inception, lipid-conjugates have been subjected to intensive laboratory investigation in order to obtain a wider scope of protection of cells and organisms from injurious agents and pathogenic processes.

### SUMMARY OF THE INVENTION

In one embodiment, provided a method for treating a subject afflicted with a disease in which increased production of a matrix metalloprotease (MMP) is associated with said disease, comprising the step of administering to said subject a composition comprising a compound represented by the structure of the general formula (A):

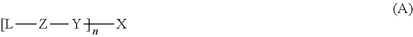

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein X is a glycosaminoglycan; and
n is a number from 2 to 1000;
wherein any bond between L, Z, Y and X is either an amide or an esteric bond, thereby treating a subject afflicted with a disease in which increased production of MMP is implicated.

In another embodiment, further provided is a method of treating a subject afflicted with a metastatic cancer, comprising the step of administering to said subject a composition comprising a compound represented by the structure of the general formula (A):

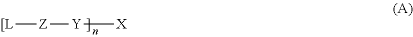

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein X is a glycosaminoglycan; and
n is a number from 2 to 1000; wherein any bond between L, Z, Y and X is either an amide or an esteric bond, thereby treating a subject afflicted with a metastatic cancer.

In another embodiment, further provided is a method of inhibiting the production of a matrix metalloprotease (MMP) in a cell, comprising contacting said cell with a composition comprising a compound represented by the structure of the general formula (A):

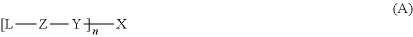

wherein
L is a lipid or a phospholipid;
Z is either nothing, ethanolamine, serine, inositol, choline, or glycerol:
Y is either nothing or a spacer group ranging in length from 2 to 30 atoms;
X is a physiologically acceptable monomer, dimer, oligomer, or polymer, wherein X is a glycosaminoglycan; and
n is a number from 2 to 1000; wherein any bond between L, Z, Y and X is either an amide or an esteric bond, thereby inhibiting invasiveness of a cancer cell.

In another embodiment, further provided is a method of treating a subject afflicted with melanoma, comprising the step of administering to the subject a composition comprising a compound represented by the structure of the general formula (A):

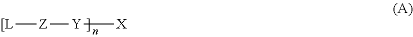

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein X is a glycosaminoglycan; and
n is a number from 2 to 1000; wherein any bond between L, Z, Y and X is either an amide or an esteric bond, thereby treating a subject afflicted with melanoma.

In another embodiment, further provided is a method of inhibiting invasiveness of a cancer cell, comprising the step of contacting said cell with a composition comprising a compound represented by the structure of the general formula (A):

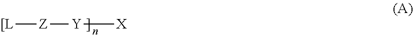

wherein
L is a lipid or a phospholipid;
Z is either nothing, ethanolamine, serine, inositol, choline, or glycerol:
Y is either nothing or a spacer group ranging in length from 2 to 30 atoms;
X is a physiologically acceptable monomer, dimer, oligomer, or polymer, wherein X is a glycosaminoglycan; and
n is a number from 2 to 1000; wherein any bond between L, Z, Y and X is either an amide or an esteric bond, thereby inhibiting invasiveness of a cancer cell.

In another embodiment, further provided is a method of inhibiting a collagenolytic activity of a cell, comprising the step of contacting said cell with a composition comprising a compound represented by the structure of the general formula (A):

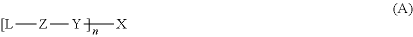

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein X is a glycosaminoglycan; and
n is a number from 2 to 1000; wherein any bond between L, Z, Y and X is either an amide or an esteric bond, thereby inhibiting a collagenolytic activity of a Matrix metalloproteinase.

In another embodiment, further provided is a method A method of inhibiting the production of a Matrix Metalloproteinase (MMP) in a cancer cell, comprising the step of contacting said cell with a composition comprising a compound represented by the structure of the general formula (A):

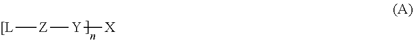

wherein
L is a lipid or a phospholipid,
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein X is a glycosaminoglycan; and
n is a number from 2 to 1000; wherein any bond between L, Z, Y and X is either an amide or an esteric bond, thereby inhibiting the production of a Matrix Metalloproteinase (MMP) in a cancer cell.

In one embodiment, further provided is a method of treating a subject afflicted with lung cancer, comprising the step of administering to the subject a composition comprising a compound represented by the structure of the general formula (I):

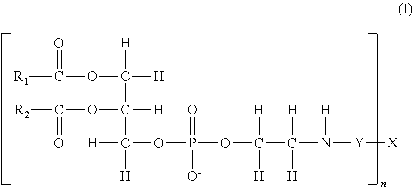

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan alginate or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

In one embodiment, further provided is a method of attenuating invasiveness of a cancer cell, comprising the step of subjecting the cancer cell to a composition comprising a compound represented by the structure of the general formula (I):

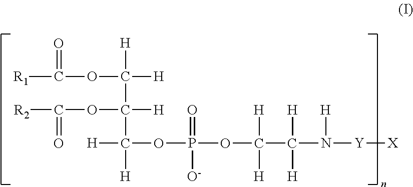

wherein
is R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan alginate or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

In one embodiment, further provided is a method of inhibiting proliferation of an endothelial cell or inhibiting capillary formation, comprising the step of subjecting the endothelial cell to a composition comprising a compound represented by the structure of the general formula (I):

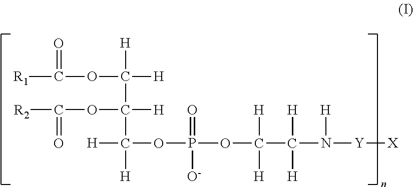

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan alginate or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

According to one embodiment, n is a number from 1 to 1,000. In another embodiment, n is a number from 2 to 1,000. In another embodiment, n is a number from 2 to 500. In another embodiment, n is a number from 1 to 500. In another embodiment, n is a number from 1 to 100. In another embodiment, n is a number from 2 to 1000. In another embodiment, n is a number from 2 to 100. In another embodiment, n is a number from 2 to 200. In another embodiment, n is a number from 3 to 300. In another embodiment, n is a number from 10 to 400. In another embodiment, n is a number from 50 to 500. In another embodiment, n is a number from 100 to 300. In another embodiment, n is a number from 300 to 500. In another embodiment, n is a number from 500 to 800. In another embodiment, n is a number from 500 to 1000.

According to one embodiment, the glycosaminoglycan is selected from the group consisting of hyaluronic acid, heparin, heparan sulfate, chondrotin sulfate, keratan, keratan sulfate, dermatan sulfate or a derivative thereof.

The phosphatidylethanolamine may be a myristoyl or palmitoyl phosphatidylethanolamine, or further dipalmitoyl phosphatidylethanolamine, or dimyristoyl phosphatidylethanolamine.

### BRIEF DESCRIPTION OF DRAWINGS

FIG. 1 is a bar graph showing the inhibitory effect of ExPLI (lipid conjugates) on the invasion capacity of HT-1080 cell. HT-1080 cells were treated with the ExPLI HyPE, composed of Hyaluronic acid (HA) conjugated PE and with HA, at the indicated concentrations, for 24 h, than washed and placed on a Matrigel membrane. Cell invasion through the Matrigel was determined. Each datum is Mean and SD for 3 replications (a, b, P<0.05).

FIG. 2 upper panel is a micrograph of a gel followed by a bar graph (lower panel). This graph demonstrates the inhibitory effect of HyPE on MMP-2 and MMP-9 activity. HT-1080 were incubated for 24 h with either HyPE or HA. The cultured medium was then collected and subjected to determination of MMP-2 (72 kDa) and MMP-9 (96 kDa) content and their collagenolytic activity, using zymography as described in Materials and methods. Each datum is Mean and SD for 4 replications (*, P<0.05, **, P<0.01).

FIG. 3 are bar graphs showing the inhibitory effect of ExPLI ((lipid conjugates) on PLA2 activity in HT-1080 cells as evidenced by the release of Arachidonic acid (AA) (lower panel) or Oleic acid (upper panel) from HT-1080 cells. HT-1080 cells were metabolically labeled by overnight incubation with either 3H-arachidonic acid or 3H-oleic acid, then washed and the release of the labeled AA or OA into the culture medium during the indicated time, in the absence (clear bar) or presence (black bar) of HyPE was measured. Each datum is Mean and SD for 3 replications. (*, P<0.05).

FIG. 4 is a gel micrograph showing expression of PLA2s and PLA2 receptor by HT-1080 cells. mRNA expression of the indicated PLA2 was determined by RT-PCR using primers as described in the experimental section. The figures depicts RT-PCR of cPLA2α (988 bp); sPLA2 Types V, IIA and IB (329 bp, 449 bp and 243 bp respectively); and M-Type sPLA2 receptor (Rec, 565 bp).

FIG. 5 is a bar graph showing the effect of heat inactivation on lipolytic activity of porcine pancreatic and crotalus atrox sPLA2s (ppPLA2 and caPLA2, respectively). The enzymes were denatured by heating at 95° C. for 15 min, and their lipolytic capacity was determined by their ability to hydrolyze 4N3OBA, as in the experimental section. Each datum is Mean and SD for 3 replications. (*, P<0.05).

FIG. 6 are graphs followed by gel micrographs demonstrating the effect of heat inactivation of ppPLA2 on its ability to induce MMP activity/production. HT-1080 cells were treated with either intact or denatured (d) ppPLA2 for 6 h, and the activity of MMP-9 (A) and MMP-2 (B) activity was determined by zymography. Each datum is Mean and SD for 3 replications (*, P<0.01).

FIG. 7 presents cPLA2 phosphorylation by ppPLA2 and its suppression by heat inactivation or sPLA2 inhibitor. HT-1080 cells were treated with ppPLA2 in the absence or presence of HyPE, or with denatured ppPLA2 for 15 min prior to protein isolation. The extent of cPLA2 phosphorylation was determined by Western blot analysis with specific antibodies directed against cPLA2 phosphorylated on Ser505 and with specific antibody directed against the total (phosphorylated and non-phosphorylated) cPLA2. Each datum is Mean and SD for 2 replications (a, b, P<0.05).

FIG. 8 is a bar graph showing the effect of HyPE on the transcription of sPLA2 Types IIA and IB by HT-1080 cells. HT-1080 cells were treated for 24 h in the absence or presence of HyPE (10 microM) prior to RNA extraction. The transcription of sPLA2-IB and IIA in these cells were analyzed by RT-PCR using the primers as described in the experimental section. Sample loading was verified by 28s expression. Each datum is Mean and SD for 3 replications (*, P<0.05).

FIG. 9 presents a schematic describing the cascade involved in sPLA2-IB-induced MMP activity. sPLA2 binds to a membranal receptor and activates intracellular cPLA2. cPLA2 in its turn, releases AA that is converted into eicosanoids. SPLA2-induced eicosanoids eventually induce MMP expression.

FIG. 10 is a bar graph showing the effect of Lipid-conjugates on invasiveness of human fibrosarcoma cells (A); FIG. 10(B) is a bar graph showing the inhibitory effect of dipalmitoyl phosphatidylethanolamine hyaluronic acid (HyPE) and dimyristoyl phosphatidylethanolamine hyurolonic acid (HyDMPE) on invasiveness of human fibrosarcoma (HT-1080) cells.

FIG. 10C: Lipid-conjugates inhibit proliferation of bovine aortic endothelial cells (EC).

FIG. 10D: HyPE inhibits proliferation of human bone marrow endothelial cells (HBMEC) induced by growth factors.

FIG. 10E: Lipid-conjugates inhibit growth factor-induced capillary formation by HBMEC in fibringel.

FIG. 10F: HyPE inhibits capillary tube formation in a three-dimensional fibrin gel in conditions where HBMEC-coated beads were firstly incubated with the growth factors for a period of 3 h, and washed and then incorporated into the fibrin gel without or with HyPE. Row A: bFGF (25 ng/ml); row B: VEGF (20 ng/ml); row C: OSM (2.5 ng/ml). Column 1: without HyPE; column 2: with HyPE (20 μM).

FIG. 10G: Lipid-conjugates suppress mouse lung metastases formation induced by mouse melanoma cells.

FIG. 11 is a graph showing that Lipid-conjugates inhibit secretion of collagenase IV/activity of MMP-2 in human fibrosarcoma cells.

FIG. 12 is a graph showing that HyPE inhibits hyaluronic acid degradation by hyaluronidase.

FIGS. 13A and 13B are bar graphs showing that Lipid-conjugates inhibit the activity of exogenous heparinase.

FIG. 14 is a bar graph showing that HyPE inhibits bovine aortic smooth muscle cell (SMC) proliferation.

FIG. 15 is a bar graph showing that HyPE inhibits proliferation of bovine aortic SMCs, stimulated with thrombin (48 hours).

FIG. 16 depicts an NMR spectrum of a hyaluronic acid-phosphatidylethanolamine conjugate (HyPE) prepared according to Example 10.

FIG. 17 is an HPLC chromatogram of HyPE prepared according to Example 10.

FIG. 18 depicts a conceptual diagram of the reaction vessel features required to practice the methods of this invention.

FIG. 19: depicts a chromatogram of the HyPE reaction from Example 11 after 6 hours.

FIG. 20: depicts the GPC analysis of final HyPE isolated from Example 11.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In one embodiment, this invention provides a method of inhibiting a Matrix metalloproteinase (MMP). In another embodiment, the invention provides a method of inhibiting MMP in a subject. In another embodiment, the invention provides a method of inhibiting MMP in a cancerous cell. In another embodiment, the invention provides a method of inhibiting MMP in a cancerous cell in a subject. In another embodiment, the invention provides a method of inhibiting MMP in a metastatic cell. In another embodiment, the invention provides a method of inhibiting MMP in a metastatic cell in a subject. In another embodiment, the invention provides a method of inhibiting MMP in a tumor cell. In another embodiment, the invention provides a method of inhibiting MMP in a tumor cell in a subject.

In another embodiment, the invention provides a method based on the use of the compounds of invention as MMP inhibitors. In another embodiment, the invention provides a method based on the use of the compounds of invention as MMP 2 inhibitors. In another embodiment, the invention provides a method based on the use of the compounds of invention as MMP 9 inhibitors.

In another embodiment, the invention provides a method of inhibiting the development of a primary tumor or a lesion to a metastatic cancer.

In another embodiment, this invention provides a method for treating a subject afflicted with a disease or a pathology characterized by elevated MMP levels via administration of a compound comprising a lipid or a phospholipid bonded, directly or via a spacer group, to a physiologically acceptable monomer, dimer, oligomer, or polymer. In another embodiment, this invention provides a method for treating a subject afflicted with a disease or a pathology mediated by elevated MMP levels via administration of a compound comprising a lipid or a phospholipid bonded, directly or via a spacer group, to a physiologically acceptable monomer, dimer, oligomer, or polymer. In another embodiment, this invention provides a method for treating a subject afflicted with a disease or a pathology induced by elevated MMP levels via administration of a compound comprising a lipid or a phospholipid bonded, directly or via a spacer group, to a physiologically acceptable monomer, dimer, oligomer, or polymer. In another embodiment, this invention provides that a compound comprising a lipid or a phospholipid bonded, directly or via a spacer group, to a physiologically acceptable monomer, dimer, oligomer, or polymer is a MMP inhibitor. In another embodiment, this invention provides a method for treating a subject afflicted with a malignant tumor via administration of a compound comprising a lipid or a phospholipid bonded, directly or via a spacer group, to a physiologically acceptable monomer, dimer, oligomer, or polymer. In another embodiment, this invention provides a method for inhibiting cancer spread in a subject via administration of a compound comprising a lipid or a phospholipid bonded, directly or via a spacer group, by an amide or an ester bond to a glycosaminoglycan.

In another embodiment, this invention provides administration of the conjugates for the treatment of diseases which require controlling phospholipase A2 activities, controlling the production and/or action of lipid mediators, amelioration of damage to cell surface by glycosaminoglycans (GAG) and proteoglycans, controlling the production of oxygen radicals and nitric oxide, protection of lipoproteins from damaging agents, anti-oxidant therapy; anti-endotoxin therapy; controlling of cytokine, chemokine and interleukin production; controlling the proliferation of cells, controlling of angiogenesis and organ vascularization; inhibition of invasion-promoting enzymes, inhibition of a MMP, controlling of cell invasion, controlling of leukocyte activation, adhesion and extravasation, amelioration of ischemia/reperfusion injury, inhibition of lymphocyte activation, controlling of blood vessel and airway contraction, protection of blood brain barrier, controlling of neurotransmitter production and action or extracorporeal tissue preservation.

In another embodiment of the invention, the lipid-conjugates described are used in a process for manufacture of a composition for the treatment of diseases which requires controlling phospholipase A2 activities, controlling the production and/or action of lipid mediators, amelioration of damage to cell surface by glycosaminoglycans (GAG) and proteoglycans, controlling of cytokine, chemokine and interleukine production; controlling the proliferation of cells, inhibiting MMP activity/production (expression and/or transcription) controlling of angiogenesis and organ vascularization; inhibition of invasion-promoting enzymes, controlling of cell invasion, controlling of white cell activation, adhesion and extravasation.

Metastasis, the spread of cancer cells to ectopic sites, is frequently a vasculature dependent process as well, often referred to as hematogenous spread. The physiological barrier imposed by the blood vessel wall, comprised from elements such as endothelial cells and basement membrane substance, is normally highly selective to the passage of cells. However, metastatic cells abrogate this barrier, employing a variety of mechanisms, some of which have been established in the scientific literature. For example, such abnormal cells produce hydrolytic enzymes which degrade the extracellular matrix and associated components of the vascular barrier, such as collagenase, heparinase, and hyaluronidase. Thus a critical factor in the metastatic process is the ability of cancer cells to intrude through or permeate the wall of the blood vessel lumen, thus arriving to invade a new tissue site after travel through the circulation. In another embodiment, a MMP inhibitor as described herein inhibits the intruding capacity of cells. In another embodiment, a MMP inhibitor as described herein inhibits the intruding capacity of metastatic cells. In another embodiment, a MMP inhibitor as described herein inhibits the intruding capacity of tumor cells.

In other embodiments, the lipid-conjugates provide cytoprotective effects to an organism suffering from a disease, where pathophysiological mechanisms of tissue damage may comprise oxidation insult giving rise to membrane fragility; hyperproliferation behavior of cells giving rise to stenotic plaque formation in vascular tissue, angiogenesis and benign or malignant cancer disease, or psoriasis; aberrant cell migration giving rise to brain injury or tumor cell metastases; excessive expression of chemokines and cytokines associated with central nervous system (CNS) insult, sepsis, ARDS, or immunological disease; cell membrane damage giving rise to CNS insult, CVS disease, or hemolysis; peroxidation of blood proteins and cell membranes giving rise to atherosclerosis or reperfusion injury; excessive nitric oxide production giving rise to CNS insult, reperfusion injury, and septic shock; interaction with major histocompatability antigens (MHC) associated with autoimmune diseases alloimmune syndromes, such as transplant rejection, or combinations thereof.

In another embodiment, the treatment requires protection of lipoproteins from damaging agents. In another embodiment, the treatment requires controlling the proliferation of cells. In another embodiment, the treatment requires controlling of angiogenesis and organ vascularization. In another embodiment, the treatment requires inhibition of invasion-promoting enzymes. In another embodiment, the treatment requires controlling of cell invasion. In another embodiment, the invading cells are white blood cells. In another embodiment, the invading cells are cancer cells. In another embodiment, the treatment requires controlling of white cell activation, adhesion or extravasation. In another embodiment, the treatment requires amelioration of ischemia or reperfusion injury. In another embodiment, the treatment requires inhibition of lymphocyte activation. In another embodiment, the treatment requires protection of blood brain barrier. In another embodiment, the treatment requires control of neurotransmitter production and action. In another embodiment, the treatment requires controlling of blood vessel and airway contraction. In another embodiment, the treatment requires extracorporeal tissue preservation.

In one embodiment, the invention provides a method of treating a subject afflicted with a disease, wherein the treatment of the disease requires controlling phospholipase A2 activities; controlling the production and/or action of lipid mediators, such as eicosanoids, platelet activating factor (PAF) and lyso-phospholipids; amelioration of damage to cell surface glycosaminoglycans (GAG) and proteoglycans; controlling the production of oxygen radicals and nitric oxide; protection of cells, tissues, and plasma lipoproteins from damaging agents, such as reactive oxygen species (ROS) and phospholipases; anti-oxidant therapy; anti-endotoxin therapy; controlling of cytokine, chemokine and interleukine production; controlling the proliferation of cells, including smooth muscle cells, endothelial cells and skin fibroblasts; controlling of angiogenesis and organ vascularization; inhibition of invasion-promoting enzymes, such as collagenase, heparinase, heparanase and hyaluronidase; controlling of cell invasion; controlling of white cell activation, adhesion and extravasation; amelioration of ischemia/reperfusion injury, inhibition of lymphocyte activation; controlling of blood vessel and airway contraction; protection of blood brain barrier; controlling of neurotransmitter (e.g., dopamine) production and action (e.g., acethylcholine); extracorporeal tissue preservation or any combination thereof.

### Compounds

In one embodiment, reference to a compound for use in a method of the present invention refers to one comprising a lipid or phospholipid moiety bound to a physiologically acceptable monomer, dimer, oligomer, or polymer. In one embodiment, the compounds for use in the present invention are referred to as “Lipid-conjugates.” In another embodiment, reference to a MMP inhibitor for use in a method of the present invention refers to one comprising a lipid or phospholipid moiety bound to a physiologically acceptable monomer, dimer, oligomer, or polymer. In one embodiment, the compounds for use in the present invention are referred to as “Lipid-conjugates.” In another embodiment, compounds for use in the present invention are described by the general formula:

[phosphatidylethanolamine-Y]n-X
[phosphatidylserine-Y]n-X
[phosphatidylcholine-Y]n-X
[phosphatidylinositol-Y]n-X
[phosphatidylglycerol-Y]n-X
[phosphatidic acid-Y]n-X
[lyso-phospholipid-Y]n-X
[diacyl-glycerol-Y]n-X
[monoacyl-glycerol-Y]n-X
[sphingomyelin-Y]n-X
[sphingosine-Y]n-X
[ceramide-Y]n-X
wherein
Y is either nothing or a spacer group ranging in length from 2 to 30 atoms; and
X is a physiologically acceptable monomer, dimer, oligomer or polymer: and
n is the number of lipid molecules bound to a molecule of X, wherein n is a number from 1 to 1000 or a number from 2 to 1000.

In one embodiment, the invention provides low-molecular weight Lipid-conjugates, which possess pharmacological activity, which are characterized by the general formula described hereinabove.

In one embodiment of the invention, the physiologically acceptable monomer is salicylate. In another embodiment, the physiologically acceptable monomer is salicylic acid. In another embodiment, the physiologically acceptable monomer is acetyl salicylic acid. In another embodiment, the physiologically acceptable monomer is aspirin. In another embodiment, the physiologically acceptable monomer is a monosaccharide. In another embodiment, the physiologically acceptable monomer is lactobionic acid. In another embodiment, the physiologically acceptable monomer is glucoronic acid. In another embodiment, the physiologically acceptable monomer is maltose. In another embodiment, the physiologically acceptable monomer is an amino acid. In another embodiment, the physiologically acceptable monomer is glycine. In another embodiment, the physiologically acceptable monomer is a carboxylic acid. In another embodiment, the physiologically acceptable monomer is an acetic acid. In another embodiment, the physiologically acceptable monomer is a butyric acid. In another embodiment, the physiologically acceptable monomer is a dicarboxylic acid. In another embodiment, the physiologically acceptable monomer is a fatty acid. In another embodiment, the physiologically acceptable monomer is a dicarboxylic fatty acid. In another embodiment, the physiologically acceptable monomer is a glutaric acid. In another embodiment, the physiologically acceptable monomer is succinic acid. In another embodiment, the physiologically acceptable monomer is dodecanoic acid. In another embodiment, the physiologically acceptable monomer is didodecanoic acid. In another embodiment, the physiologically acceptable monomer is bile acid. In another embodiment, the physiologically acceptable monomer is cholic acid. In another embodiment, the physiologically acceptable monomer is cholesterylhemisuccinate.

In one embodiment of the invention, the physiologically acceptable dimer or oligomer is a dipeptide. In another embodiment, the physiologically acceptable dimer or oligomer is a disaccharide. In another embodiment, the physiologically acceptable dimer or oligomer is a trisaccharide. In another embodiment, the physiologically acceptable dimer or oligomer is an oligosaccharide. In another embodiment, the physiologically acceptable dimer or oligomer is an oligopeptide. In another embodiment, the physiologically acceptable dimer or oligomer is a glycoprotein mixture. In another embodiment, the physiologically acceptable dimer or oligomer is a di- or trisaccharide monomer unit of a polysaccharide. In another embodiment, the physiologically acceptable dimer or oligomer is a di- or trisaccharide monomer unit of a polypyranose. In another embodiment, the physiologically acceptable dimer or oligomer is a di- or trisaccharide monomer unit of a glycosaminogylcan. In another embodiment, the physiologically acceptable dimer or oligomer is a di- or trisaccharide monomer unit of a hyaluronic acid. In another embodiment, the physiologically acceptable dimer or oligomer is a di- or trisaccharide monomer unit of a heparin. In another embodiment, the physiologically acceptable dimer or oligomer is a di- or trisaccharide monomer unit of a heparan sulfate. In another embodiment, the physiologically acceptable dimer or oligomer is a di- or trisaccharide monomer unit of a keratin. In another embodiment, the physiologically acceptable dimer or oligomer is a di- or trisaccharide monomer unit of a keratan sulfate. In another embodiment, the physiologically acceptable dimer or oligomer is a di- or trisaccharide monomer unit of a chondroitin. In another embodiment, the chondroitin is chondoitin sulfate. In another embodiment, the chondroitin is chondoitin-4-sulfate. In another embodiment, the chondroitin is chondoitin-6-sulfate. In another embodiment, the physiologically acceptable dimer or oligomer is a di- or trisaccharide monomer unit of a dermatin. In another embodiment, the physiologically acceptable dimer or oligomer is a di- or trisaccharide monomer unit of a dermatan sulfate. In another embodiment, the physiologically acceptable dimer or oligomer is dextran. In another embodiment, the physiologically acceptable dimer or oligomer is polygeline (‘Haemaccel’). In another embodiment, the physiologically acceptable dimer or oligomer is alginate. In another embodiment, the physiologically acceptable dimer or oligomer is hydroxyethyl starch (Hetastarch). In another embodiment, the physiologically acceptable dimer or oligomer is ethylene glycol. In another embodiment, the physiologically acceptable dimer or oligomer is carboxylated ethylene glycol.

In one embodiment, the physiologically acceptable polymer is a polysaccharide. In another embodiment, the physiologically acceptable polymer is a homo-polysaccharide. In another embodiment, the physiologically acceptable polymer is a hetero-polysaccharide. In another embodiment, the physiologically acceptable polymer is a polypyranose. In another embodiment of the invention, the physiologically acceptable polymer is a glycosaminoglycan. In another embodiment, the physiologically acceptable polymer is hyaluronic acid. In another embodiment, the physiologically acceptable polymer is heparin. In another embodiment, the physiologically acceptable polymer is heparan sulfate. In another embodiment, the physiologically acceptable polymer is chondroitin. In another embodiment, the chondroitin is chondoitin-4-sulfate. In another embodiment, the chondroitin is chondoitin-6-sulfate. In another embodiment, the physiologically acceptable polymer is keratin. In another embodiment, the physiologically acceptable polymer is keratan sulfate. In another embodiment, the physiologically acceptable polymer is dermatin. In another embodiment, the physiologically acceptable polymer is dermatan sulfate. In another embodiment, the physiologically acceptable polymer is carboxymethylcellulose. In another embodiment, the physiologically acceptable polymer is dextran. In another embodiment, the physiologically acceptable polymer is polygeline (‘Haemaccel’). In another embodiment, the physiologically acceptable polymer is alginate. In another embodiment, the physiologically acceptable polymer is hydroxyethyl starch (‘Hetastarch’). In another embodiment, the physiologically acceptable polymer is polyethylene glycol. In another embodiment, the physiologically acceptable polymer is polycarboxylated polyethylene glycol. In another embodiment, the physiologically acceptable polymer is a peptide. In another embodiment, the physiologically acceptable polymer is an oligopeptide. In another embodiment, the physiologically acceptable polymer is a polyglycan. In another embodiment, the physiologically acceptable polymer is a protein. In another embodiment, the physiologically acceptable polymer is a glycoprotein mixture.

The following terms may be used according to certain embodiments of the invention: phosphatidylethanolamine (PE) linked to carboxymethylcellulose (referred to as CMPE), to hyaluronic acid (referred to as HYPE), to heparin (referred to as HepPE), to chondroitin sulfate A (referred to as CSAPE), to Polygeline (haemaccel) (referred to HemPE) or to hydroxyethylstarch (referred to as HesPE). Phosphatidylserine (PS) and other phospholipids linked conjugates may be named similarly.

In one embodiment, examples of polymers which can be employed as the conjugated moiety for producing Lipid-conjugates for use in the methods of this invention may be physiologically acceptable polymers, including water-dispersible or -soluble polymers of various molecular weights and diverse chemical types, mainly natural and synthetic polymers, such as glycosaminoglycans as described hereinabove, plasma expanders, including polygeline (“Haemaccel”, degraded gelatin polypeptide cross-linked via urea bridges, produced by “Behring”), “hydroxyethylstarch” (Hetastarch, HES) and extrans, food and drug additives, soluble cellulose derivatives (e.g., methylcellulose, carboxymethylcellulose), polyaminoacids, hydrocarbon polymers (e.g., polyethylene), polystyrenes, polyesters, polyamides, polyethylene oxides (e.g. polyethyleneglycols, polycarboxyethyleneglycols, polycarboxylated polyethyleneglycols), polyvinnylpyrrolidones, polysaccharides, polypyranoses, alginates, assimilable gums (e.g., xanthan gum), peptides, injectable blood proteins (e.g., serum albumin), cyclodextrin, and derivatives thereof.

In one embodiment of the invention, the lipid or phospholipid moiety is phosphatidic acid. In another embodiment, lipid or phospholipid moiety is an acyl glycerol. In another embodiment, lipid or phospholipid moiety is monoacylglycerol. In another embodiment, lipid or phospholipid moiety is diacylglycerol. In another embodiment, lipid or phospholipid moiety is triacylglycerol. In another embodiment, lipid or phospholipid moiety is sphingosine. In another embodiment, lipid or phospholipid moiety is sphingomyelin. In another embodiment, lipid or phospholipid moiety is ceramide. In another embodiment, lipid or phospholipid moiety is phosphatidylethanolamine. In another embodiment, lipid or phospholipid moiety is phosphatidylserine. In another embodiment, lipid or phospholipid moiety is phosphatidylcholine. In another embodiment, lipid or phospholipid moiety is phosphatidylinositol. In another embodiment, lipid or phospholipid moiety is phosphatidylglycerol. In another embodiment, lipid or phospholipid moiety is an ether or alkyl phospholipid derivative thereof.

In one embodiment, the set of compounds comprising phosphatidylethanolamine covalently bound to a physiologically acceptable monomer, dimmer, oligomer, or polymer, is referred to herein as the PE-conjugates. In one embodiment, the phosphatidylethanolamine moiety is dipalmitoyl phosphatidylethanolamine. In another embodiment, the phosphatidylethanolamine moiety is dimyristoyl phosphatidylethanolamine. In another embodiment, related derivatives, in which either phosphatidylserine, phosphatidylcholine, phosphatidylinositol, phosphatidic acid or phosphatidylglycerol are employed in lieu of phosphatidylethanolamine as the lipid moiety provide equivalent therapeutic results, based upon the biological experiments described below for the Lipid-conjugates and the structural similarities shared by these compounds.

As defined by the structural formulae provided herein for the Lipid-conjugates or phospholipids-conjugates, these compounds may contain between one to one thousand lipid or phospholipid moieties bound to a single physiologically acceptable polymer molecule. In one embodiment of this invention, n is a number from 1 to 1000. In another embodiment, n is a number from 2 to 500. In another embodiment, n is a number from 1 to 500. In another embodiment, n is a number from 1 to 100. In another embodiment, n is a number from 2 to 1000. In another embodiment, n is a number from 2 to 100. In another embodiment, n is a number from 2 to 200. In another embodiment, n is a number from 3 to 300. In another embodiment, n is a number from 10 to 400. In another embodiment, n is a number from 50 to 500. In another embodiment, n is a number from 100 to 300. In another embodiment, n is a number from 300 to 500. In another embodiment, n is a number from 500 to 800. In another embodiment, n is a number from 500 to 1000.

In one embodiment of the invention, when the conjugated moiety is a polymer, the ratio of lipid moieties covalently bound may range from one to one thousand lipid or phospholipids (PL) residues per polymer molecule, depending upon the nature of the polymer and the reaction conditions employed. For example, the relative quantities of the starting materials, or the extent of the reaction time, may be modified in order to obtain Lipid-conjugate or Phospholipid (PL)-conjugate products with either high or low ratios of lipid residues per polymer, as desired.

In the methods, according to embodiments of the invention, the Lipid-conjugates or Phospholipid-conjugate administered to a subject are comprised of at least one lipid or phospholipid moiety covalently bound through an atom of the polar head group to a monomeric or polymeric moiety (referred to herein as the conjugated moiety) of either low or high molecular weight. In one embodiment, the conjugated moiety is conjugated to the lipid, phospholipid, or spacer via an ester bond. In another embodiment, the conjugated moiety is conjugated to the lipid, phospholipid, or spacer via an amide bond.

When desired, an optional bridging moiety can be used to link the lipid or phospholipid moiety to the monomer or polymeric moiety. The composition of some phospholipid-conjugates of high molecular weight, and associated analogues, are the subject of U.S. Pat. No. 5,064,817, which is incorporated herein in its entirety by reference.

In one embodiment, the term “moiety” means a chemical entity otherwise corresponding to a chemical compound, which has a valence satisfied by a covalent bond.

In some cases, according to embodiments of the invention, the monomer or polymer chosen for preparation of the Lipid-conjugate or Phospholipid-conjugate may in itself have selected biological properties. For example, both heparin and hyaluronic acid are materials with known physiological functions. In the present invention, however, the Lipid-conjugates or Phospholipid-conjugate formed from these substances as starting materials display a new and wider set of pharmaceutical activities than would be predicted from administration of either heparin or hyaluronic acid which have not been bound by covalent linkage to a phospholipid. It can be shown, by standard comparative experiments that phosphatidylethanolamine (PE) linked to hyaluronic acid (Compound XXII), to heparin (Compound XXIV), to chondroitin sulfate A (Compound XXV), to carboxymethylcellulose (Compound XXVI), to Polygeline (haemaccel) (Compound XXVII), or to hydroxyethylstarch (Compound XXVIII), are far superior in terms of potency and range of useful pharmaceutical activity to the free conjugates (the polymers above and the like). In fact, these latter substances are, in general, not considered useful in methods for inhibiting MMP activity or production in a cell. Thus, the combination of a phospholipid such as phosphatidylethanolamine, or related phospholipids which differ with regard to the polar head group, such as phosphatidylserine (PS), phosphatidylcholine (PC), phosphatidylinositol (PI), and phosphatidylglycerol (PG), results in the formation of a compound which has novel pharmacological properties when compared to the starting materials alone. In the cases described herein, the diversity of biological activities and the effectiveness in disease exhibited by the compounds far exceed the properties anticipated by use of the starting materials themselves, when administered alone or in combination.

The biologically active Lipid-conjugates or Phospholipid-conjugates described herein can have a wide range of molecular weights, e.g., above 50,000 (up to a few hundred thousands) when it is desirable to retain the conjugates in the vascular system and below 50,000 when targeting to extravascular systems is desirable. The sole limitation on the molecular weight and the chemical structure of the conjugated moiety is that it does not result in a Lipid-conjugate or Phospholipid-conjugate devoid of the desired biological activity, or lead to chemical or physiological instability to the extent that the Lipid-conjugate or Phospholipid-conjugate is rendered useless as a drug in the method of use described herein.

In one embodiment, the compound for use in the present invention is represented by the structure of the general formula (A):

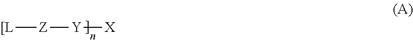

wherein
L is a lipid or a phospholipid;
Z is either nothing, ethanolamine, serine, inositol, choline, phosphate, or glycerol;
Y is either nothing or a spacer group ranging in length from 2 to 30 atoms;
X is a physiologically acceptable monomer, dimer, oligomer, or polymer; and
n is a number from 1 to 1000 or a number from 2 to 1000;
wherein any bond between L, Z, Y and X is either an amide or an esteric bond.

In one embodiment, L of Compound A is phospholipids (PL). In another embodiment, L of Compound A is a lipid.

In one embodiment, L is phosphatidyl, Z is ethanolamine, wherein L and Z are chemically bonded resulting in phosphatidylethanolamine, Y is nothing, and X is carboxymethylcellulose. In another embodiment, L is phosphatidyl, Z is ethanolamine, wherein L and Z are chemically bonded resulting in phosphatidylethanolamine, Y is nothing, and X is a glycosaminoglycan. In one embodiment, the phosphatidylethanolamine moiety is dipalmitoyl phosphatidylethanolamine. In another embodiment, the phosphatidylethanolamine moiety is dimyristoyl phosphatidylethanolamine. In another embodiment, the phosphatidylethanolamine moiety is 1-Acyl-2-Acyl-sn-Glycero-3-Phosphoethanolamine. In another embodiment, the phosphatidylethanolamine moiety is 1,2-Diacyl-sn-Glycero-3-Phosphoethanolamine. In another embodiment, the phosphatidylethanolamine moiety is 1-hexadecanoyl-2-[(Z)-octadec-9-enoyl]-sn-glycero-3-phospho}ethanolamine. In another embodiment, the phosphatidylethanolamine moiety is 1,2-distearoylphosphatidylethanolamine. In another embodiment, the phosphatidylethanolamine moiety is 1,2-distearoylphosphatidylethanolamine zwitterions. In another embodiment, the phosphatidylethanolamine moiety is 1,2-distearoylphosphatidylethanolaminium. In another embodiment, the phosphatidylethanolamine moiety is phosphatidyldi-N-methylethanolamines. In another embodiment, the phosphatidylethanolamine moiety is phosphatidyl-N-methylethanolamines.

In another embodiment, the phosphatidylethanolamine moiety is a transesterified phosphatidylethanolamine. In another embodiment, the phosphatidylethanolamine moiety is dipalmitoyl phosphatidylethanolamine. In another embodiment, the phosphatidylethanolamine moiety is palmitoyl oleoyl phosphatidylethanolamine. In another embodiment, the phosphatidylethanolamine moiety is dioleoyl phosphatidylethanolamine. In another embodiment, the phosphatidylethanolamine moiety is a PE conjugated to a moiety selected from the group comprising of dicarboxylic acids, polyethylene glycols, polyalkyl ethers and gangliosides.

In another embodiment, the phosphatidylethanolamine moiety is a synthetic analogs of phosphatidylethanolamine. In another embodiment, the phosphatidylethanolamine moiety is isolated from natural sources. In another embodiment, the phosphatidylethanolamine moiety is synthesized according to established chemical procedures, or enzymatically synthesized using the corresponding phosphatidyl choline compound in the presence of ethanolamine and phospholipase D.

In another embodiment, the compound for use in the present invention is represented by the structure of the general formula (I):

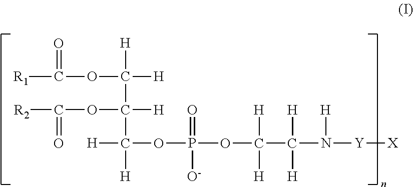

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms; and
X is either a physiologically acceptable monomer, dimer, oligomer or a physiologically acceptable polymer; and
n is a number from 1 to 1,000 or 2 to 1000;
wherein if Y is nothing the phosphatidylethanolamine is directly linked to X via an amide bond and if Y is a spacer, the spacer is directly linked to X via an amide or an esteric bond and to the phosphatidylethanolamine via an amide bond.

In another embodiment, the compound for use in the present invention is represented by the structure of the general formula (I), wherein X is glycosaminoglycan (GAG). In another embodiment the compound for use is represented by the structure of formula I, wherein X is glycosaminoglycan (GAG) and n is a number from 1 to 70. In another embodiment, the molecular weight of said GAG is between 5 to 20 kD

Examples of phosphatidylethanolamine (PE) moieties are analogues of the phospholipid in which the chain length of the two fatty acid groups attached to the glycerol backbone of the phospholipid varies from 2-30 carbon atoms length, and in which these fatty acids chains contain saturated and/or unsaturated carbon atoms. In lieu of fatty acid chains, alkyl chains attached directly or via an ether linkage to the glycerol backbone of the phospholipid are included as analogues of PE. In one embodiment, the PE moiety is dipalmitoyl-phosphatidyl-ethanolamine. In another embodiment, the PE moiety is dimyristoyl-phosphatidyl-ethanolamine.

Phosphatidyl-ethanolamine and its analogues may be from various sources, including natural, synthetic, and semisynthetic derivatives and their isomers.

Phospholipids which can be employed in lieu of the PE moiety are N-methyl-PE derivatives and their analogues, linked through the amino group of the N-methyl-PE by a covalent bond; N,N-dimethyl-PE derivatives and their analogues linked through the amino group of the N,N-dimethyl-PE by a covalent bond.

For PE-conjugates and PS-conjugates, the phospholipid is linked to the conjugated monomer or polymer moiety through the nitrogen atom of the phospholipid polar head group, either directly or via a spacer group. For PC, PI, and PG conjugates, the phospholipid is linked to the conjugated monomer or polymer moiety through either the nitrogen or one of the oxygen atoms of the polar head group, either directly or via a spacer group.

In another embodiment, the compound for use in the present invention is represented by the structure of the general formula (II):

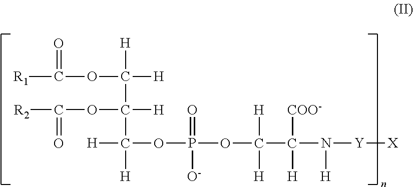

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein if Y is nothing, the phosphatidylserine is directly linked to X via an amide bond and if Y is a spacer, the spacer is directly linked to X via an amide or an esteric bond and to the phosphatidylserine via an amide bond.

In another embodiment, the compound for use in the present invention is represented by the structure of the general formula (II), wherein X is glycosaminoglycan (GAG) and n is a number from 1 to 70. In another embodiment, the molecular weight of said GAG is between 5 to 20 kD.

In one embodiment, the phosphatidylserine may be bonded to Y, or to X if Y is nothing, via the COO⁻ moiety of the phosphatidylserine.

In another embodiment, the compound for use in the present invention is represented by the structure of the general formula (III):

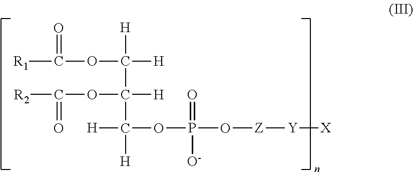

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the phosphatidyl, Z, Y and X is either an amide or an esteric bond.

In another embodiment, the compound for use in the present invention is represented by the structure of the general formula (III), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (IV):

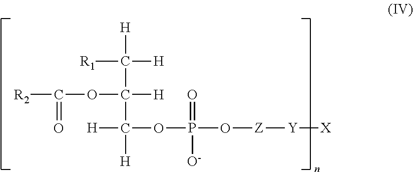

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, ethanolamine, serine or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the phospholipid, Z, Y and X is either an amide or an esteric bond.

In another embodiment, the compound for use in the present invention is represented by the structure of the general formula (IV), wherein X is glycosaminoglycan (GAG) and n is a number from 1 to 70. In another embodiment, the molecular weight of said GAG is between 5 to 20 kD

In another embodiment, the compound for use in the present invention is represented by the structure of the general formula (V):

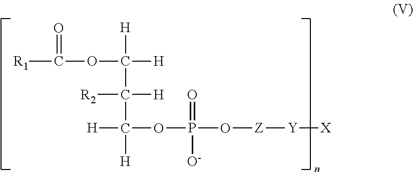

wherein
to R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, ethanolamine, serine or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the phospholipid, Z, Y and X is either an amide or an esteric bond.

In another embodiment, the compound for use in the present invention is represented by the structure of the general formula (V), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (VI):

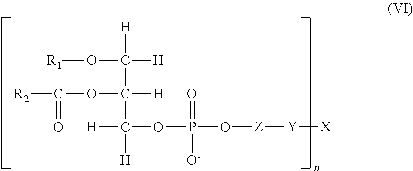

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, ethanolamine, serine or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the phospholipid, Z, Y and X is either an amide or an esteric bond.

In another embodiment, the compound for use in the present invention is represented by the structure of the general formula (VI), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (VII):

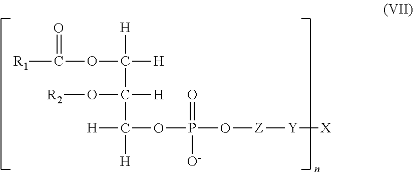

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, ethanolamine, serine, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the phospholipid, Z, Y and X is either an amide or an esteric bond.

In another embodiment, the compound for use in the present invention is represented by the structure of the general formula (VII), wherein X is glycosaminoglycan (GAG) and n is a number from 1 to 70. In another embodiment, the molecular weight of said GAG is between 5 to 20 kD.

In one embodiment of the invention, the conjugate comprises phosphatidylcholine (PC), phosphatidylinositol (PI), phosphatidic acid (PA), wherein Z is nothing, and phosphatidylglycerol (PG) as defined as compounds of the general formula (III).

In another embodiment, the compound for use in the present invention is represented by the structure of the general formula (VIII):

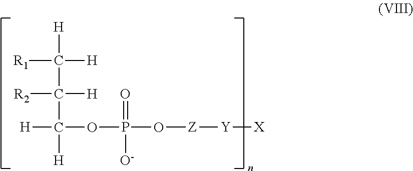

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the phospholipid, Z, Y and X is either an amide or an esteric bond.

In another embodiment, the compound for use in the present invention is represented by the structure of the general formula (VIII), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (IX):

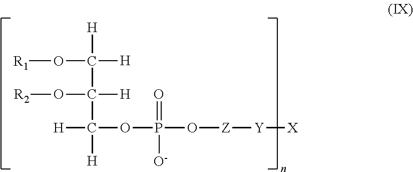

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the phospholipid, Z, Y and X is either an amide or an esteric bond.

In another embodiment, the compound for use in the present invention is represented by the structure of the general formula (IX), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (IXa):

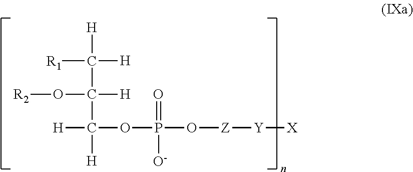

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the phospholipid, Z, Y and X is either an amide or an esteric bond.

In another embodiment, the compound for use in the present invention is represented by the structure of the general formula (IXa), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (IXb):

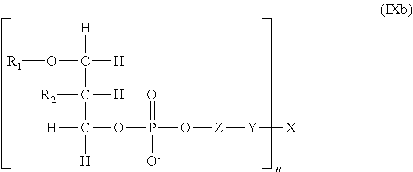

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the phospholipid, Z, Y and X is either an amide or an esteric bond.

In another embodiment, the compound for use in the present invention is represented by the structure of the general formula (IXb), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (X):

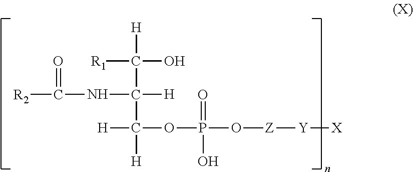

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the ceramide phosphoryl, Z, Y and X is either an amide or an esteric bond.

In another embodiment, the compound for use in the present invention is represented by the structure of the general formula (X), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (Xa):

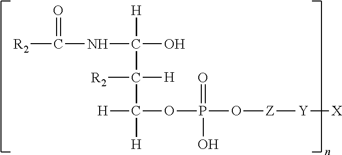

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000 or a number from 2 to 1000;
wherein any bond between the ceramide phosphoryl, Z, Y and X is either an amide or an esteric bond.

In another embodiment, the compound for use in the present invention is represented by the structure of the general formula (Xa), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XI):

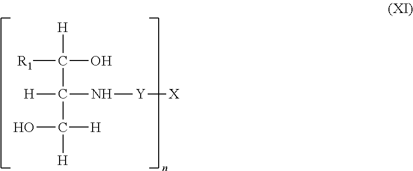

wherein
is R₁ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein if Y is nothing the sphingosyl is directly linked to X via an amide bond and if Y is a spacer, the spacer is directly linked to X and to the sphingosyl via an amide bond and to X via an amide or an esteric bond.

In another embodiment, the compound for use in the present invention is represented by the structure of the general formula (XI), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XII):

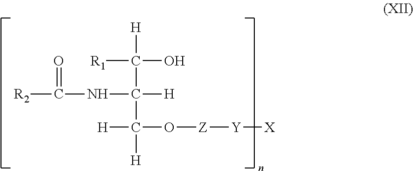

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, phosphate, inositol, choline, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 001000;
wherein any bond between the ceramide, Z, Y and X is either an amide or an esteric bond.

In another embodiment, the compound for use in the present invention is represented by the structure of the general formula (XII), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XIIa):

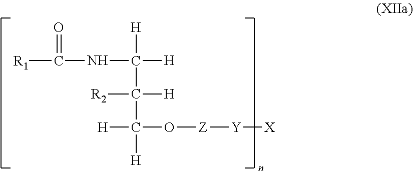

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms:
Z is either nothing, ethanolamine, serine, inositol, phosphate, choline, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 001000;
wherein any bond between the ceramide, Z, Y and X is either an amide or an esteric bond.

In another embodiment, the compound for use in the present invention is represented by the structure of the general formula (XIIa), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XIII):

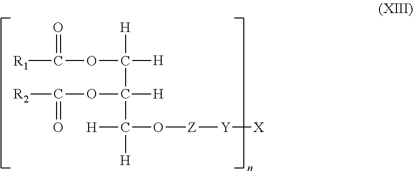

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, ethanolamine, serine, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein any bond between the diglyceryl, Z, Y and X is either an amide or an esteric bond.

In another embodiment, the compound for use in the present invention is represented by the structure of the general formula (XIII), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XIV):

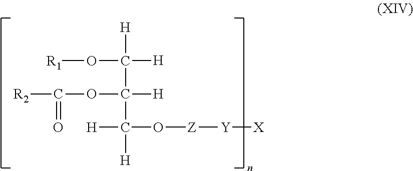

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, ethanolamine, serine, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein any bond between the glycerolipid, Z, Y and X is either an amide or an esteric bond.

In another embodiment, the compound for use in the present invention is represented by the structure of the general formula (XIV), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XV):

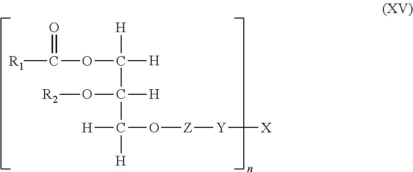

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, ethanolamine, serine, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein any bond between the glycerolipid, Z, Y and X is either an amide or an esteric bond.

In another embodiment, the compound for use in the present invention is represented by the structure of the general formula (XV), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XVI):

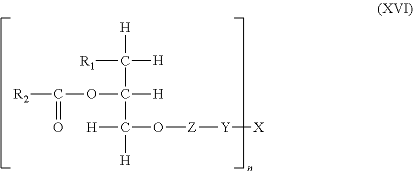

wherein
R₁ is either hydrogen or a linear, saturated, mono-unsaturated, or poly-unsaturated, alkyl chain ranging in length from 2 to 30 carbon atoms:
R₂ is a linear, saturated, mono-unsaturated, or poly-unsaturated, alkyl chain ranging in length from 2 to 30 carbon atoms;
Z is either nothing, choline, ethanolamine, serine, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein any bond between the lipid, Z, Y and X is either an amide or an esteric bond.

In another embodiment, the compound for use in the present invention is represented by the structure of the general formula (XVI), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XVII):

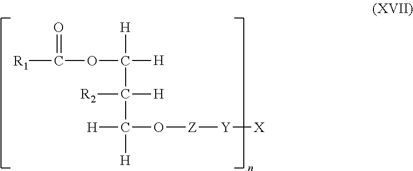

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, ethanolamine, serine, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein any bond between the lipid, Z, Y and X is either an amide or an esteric bond.

In another embodiment, the compound for use in the present invention is represented by the structure of the general formula (XVII), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XVIII):

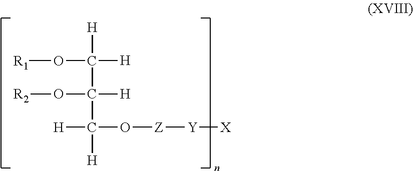

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms:
Z is either nothing, choline, ethanolamine, serine, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein any bond between the lipid, Z, Y and X is either an amide or an esteric bond.

In another embodiment, the compound for use in the present invention is represented by the structure of the general formula (XVIII), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XIX):

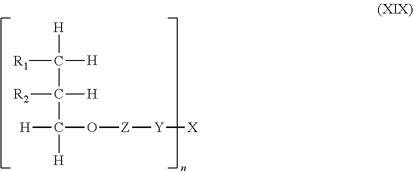

wherein
R₁ is either hydrogen or a linear, saturated, mono-unsaturated, or poly-unsaturated, alkyl chain ranging in length from 2 to 30 carbon atoms:
R₂ is either hydrogen or a linear, saturated, mono-unsaturated, or poly-unsaturated, alkyl chain ranging in length from 2 to 30 carbon atoms;
Z is either nothing, choline, ethanolamine, seine, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein any bond between the lipid, Z, Y and X is either an amide or an esteric bond.

In another embodiment, the compound for use in the present invention is represented by the structure of the general formula (XIX), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XX):

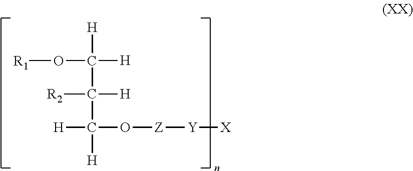

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, ethanoleamine, serine, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein any bond between the lipid, Z, Y and X is either an amide or an esteric bond.

In another embodiment, the compound for use in the present invention is represented by the structure of the general formula (XX), wherein X is glycosaminoglycan (GAG) and n is a number from 1 to 70. In another embodiment, the molecular weight of said GAG is between 5 to 20 kD.

In another embodiment, the compound for use in the present invention is represented by the structure of the general formula (XXI):

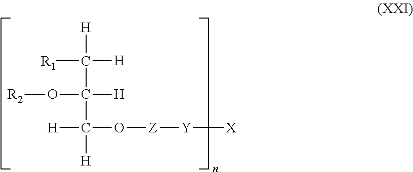

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, ethanolamine, serine, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000 or a number from 2 to 1000;
wherein any bond between the lipid, Z, Y and X is either an amide or an esteric bond.

In another embodiment, the compound for use in the present invention is represented by the structure of the general formula (XXI), wherein X is glycosaminoglycan (GAG) and n is a number from 1 to 70. In another embodiment, the molecular weight of said GAG is between 5 to 20 kD.

For any or all of the compounds represented by the structures of the general formulae (A), (I), (II), (III), (IV), (V), (VI), (VII), (VIII), (IX), (IXa), (IXb), (X), (Xa), (XI), (XII), (XIIa), (XIII), (XIV), (XV), (XVI), (XVII), (XVIII), (XIX), (XX), (XXI), and (XXII) hereinabove: In one embodiment, X is a glycosaminoglycan. According to this aspect and in one embodiment, the glycosaminoglycan may be, inter alia, hyaluronic acid, heparin, heparan sulfate, chondroitin sulfate, keratin, keratan sulfate, dermatan sulfate or a derivative thereof. In one embodiment, the chondroitin sulfate may be, inter alia, chondroitin-6-sulfate, chondroitin-4-sulfate or a derivative thereof. In another embodiment, X is not a glycosaminoglycan. In another embodiment, X is a polysaccharide, which in one embodiment is a hetero-polysaccharide, and in another embodiment, is a homo-polysaccharide. In another embodiment, X is a polypyranose.

In another embodiment, the glycosaminoglycan is a polymer of disaccharide units. In another embodiment, the number of the disaccharide units in the polymer is m. In another embodiment, m is a number from 2-10,000. In another embodiment, m is a number from 2-500. In another embodiment, m is a number from 2-1000. In another embodiment, m is a number from 50-500. In another embodiment, m is a number from 2-2000. In another embodiment, m is a number from 500-2000. In another embodiment, m is a number from 1000-2000. In another embodiment, m is a number from 2000-5000. In another embodiment, m is a number from 3000-7000. In another embodiment, m is a number from 5000-10,000. In another embodiment, a disaccharide unit of a glycosaminoglycan may be bound to one lipid or phospholipid moiety. In another embodiment, each disaccharide unit of the glycosaminoglycan may be bound to zero or one lipid or phospholipid moieties. In another embodiment, the lipid or phospholipid moieties are bound to the —COOH group of the disaccharide unit. In another embodiment, the bond between the lipid or phospholipid moiety and the disaccharide unit is an amide bond.

In one embodiment of the invention, V is nothing. Non-limiting examples of suitable divalent groups forming the optional bridging group (which in one embodiment, is referred to as a spacer) Y, according to embodiments of the invention, are straight or branched chain alkylene, e.g., of 2 or more, preferably 4 to 30 carbon atoms, —CO-alkylene-CO, —NH-alkylene-NH—, —CO-alkylene-NH—, —NH-alkylene-NH, CO-alkylene-NH—, an amino acid, cycloalkylene, wherein alkylene in each instance, is straight or branched chain and contains 2 or more, preferably 2 to 30 atoms in the chain, —(—O—CH(CH₃)CH₂—)ₓ— wherein x is an integer of 1 or more.

In one embodiment of the invention, the sugar rings of the glycosaminoglycan are intact. In another embodiment, intact refers to closed. In another embodiment, intact refers to natural. In another embodiment, intact refers to unbroken.

In one embodiment of the invention, the structure of the lipid or phospholipid in any compound according to the invention is intact. In another embodiment, the natural structure of the lipid or phospholipids in any compound according to the invention is maintained.

In some embodiments, the compounds (A), (III), (IV), (V), (VI), (VII), (VIII), (IX), (IXa), (IXb), (X), (Xa), (XI), (XII), (XIIa), (XIII), (XIV), (XV), (XVI), (XVII), (XVIII), (XIX), (XX) and (XXI) as presented hereinabove comprises a Z group. In one embodiment, Z is a nothing. In another embodiment Z is inositol. In another embodiment, Z is choline. In another embodiment, Z is glycerol.

In some embodiments, the compounds (XII), (XIIa), (XIII), (XIV), (XV), (XVI), (XVII), (XVIII), (XIX), (XX) and (XXI) as presented hereinabove comprises a Z group. In one embodiment, the Z is a phosphate. In another embodiment, the phosphate is phoso-ethanolamine —P(OH)(═O)—O—CH₂CH₂—NH—. In another embodiment, the phosphate is phospho-serine-P(OH)(═O)—O—CH₂CH(COOH)—NH—.

In one embodiment, compounds (A), (III), (IV), (V), (VI), (VII), (VIII), (IX), (IXa), (IXb), (X), (Xa), (XI), (XII), (XIIa), (XIII), (XIV), (XV), (XVI), (XVII), (XVIII), (XIX), (XX) and (XXI) for use in the methods of the invention comprise one of the following as the conjugated moiety X. In another embodiment X is acetate, butyrate, glutarate, succinate, dodecanoate, didodecanoate, maltose, lactobionic acid, dextran, alginate, aspirin, cholate, cholesterylhemisuccinate, carboxymethyl-cellulose, heparin, hyaluronic acid, chondroitin sulfate, polygeline (haemaccel), polyethyleneglycol, polycarboxylated polyethylene glycol, a glycosaminoglycan, a polysaccharide, a hetero-polysaccharide, a homo-polysaccharide, or a polypyranose.

The polymers used as starting material to prepare the lipids or PL-conjugates may vary in molecular weight from 1 to 2,000 kDa.

In another embodiment, the phospholipid (PL)-conjugate compound of this invention is a phosphatidylethanolamine, a phosphatidylserine, a phosphatidylcholine, a phosphatidylinositol, a phosphatidic acid or a phosphatidylglycerol. In another embodiment, PL comprises the residue of palmitic acid, myristic acid, myristoleic acid, palmitoleic acid, oleic acid, linoleic acid, linolenic acid, arachidonic acid, eicosapentaenoic acid, erucic acid or docosahexaenoic acid. In another embodiment, PL is dimyristoyl phosphatidylethanolamine. In another embodiment, PL is dipalmitoyl phosphatidylethanolamine. Phosphatidylserine (PS) and its analogues, such as palmitoyl-stearoyl-PS, natural PS from various sources, semisynthetic PSs, synthetic, natural and artificial PSs and their isomers.

In one embodiment, the compounds of this invention comprise lipid conjugates. In another embodiment, the lipid is lysophospholipids, sphingomyelins, lysosphingomyelins, ceramide, and sphingosine.

For PE-conjugates and PS-conjugates, the phospholipid is linked to the conjugated monomer or polymer moiety through the nitrogen atom of the phospholipid polar head group, either directly or via a spacer group. For PC, PI, and PG conjugates, the phospholipid is linked to the conjugated monomer or polymer moiety through either the nitrogen or one of the oxygen atoms of the polar head group, either directly or via a spacer group. The PS can bind also via the COOH group.

In one embodiment, the lipid and PL are conjugated to glycosaminoglycan (GAG). In another embodiment, the GAG is hyaluronic acid, heparin, heparan sulfate, chondroitin, chondroitin sulfate, dermatan sulfate or keratan sulfate. In another embodiment, GAG is hyaluronic acid. In another embodiment, GAG is heparin. In another embodiment, GAG is chondroitin. In another embodiment, GAG is chondroitin sulfate. In another embodiment, GAG is dermatan sulfate, in another embodiment, GAG is keratan sulfate.

In another embodiment, chondroitin sulfate is chondroitin-6-sulfate, chondroitin-4-sulfate or a derivative thereof. In another embodiment, dermatan sulfate is dermatan-6-sulfate, dermatan-4-sulfate or a derivative thereof.

In one embodiment, the compounds for use in the present invention are biodegradable.

In one embodiment, the compound according to the invention is phosphatidylethanolamine bound to aspirin. In one embodiment, the compound according to the invention is phosphatidylethanolamine bound to glutarate.

In some embodiments, the compounds for use are as listed in Table 1 below.

TABLE 1

| Phospholipid | Spacer | Polymer (m.w.) | Compound |
|---|---|---|---|
| PE | None | Hyaluronic acid | XXII |
| | | (2-2000 kDa) | |
| Dimyristoyl-PE | None | Hyaluronic acid | XXIII |
| PE | None | Heparin | XXIV |
| | | (0.5-110 kDa) | |
| PE | None | Chondroitin sulfate A | XXV |
| PE | None | Carboxymethylcellulose | XXVI |
| | | (20-500 kDa) | |
| PE | Dicarboxylic acid + | Polygeline (haemaccel) | XXVII |
| | Diamine | (4-40 kDa) | |
| PE | None | Hydroxyethylstarch | XXVIII |
| PE | Dicarboxylic acid + | Dextran | XXIX |
| | Diamine | (1-2,000 kDa) | |
| PE | None | Aspirin | XXX |
| PE | Carboxyl amino | Hyaluronic acid | XXXI |
| | group | (2-2000 kDa) | |
| PE | Dicarboxyl group | Hyaluronic acid | XXXII |
| | | (2-2000 kDa) | |
| PE | Dipalmitoic acid | Hyaluronic acid | XXXIII |
| | | (2-2000 kDa) | |
| PE | Carboxyl amino | Heparin | XXXIV |
| | group | (0.5-110 kDa) | |
| PE | Dicarboxyl group | Heparin | XXXV |
| | | (0.5-110 kDa) | |
| PE | Carboxyl amino | Chondroitin sulfate A | XXXVI |
| | group | | |
| PE | Dicarboxyl group | Chondroitin sulfate A | XXXVII |
| PE | Carboxyl amino | Carboxymethylcellulose | XXXVIII |
| | group | (20-500 kDa) | |
| PE | Dicarboxyl group | Carboxymethylcellulose | XXXIX |
| | | (20-500 kDa) | |
| PE | None | Polygeline (haemaccel) | XL |
| | | (4-40 kDa) | |
| PE | Carboxyl amino | Polygeline (haemaccel) | XLI |
| | group | (4-40 kDa) | |
| PE | Dicarboxyl group | Polygeline (haemaccel) | XLII |
| | | (4-40 kDa) | |
| PE | Carboxyl amino | Hydroxyethylstarch | XLIII |
| | group | | |
| PE | Dicarboxyl group | Hydroxyethylstarch | XLIV |
| PE | None | Dextran | XLV |
| | | (1-2,000 kDa) | |
| PE | Carboxyl amino | Dextran | XLVI |
| | group | (1-2,000 kDa) | |
| PE | Dicarboxyl group | Dextran | XLVII |
| | | (1-2,000 kDa) | |
| PE | Carboxyl amino | Aspirin | XLVIII |
| | group | | |
| PE | Dicarboxyl group | Aspirin | XLIX |
| PE | None | Albumin | L |
| PE | None | Alginate | LI |
| | | (2-2000 kDa) | |
| PE | None | Polyaminoacid | LII |
| PE | None | Polyethylene glycol | LIII |
| PE | None | Lactobionic acid | LIV |
| PE | None | Acetylsalicylate | LV |
| PE | None | Cholesteryl- | LVI |
| | | hemmisuccinate | |
| PE | None | Maltose | LVII |
| PE | None | Cholic acid | LVIII |
| PE | None | Chondroitin sulfates | LIX |
| PE | None | Polycarboxylated | LX |
| | | polyethylene glycol | |
| Dipalmitoyl-PE | None | Hyaluronic acid | LXI |
| Dipalmitoyl-PE | None | Heparin | LXII |
| Dipalmitoyl-PE | None | Chondroitin sulfate A | LXIII |
| Dipalmitoyl-PE | None | Carboxymethylcellulose | LXIV |
| Dipalmitoyl-PE | None | Polygeline (haemaccel) | LXV |
| Dipalmitoyl-PE | None | Hydroxyethylstarch | LXVI |
| Dipalmitoyl-PE | None | Dextran | LXVII |
| Dipalmitoyl-PE | None | Aspirin | LXVIII |
| Dimyristoyl-PE | None | Heparin | LXVIX |
| Dimyristoyl-PE | None | Chondroitin sulfate A | LXX |
| Dimyristoyl-PE | None | Carboxymethylcellulose | LXXI |
| Dimyristoyl-PE | None | Polygeline (haemaccel) | LXXII |
| Dimyristoyl-PE | None | Hydroxyethylstarch | LXXIII |
| Dimyristoyl-PE | None | Dextran | LXXIV |
| Dimyristoyl-PE | None | Aspirin | LXXV |
| PS | None | Hyaluronic acid | LXXVI |
| PS | None | Heparin | LXXVII |
| PS | None | Polygeline (haemaccel) | LXXVIII |
| PC | None | Hyaluronic acid | LXXIX |
| PC | None | Heparin | LXXX |
| PC | None | Polygeline (haemaccel) | LXXXI |
| PI | None | Hyaluronic acid | LXXXII |
| PI | None | Heparin | LXXXIII |
| PI | None | Polygeline (haemaccel) | LXXXIV |
| PG | None | Hyaluronic acid | LXXXV |
| PG | None | Heparin | LXXXVI |
| PG | None | Polygeline (haemaccel) | LXXXVII |
| PE | None | Glutaryl | LXXXVIII |

In one embodiment of the invention, the compounds for use in the present invention are any one or more of Compounds I-LXXXVIII. In another embodiment, the compounds for use in the present invention are Compound XXII, Compound XXIII, Compound XXIV, Compound XXV, Compound XXVI, Compound XXVII, Compound XXVIII, Compound XXIX, Compound XXX, or pharmaceutically acceptable salts thereof, in combination with a physiologically acceptable carrier or solvent. According to embodiments of the invention, these polymers, when chosen as the conjugated moiety, may vary in molecular weights from 200 to 2,000,000 Daltons. In one embodiment of the invention, the molecular weight of the polymer as referred to herein is from 200 to 1000 Daltons. In another embodiment, the molecular weight of the polymer as referred to herein is from 200 to 1000 Daltons. In another embodiment, the molecular weight of the polymer as referred to herein is from 1000 to 5000 Daltons. In another embodiment, the molecular weight of the polymer as referred to herein is from 5000 to 10,000 Daltons. In another embodiment, the molecular weight of the polymer as referred to herein is from 10,000 to 20,000 Daltons. In another embodiment, the molecular weight of the polymer as referred to herein is from 10,000 to 50,000 Daltons. In another embodiment, the molecular weight of the polymer as referred to herein is from 20,000 to 70,000 Daltons. In another embodiment, the molecular weight of the polymer as referred to herein is from 50,000 to 100,000 Daltons. In another embodiment, the molecular weight of the polymer as referred to herein is from 100,000 to 200,000 Daltons. In another embodiment, the molecular weight of the polymer as referred to herein is from 200,000 to 500,000 Daltons. In another embodiment, the molecular weight of the polymer as referred to herein is from 200,000 to 1,000,000 Daltons. In another embodiment, the molecular weight of the polymer as referred to herein is from 500,000 to 1,000,000 Daltons. In another embodiment, the molecular weight of the polymer as referred to herein is from 1,000,000 to 2,000,000 Daltons. Various molecular weight species have been shown to have the desired biological efficacy.

Examples of suitable divalent groups forming the optional bridging group Y are straight- or branched-chain alkylene, e.g., of 2 or more, preferably 4 to 18 carbon atoms, —CO-alkylene-CO, —NH-alkylene-NH—, —CO-alkylene-NH—, cycloalkylene, wherein alkylene in each instance, is straight or branched chain and contains 2 or more, preferably 2 to 18 carbon atoms in the chain, —(—O—CH(CH₃)CH₂—)ₓ— wherein x is an integer of 1 or more.

In another embodiment, in addition to the traditional phospholipid structure, related derivatives for use in this invention are phospholipids modified at the C1 or C2 position to contain an ether or alkyl bond instead of an ester bond. In one embodiment of the invention, the alkyl phospholipid derivatives and ether phospholipid derivatives are exemplified herein. In one embodiment, these derivatives are exemplified hereinabove by the general formulae (VIII) and (IX).

In one embodiment of the invention, X is covalently conjugated to a lipid. In another embodiment, X is covalently conjugated to a lipid via an amide bond. In another embodiment, X is covalently conjugated to a lipid via an esteric bond. In another embodiment, the lipid is phosphatidylethanolamine.

In one embodiment, cell surface GAGs play a key role in protecting cells from diverse damaging agents and processes, such as reactive oxygen species and free radicals, endotoxins, cytokines, invasion promoting enzymes, and agents that induce and/or facilitate degradation of extracellular matrix and basal membrane, cell invasiveness, white cell extravasation and infiltration, chemotaxis, and others. In addition, cell surface GAGs protect cells from bacterial, viral and parasitic infection, and their stripping exposes the cell to interaction and subsequent internalization of the microorganism. Enrichment of cell surface GAGs would thus assist in protection of the cell from injurious processes. Thus, in one embodiment of the invention, PLA2 inhibitors are conjugated to GAGs or GAG-mimicking molecules. In another embodiment, these Lipid-conjugates provide wide-range protection from diverse injurious processes, and ameliorate diseases that require cell protection from injurious biochemical mediators.

In another embodiment, a GAG-mimicking molecule may be, inter alia, a negatively charged molecule. In another embodiment, a GAG-mimicking molecule may be, inter alia, a salicylate derivative. In another embodiment, a GAG-mimicking molecule may be, inter alia, a dicarboxylic acid.

In another embodiment, a composition as described herein further comprises zinc oxide, Vitamins A, D, E, and K, an antibacterial agent, or any combination thereof. In another embodiment, an antibacterial agent as described herein is a bismuth-containing compound, sulfonamides, nitrofurans, metronidazole, nimorazole, tinidazole, benzoic acid, aminoglycosides, macrolides, penicillins, polypeptides, tetracyclines, cephalosporins, chloramphenicol, clindamycin and mixtures thereof. In more preferred embodiments, the antibacterial agents are selected from the group consisting of bismuth aluminate, bismuth subcitrate, bismuth subgalate, bismuth subsalicylate, sulfonamides, nitrofurazone, nitrofurantoin, furazolidone, metronidazole, tinidazole, nimorazole, benzoic acid, hentamycin, neomycin, kynamycin, streptomycin, erythromycin, clindamycin, rifampin, rifamycin, penicillin G, penicillin V, ampicillin, amoxicillin, bacitracin, polymyxin, tetracycline, chlortetracycline, oxytetracycline, doxycycline, cephalexin, cephalothin, clindamycin, chloramphenical and mixtures thereof.

In another embodiment, the antibacterial agent is selected from a wide range of therapeutic agents and mixtures of therapeutic agents which may be administered in sustained release or prolonged action form. Nonlimiting illustrative specific examples of antibacterial agents include bismuth containing compounds, sulfonamides; nitrofurans, metronidazole, tinidazole, nimorazole, benzoic acid; aminoglycosides, macrolides, penicillins, polypeptides, tetracyclines, cephalosporins, chloramphenicol, and clindamycin. Preferably, the antibacterial agent is selected from the group consisting of bismuth containing compounds, such as, without limitation, bismuth aluminate, bismuth subcitrate, bismuth subgalate, bismuth subsalicylate, and mixtures thereof; the sulfonamides; the nitrofurans, such as nitrofurazone, nitrofurantoin, and furozolidone; and miscellaneous antibacterials such as metrotidazole, tinidazole, nimorazole, and benzoic acid; and antibiotics, including the aminoglycosides, such as gentamycin, neomycin, kanamycin, and streptomycin; the macrolides, such as erythromycin, clindamycin, and rifamycin; the penicillins, such as penicillin G, penicillin V, Ampicillin and amoxicillin; the polypeptides, such as bactracin and polymyxin; the tetracyclines, such as chlorotetracycline, oxytetracycline, and doxycycline; the cephalospoins, suck as cephalexin and cephalothin; and miscellaneous antibiotics, such as chloramphenicol, and clindamycin. More preferably, the antibacterial agent is selected from the group consisting of bismuth aluminate, nitrofurantoin, furozolidone, metronidazole, tinidazole, nimorazole, benzoic acid, gentamycin, neomycin, kanamycin, streptomycin, erythromycin, clindamycin, rifamycin, penicillin G, penicillin V, Ampicillin amoxicillin, bacitracin, polymyxin, tetracycline, chlorotetracycline, oxytetracycline, doxycycline, cephalexin, cephalothin, chloramphenicol, and clidamycin.

In another embodiment, the antifungal agent is astemizole, clotrimazole, omeprazole, econazole, oxiconazole, sulconazole, fluconazole, ketoconazole, itraconazole, torbinafine, and mixtures thereof. In another embodiment, a composition as described herein comprises a calcium channel blocker.

In another embodiment, the invention provides a pharmaceutical composition comprising a lipid or phospholipid moiety bonded to a physiologically acceptable monomer, dimer, oligomer, or polymer; and a pharmaceutically acceptable carrier or excipient. In another embodiment, the invention provides a pharmaceutical composition comprising a conjugate as described for treating a subject afflicted with a tumor. In another embodiment, the invention provides a pharmaceutical composition comprising a conjugate as described for treating a subject in risk of developing a tumor. In another embodiment, the invention provides a pharmaceutical composition comprising a conjugate as described for inhibiting MMP production and/or MMP activity in a cell. In another embodiment, the invention provides a pharmaceutical composition comprising a conjugate as described for treating a subject afflicted with atherosclerosis. In another embodiment, a pharmaceutical composition comprising a conjugate as described is effective in inhibiting blood vessels formation. In another embodiment, a pharmaceutical composition comprising a conjugate as described is effective in inhibiting endothelial cell migration. In another embodiment, a pharmaceutical composition comprising a conjugate as described counteracts the effect of MMP.

In another embodiment, the invention provides a pharmaceutical composition comprising a combination of active pharmaceutical ingredients comprising a lipid or phospholipid moiety bonded to a physiologically acceptable monomer, dimer, oligomer, or polymer; and an anti-cancer agent. In another embodiment, the invention provides a pharmaceutical composition comprising a combination of active pharmaceutical ingredients comprising a lipid or phospholipid moiety bonded to a physiologically acceptable monomer, dimer, oligomer, or polymer; and a an anti-tumor agent. In another embodiment, the invention provides a pharmaceutical composition comprising a combination of active pharmaceutical ingredients comprising a lipid or phospholipid moiety bonded to a physiologically acceptable monomer, dimer, oligomer, or polymer; and a cardiovascular therapeutic agent.

In another embodiment, the invention provides a pharmaceutical composition for treating a subject afflicted with cancer characterized by tumors or afflicted with atherosclerosis, including any one of the compounds for use in the present invention or any combination thereof; and a pharmaceutically acceptable carrier or excipient. In another embodiment, the compounds for use in the present invention include, inter alia, the compounds represented by the structures of the general formulae as described hereinbelow: (A), (I), (II), (III), (IV), (V), (VI), (VII), (VIII), (IX), (IXa), (IXb), (X), (Xa) (XI), (XII), (XIIa), (XIII), (XIV), (XV), (XVI), (XVII), (XVIII), (XIX), (XX), (XXI), (XXII), or any combination thereof.

### Preparation of Compounds for Use in the Present Invention

In one embodiment, the preparation of high molecular weight Lipid-conjugates for use in the methods of the present invention is as described in U.S. Pat. No. 5,064,817, which is incorporated fully herein by reference. In one embodiment, these synthetic methods are applicable to the preparation of Lipid-conjugates as well, i.e. Lipid-conjugates comprising monomers and dimers as the conjugated moiety, with appropriate modifications in the procedure as would be readily evident to one skilled in the art. The preparation of some Lipid-conjugates may be conducted using methods well known in the art or as described in U.S. Provisional Patent Application 60/704,874, which is incorporated herein by reference in its entirety.

### Dosages and Routes of Administration

The methods of this invention can be adapted to the use of the therapeutic compositions comprising Lipid-conjugates or Phospholipid-conjugates in admixture with conventional excipients, i.e. pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins, steroids, anti-inflammatory compounds, etc., as will be understood by one skilled in the art.

In one embodiment, the route of administration may be parenteral, enteral, or a combination thereof. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation, nasal aspiration (spray), sublingual, oral, aerosol or suppository or a combination thereof. In one embodiment, the dosage regimen will be determined by skilled clinicians, based on factors such as exact nature of the condition being treated, the severity of the condition, the age and general physical condition of the patient, etc.

In another embodiment, the compositions include those suitable for oral, rectal, intravaginal, topical, nasal, ophthalmic or parenteral administration, all of which may be used as routes of administration using the materials of the present invention. Other suitable routes of administration include direct injection onto an arterial surface and intraparenchymal injection directly into targeted areas of an organ or a tumor. The term “parenteral” includes subcutaneous injections, intravenous, intramuscular, intrasternal injection or infusion techniques.

The compositions may conveniently be presented in unit dosage form and may be prepared by any of the methods well known in the art of pharmacy. Methods typically include the step of bringing the active ingredients of the invention into association with a carrier which constitutes one or more accessory ingredients.

Compositions of the present invention suitable for oral administration may be presented as discrete units such as capsules, cachets, tablets or lozenges, each containing a predetermined amount of the compounds of the invention in liposomes or as a suspension in an aqueous liquid or non-aqueous liquid such as a syrup, an elixir, or an emulsion.

Compositions suitable for parenteral administration conveniently comprise a sterile aqueous preparation of the molecule of the invention which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Oral agents provide the advantages of easy administration and chronic systemic treatment. However, local delivery of MMP inhibitors via catheters, gene transfer techniques, and endovascular stents or polymers can be utilized in order to control localized disease.

An exemplary pharmaceutical composition is a therapeutically effective amount of a composition as described herein will inhibit MMP as shown in a standard assay, which optionally is included in a pharmaceutically-acceptable and compatible carrier.

The term “pharmaceutically-acceptable and compatible carrier” as used herein, includes one or more compatible solid or liquid filler diluents or encapsulating substances that are suitable for administration to a human or other animal. In the present invention, the term “carrier” thus denotes an organic or inorganic ingredient, natural or synthetic, with which the compounds of the invention are combined to facilitate application. The term “therapeutically-effective amount” is that amount of the present pharmaceutical composition which produces a desired result or exerts a desired influence on the particular condition being treated. In another embodiment, when the composition is being used as prophylactic additional doses will be administered at periodic intervals after the initial administration. Various concentrations may be used in preparing compositions incorporating the same ingredient to provide for variations in the age of the patient to be treated, the severity of the condition, the duration of the treatment and the mode of administration.

The term “compatible”, as used herein, means that the components of the pharmaceutical compositions are capable of being commingled with a small molecule of the present invention, and with each other, in a manner such that does not substantially impair the desired pharmaceutical efficacy.

Doses of the pharmaceutical compositions of the invention will vary depending on the subject and upon the particular route of administration used. Dosages can range from 0.1 to 100,000 μg/kg per day, more preferably 1 to 10,000 μg/kg. By way of an example only, an overall dose range of from about, for example, 1 microgram to about 300 micrograms might be used for human use. This dose can be delivered at periodic intervals based upon the composition. In another embodiment, compounds might be administered daily. Pharmaceutical compositions of the present invention can also be administered to a subject according to a variety of other, well-characterized protocols. For example, using pulsed therapy.

In general, the doses utilized for the above described purposes will vary, but will be in an effective amount to exert the desired anti-disease effect. As used herein, the term “pharmaceutically effective amount” refers to an amount of a compound of formulae I-XXI which will produce the desired alleviation in symptoms or signs of disease in a patient. The doses utilized for any of the above-described purposes will generally be from 1 to about 1000 milligrams per kilogram of body weight (mg/kg), administered one to four times per day, or by continuous IV infusion. When the compositions are dosed topically, they will generally be in a concentration range of from 0.1 to about 10% w/v, administered 1-4 times per day.

Desired time intervals for delivery of multiple doses of a particular composition can be determined by one of ordinary skill in the art employing no more than routine experimentation. The conjugate can be comprised of non-antigenic polymeric substances such as dextran, polyvinyl pyrrolidones, polysaccharides, starches, polyvinyl alcohols, polyacryl amides or other similar substantially non-immunogenic polymers. Polyethylene glycol (PEG) is preferred. Other poly(alkylenes oxides) include monomethoxy-polyethylene glycol polypropylene glycol, block copolymers of polyethylene glycol, and polypropylene glycol and the like. The polymers can also be distally capped with C1-4 alkyls instead of monomethoxy groups. The poly(alkylene oxides) used must be soluble in liquid at room temperature. Thus, they preferably have a molecular weight from about 200 to about 20,000 daltons, more preferably about 2,000 to about 10,000 and still more preferably about 5,000.

In one embodiment, the invention provides for the administration of a salt of a compound as described herein as well. In one embodiment, the salt is a pharmaceutically acceptable salt, which, in turn may refer to non-toxic salts of compounds (which are generally prepared by reacting the free acid with a suitable organic or inorganic base) and include, but are not limited to, the acetate, benzenesulfonate, benzoate, bicarbonate, bisulfate, bitartrate, borate, bromide, calcium, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandlate, mesylate, methylbromide, methylnitrate, methylsulfate, mucate, napsylate, nitrate, oleate, oxalate, pamaote, palmitate, panthothenate, phosphate, diphospate, polygalacturonate, salicylate, stearate, subacetate, succinate, tannate, tartrate, teoclate, tosylate, triethiodide, and valerate salts, as well as mixtures of these salts.

In one embodiment, the use of a single chemical entity with potent anti-oxidant, membrane-stabilizing, anti-proliferative, anti-chemokine, anti-migratory, and anti-inflammatory activity provides the desired protection for a subject afflicted with arthritis, or in another embodiment, the methods of this invention provide for use of a combination of the compounds described. In another embodiment, the compounds for use in the present invention may be provided in a single formulation/composition, or in another embodiment, multiple formulations may be used. In one embodiment, the formulations for use in the present invention may be administered simultaneously, or in another embodiment, at different time intervals, which may vary between minutes, hours, days, weeks or months.

In one embodiment the compositions comprising the compounds for use in the present invention may be administered via different routes, which in one embodiment, may be tailored to provide different compounds at different sites, for example some compounds may be given by inta-joint injection to provide for superior relief in-situ, and in another embodiment, some formulations/compounds/compositions may be provided via various topical formulations, or in another embodiment, systemically, to provide for broader effect.

In one embodiment, the compounds for use in the invention may be used for acute treatment of temporary conditions, or may be administered chronically, as needed. In one embodiment of the invention, the concentrations of the compounds will depend on various factors, including the nature of the condition to be treated, the condition of the patient, the route of administration and the individual tolerability of the compositions.

In one embodiment, the methods of this invention provide for the administration of the compounds throughout the life of the subject, or in another embodiment, episodically, in response to severity or constancy of symptomatic stages, or in another embodiment, at the onset of pain associated with arthritis. In another embodiment, the patients to whom the lipid or PL conjugates should be administered are those that are experiencing symptoms of disease or who are at risk of contracting the disease or experiencing a recurrent episode or exacerbation of the disease, or pathological conditions associated with the same.

As used herein, the term “pharmaceutically acceptable carrier” refers to any formulation which is safe, and provides the appropriate delivery for the desired route of administration of an effective amount of at least one compound of the present invention. As such, all of the above-described formulations of the present invention are hereby referred to as “pharmaceutically acceptable carriers.” This term refers to as well the use of buffered formulations wherein the pH is maintained at a particular desired value, ranging from pH 4.0 to pH 9.0, in accordance with the stability of the compounds and route of administration.

For parenteral application, particularly suitable are injectable, sterile solutions, preferably oily or aqueous solutions, as well as suspensions, emulsions, or implants, including suppositories. Ampoules are convenient unit dosages.

For topical application, particularly for the treatment of skin diseases such as but not limited to contact dermatitis or psoriasis, admixture of the compounds with conventional creams or delayed release patches is acceptable.

For enteral application, particularly suitable are tablets, dragees, liquids, drops, suppositories, or capsules. Syrup, elixir, or the like can be used when a sweetened vehicle is employed. When indicated, suppositories or enema formulations may be the recommended route of administration.

Sustained or directed release compositions can be formulated, e.g., liposomes or those wherein the active compound is protected with differentially degradable coatings, e.g., by microencapsulation, multiple coatings, etc. It is also possible to freeze-dry the new compounds and use the lyophilisates obtained, for example, for the preparation of products for injection.

Articular injection are used for treating an osteoarthritic joint with at least one of the compounds as described herein in a concentration of 1-50 mg/ml in a volume of 1-10 ml/injection.

The compounds as described herein may be administered in different ways, for example, periarticular injection, peritendonous injection, periligamentous injection or intramuscular perfusion. Methods of making such injections are known to one of ordinary skill in the art. Such injections are generally subcutaneous and target the vicinity of a joint, especially near the insertions or origins of muscle tendons and ligaments. Local analgesics may be provided at the site of injection. Such analgesics are known to one of ordinary skill in the art.

Further active substances that can be used in an injectable dosage form are: anti-cancer drugs, small molecules, antibiotics, antiseptics, sodium hyaluronate, a glucocorticoidor any combination thereof. Excipients include but are not limited to: isotonizing agents, such as sodium chloride, mannitol, or sorbitol, water for injection as solvent, sodium monohydrogenphosphate, and sodium dihydrogenphosphate. The solution may additionally contain pH modifiers, such as sodium hydroxide, sodium hydrogenphosphate, hydrochloric acid, or citric acid, surfactants, such as polysorbate 80; sodium edetate as stabilizer (synergistic anti-oxidative agent); propylene glycol or polyethylene glycol as cosolvent; and/or antimicrobial agents, like benzyl alcohol, methyl- and propyl-4-hydroxybenzoate, or cetylpyridinium chloride. In the treatment of larger joints, such as the knee, hip or shoulder, syringes of 10-40 mg/2.0 ml are used.

Suspension formulations additionally contain stabilizers, such as carmellose sodium, hypromellose or gelatine, to avoid or reduce the sedimentation of the suspension as far as possible, and to allow for a fast and reliable re-dispersion of the suspension prior to application. It is essential that the crystals in the suspension formulations maintain their particle size. An uncontrolled growth of crystals bears the risk of poor biocompatibility of the suspension formulation upon intra-articular injection.

The injectable formulations can be also formulated as a dry powder which has to be re-dispersed by addition of the dispersing medium (e.g., water for injection). For suspension formulations, it is essential that they are re-dispersed directly before the application, and that the resulting suspension appears homogenous.

It will be appreciated that the actual preferred amounts of active compound in a specific case will vary according to the specific compound being utilized, the particular compositions formulated, the mode of application, and the particular situs and organism being treated. Dosages for a given host can be determined using conventional considerations, e.g., by customary comparison of the differential activities of the subject compounds and of a known agent, e.g., by means of an appropriate, conventional pharmacological protocol.

### Methods of Inhibiting Matrix Metalloproteinases (MMPs) Using PL Conjugates

In another embodiment, provided herein a method for inhibiting a MMP production in a cell, comprising contacting the cell with a composition comprising a compound of the invention. In another embodiment, provided herein a method for inhibiting MMP 2 production in a cell, comprising contacting the cell with a composition comprising a compound of the invention. In another embodiment, provided herein a method for inhibiting MMP 9 production in a cell, comprising contacting the cell with a composition comprising a compound of the invention. In another embodiment, provided herein a method for inhibiting a MMP production in a malignant cell, comprising contacting the cell with a composition comprising a compound of the invention. In another embodiment, provided herein a method for inhibiting a MMP production in a cell expressing elevated level of a MMP, comprising contacting the cell with a composition comprising a compound of the invention.

In another embodiment, provided herein a method for inhibiting invasiveness of a cancer cell, comprising the step of contacting a cancer cell with a composition comprising a compound of the invention. In another embodiment, provided herein a method for inhibiting the production of a MMP in a cell, comprising the step of contacting the cancer cell with a composition comprising a compound as described herein. In another embodiment, provided herein a method for inhibiting the production of a MMP in a cancer cell, comprising the step of contacting the cancer cell with a composition comprising a compound as described herein. In another embodiment, provided herein a method for inhibiting the production of a MMP in a cell, comprising the step of contacting the cell with a composition comprising a compound as described herein. In another embodiment, provided herein a method for inhibiting the expression of a MMP in a cell, comprising the step of contacting the cell with a composition comprising a compound as described herein. In another embodiment, provided herein a method for inhibiting the transcription of a MMP in a cell, comprising the step of contacting the cell with a composition comprising a compound as described herein. In another embodiment, provided herein a method for inhibiting the activation of a pre-MMP in a cell, comprising the step of contacting the cell with a composition comprising a compound as described herein.

In another embodiment, provided herein a method for inhibiting a collagenolytic activity of a MMP, comprising the step of contacting a MMP with a composition comprising a compound as described herein. In another embodiment, provided herein a method for inhibiting a collagenolytic activity of a MMP, comprising the step of contacting a metastatic cell with a composition comprising a compound as described herein. In another embodiment, provided herein a method for inhibiting a collagenolytic activity of a MMP, comprising the step of contacting an endothelial cell with a composition comprising a compound as described herein. In another embodiment, provided herein a method for inhibiting a collagenolytic activity of a cell by contacting the cell with a MMP inhibitor as described herein. In another embodiment, provided herein a method for inhibiting a collagenolytic activity of an endothelial cell by contacting the cell with a MMP inhibitor as described herein. In another embodiment, provided herein a method for inhibiting a collagenolytic activity of a malignant cell by contacting the cell with a MMP inhibitor as described herein.

In another embodiment, provided herein a method for treating a subject afflicted with a disease in which increased production of a MMP is associated with the disease, comprising the step of administering to the subject a composition comprising a PL compound as described hereinabove. In another embodiment, provided herein a method for treating a subject afflicted with a disease in which increased activity of a MMP is associated with the disease, comprising the step of administering to the subject a composition comprising a PL compound as described hereinabove. In another embodiment, provided herein a method for treating a subject suffering from a medical condition in which increased production of a MMP causes the medical condition, comprising the step of administering to the subject a composition comprising a PL compound as described hereinabove. In another embodiment, provided herein a method for treating a subject suffering from a medical condition in which increased activity of a MMP causes the medical condition, comprising the step of administering to the subject a composition comprising a PL compound as described hereinabove. In another embodiment, provided herein a method for treating a subject suffering from a medical condition in which increased production of a MMP is associated with the medical condition, comprising the step of administering to the subject a composition comprising a PL compound as described hereinabove. In another embodiment, provided herein a method for treating a subject suffering from a medical condition in which increased activity of a MMP is associated with the medical condition, comprising the step of administering to the subject a composition comprising a PL compound as described hereinabove.

In another embodiment, a medical condition or a disease treatable by the compounds of the invention is characterized by excessive MMP activity and/or production. In another embodiment, the medical condition or disease is selected from: Pterygium, Kerataconus, macular degeneration, corneal melting, occlusions in the choroid, a heart disease, arthritis, a cerebral disease, a tissue ulceration, abnormal wound healing, a periodontal disease, a bone disease, a cancer characterized by tumor growth, a cancer characterized by tumor metastasis or invasion, HIV-infection, decubitus, decubitis ulcer, restenosis, epidermolysis bullosa, sepsis, septic shock, neoplasm, psoriasis, neovascularization, a liver disease, or multiple sclerosis.

In another embodiment, the medical condition or disease is selected from: abnormal wound healing, acne, acute coronary syndrome, acute infection, AIDS, alcoholism, allergic conjunctivitis, allergic reactions, allergic rhinitis, ALS, Alzheimer's diseases, anaphylaxis, aneurysmal aortic disease, angina, angiofibromas, anorexia, aortic aneurysm, ARDS, aspirin-independent anti-thrombosis, asthma, atherosclerosis, atherosclerotic plaque rupture, atopic dermatitis, benign hyperplasia, bleeding, bone fractures, bronchitis, burns, cachexia, cancer, cardiac infarction, cardiac insufficiency, cardiomyopathy, cerebral hemorrhaging, cerebral ischemia, cerebral vascular dementia, CHF, chronic bronchitis, chronic dermal wounds, chronic obstructive pulmonary disease, cirrhosis, congestive heart failure, corneal injury, coronary thrombosis, Crohn's disease, cystic fibrosis, decubitis ulcer, diabetic peripheral neuropathy, diabetic retinopathy, diabetic ulcers, Duchenne's muscular dystrophy, emphysema, endometriosis, endosclerosis, epidermolysis bullosa, eye disorders, fibrosis, gastritis, gingivitis, glomerular diseases, glomerulonephritis, gout, graft rejection, gum disease, GVHD, Hashimoto's thyroiditis, head trauma, headaches, heart attacks, heart failure, hemangiomas, hemorrhage, hepatitis, hirsutism, Huntington's disease, hypertension, insulin resistance, interstitial nephritis, ischemia, ischemic heart disease, Kaposi's sarcoma, keratinization, keratitis, kidney failure, leishmaniasis, leprosy, leukemia, leukocyte infiltration, liver cirrhosis, loss of appetite, macular degeneration, malaria, mandibular joint disease, memory impairment, meningitis, migraine, miscarriage, multi-infarct dementia, multiple sclerosis, muscular dystrophy, myalgia, myasthenia gravis, myelinic degradation, myocardial infarction, myopia, neovascular glaucoma, neuroinfalmmation, ocular tumors, optic neuritis, osteoarthritis, osteopenia, Paget's disease, pain, pancreatitis, Parkinson's disease, periodontitis, peripheral vascular disease, polyarteritis nodositas, polychondritis, premature childbirth, premature rupture of fetal membranes, prion disease, proliferative retinopathies, proteinurea, pseudogout, psoriasis, pterygium, pulmonary emphysema, radiation damage, rattle snake bite, Reiter's syndrome, renal fibrosis, reocclusion, reperfusion injury, restenosis, scleritis, scleroderma, senile dementia, senility, sepsis, septic shock, Sharp syndrome, Sjoegren's syndrome, SLE, spondylosis, stenosis, sterility, stroke, system sclerosis, thrombosis, toxic effects of chemotherapy, toxic shock, tuberculosis, corneal ulcerations, epidermal ulcerations, gastric ulcerations, ulcertive colitis, uremia, vasculitis, ventricular dilation, vesicular epidermolysis.

In another embodiment, the medical condition or disease is selected from: osteoarthritis, rheumatoid arthritis, inflammatory enteropathy, Crohn's disease, emphysema, acute dyspnea syndrome, asthma, chronic obstructive disease, acute bronchitis, bronchitis, Alzheimer's disease, transplanting toxicity, cachexia, allergic reaction, allergic contact anaphylaxis, allergic conjunctive, allergic rhinitis, solid cancer such as but not limited to colon cancer, breast carcinoma, lung cancer, prostata carcinoma, malignant hemapoiesis such as but not limited to leukemia and lymphoma, restonosis, periodontis, eoidermolysis bulla, osteoporosis, loosening of artificial joint implants, atherosclerosic local laceration, athermanous placoido cleavage, aortic aneurysm, abdominal aneurysm, cerebral aortic aneurysm, congestive heart failure, myocardial infarction, seizure, cerebral ischemia, caput injury, myelon injury, neurodegenerative disease (acute and chronic), autoimmunity disease, Huntington disease, Parkinsonism, migraine, depression, peripheral neuropathy, pain, cerebral amyloidal avasculopathy, nootropic or performance intensity, amyotrophic lateral sclerosis, multiple sclerosis, eyepiecevasculogenesis, corneal injury, macula retinal degeneration, unusual wound healing, burn, diabetes, diabetic peripheral neuropathy, diabetic retinitis, diabetic ulcer, tumor infiltration, tumor growth, tumor metastasis, epicauma (macula), pleurisy, AIDS, sepsis, septic shock, contusion, acute infection, alcoholism, ALS, anaphylaxis, angina, hemangiofibroma, anorexia, ARDS, aspirin independent antithrombosisi, atopic dermatitis, benign vegetation, bleeding, fracture, burn, cachexia, myocardosis, cerebral apoplexy, cerebral angio dementia, CHF, chronic dermato wound, coronary thrombosis, cystic fibrosis, decubitis ulcer, Duchene's myodystorophy, emphysema, endometriosis, epidermolysis, oculopathy, fibrosis, gastritis, glomerulitis, glomerular nephritis, gout, transplantation rejection, disease of gums, GVHD, Hashimoto's disease, caput injury, head ache, angioma, hepatitis, trichauxis, hypertension, insulin resistance, spacial nephritis, ischemia, ischemic malum cordis, Kaposis sarcoma, cornification, keratitis, renal insufficiency, leishmaniasis, leprosy, leukemia, leukocyte infiltration, hepatocirrhosis, malaria, lower jaw temporomandibular arthritis, dysmnesia, meningitis, migraine, abortion, multiple cerebral infarction dementia, myodystrophy, muscle pain, myasthenia gravis, myelinosis, cardiac infarction, myopia, neovascular glaucoma, neuritis, carcinoma of eye, fasciculitis, Paget's disease, pain, pancreatis, Parkinsonism, periodontitis, peripheral disease, polyarteritis nodosa, polychondritis, premature birth, embryo membrane dehiscence, prion disease, retinitis proliferans, protein urea, pseudo gout, psoriasis, pterigium, pulmonary emphysema, radiation obstacles, rattle snake morsus (bite), Reiter's syndrome, renal fibrosis, distal occlusion, recurrent disorder, restenosis, scleritis, scleroderma, senile dementia, senility, septis, septic shock, Sharp-syndrome, Sjogren's syndrome, SLE, spondylolysis, stegnosis, infertility, seizure, thrombostasis, toxicity by chemotherapy, toxic shock, tuberculosis, uremia, vasculitis, ventricle dilation, epidermolysis bullosa and, any other diseases or medical conditions known to one of skill in the art as specified by overexpression of metalloproteinase.

In another embodiment, an inhibitor as described herein inhibits a MMP and thus acting as an immunosuppressant. In another embodiment, an inhibitor as described herein inhibits a MMP and thereby inhibits the activity of TNF-α and/or IFN-γ production. In another embodiment, an inhibitor as described herein inhibits a soluble MMP.

In another embodiment, a MMP inhibitor as described herein inhibits localized degradation of existing ECM. In another embodiment, a MMP inhibitor as described herein inhibits cytoskeletal rearrangement. In another embodiment, an inhibitor as described herein inhibits cell translocation. In another embodiment, a MMP inhibitor as described herein inhibits cleavage of collagen. In another embodiment, a MMP inhibitor as described herein inhibits cleavage of gelatin. In another embodiment, an inhibitor as described herein inhibits a MMP in a fibroblasts, a PNL, a macrophage, a Keratinocyte, an EC, a T-cell, or an eosinophil. In another embodiment, a MMP inhibitor as described herein, inhibits the production of IL1, IL10, TNF-α, TGF, FGF, PDGF, or any combination thereof.

In another embodiment, a MMP inhibitor as described herein is administered to a subject having hi MMP levels in the blood. In another embodiment, a MMP inhibitor as described herein is administered to a subject having MMP levels at above a threshold level in the blood. In another embodiment, a MMP inhibitor as described herein is administered to a subject having above normal MMP levels in the blood. In another embodiment, a MMP inhibitor as described herein is administered to a subject having hi MMP levels in the urine. In another embodiment, a MMP inhibitor as described herein is administered to a subject having MMP levels at above a threshold level in the urine. In another embodiment, a MMP inhibitor as described herein is administered to a subject having above normal MMP levels in the urine.

In another embodiment, a MMP is a Zn²⁺ endopeptidase. In another embodiment, a MMP is a 92 kDa gelatinase, collagenase, stromelysin or a membrane-bound MMP. In another embodiment, a MMP is expressed in an inflammatory condition. In another embodiment, a MMP is capable of degrading a connective tissue. In another embodiment, a MMP is a gelatinase such as MMP-2 and MMP-9. In another embodiment, a MMP is a stromelysin such as MMP-3. In another embodiment, a MMP is a collagenase such as MMP-1, MMP-8, and MMP-13 which are involved in tissue matrix degradation and have been implicated in many pathological conditions involving abnormal connective tissue and basement membrane matrix metabolism.

In another embodiment, a MMP is a proteolytic enzyme. In another embodiment, a MMP maintains the integrity of the extracellular matrix. In another embodiment, excessive MMP activity results in loss of structural proteins that maintain the normal architecture of an organ. In another embodiment, excessive MMP activity results in activation of inflammatory cells that perpetuate organ damage. In another embodiment, a MMP activates an acute inflammatory pathway. In another embodiment, a MMP activates a chronic inflammatory pathway, involved in liver damage. Second, these enzymes are especially highly expressed in a variety of liver diseases. In another embodiment, a MMP is involved in maintaining the structural integrity of an organ. In another embodiment, a MMP is involved in the progression of fibrogenesis.

In another embodiment, a MMP inhibitor (PL of the invention) as described herein is used to control excessive proteolytic degradation of the extracellular matrix. In another embodiment, a MMP inhibitor (PL of the invention) as described herein is used to control cell invasion.

In another embodiment, a MMP inhibitor as described herein is selective to MMP-2 and/or MMP-9. In another embodiment, a MMP inhibitor as described herein is not selective to a particular MMP. In another embodiment, arachidonic acid (AA)-derived metabolites regulates MMP expression. In another embodiment, phospholipase A₂, the AA producing enzymes, regulates MMP expression.

In another embodiment, over expression pattern of MMP 2, MMP 9, MMP 13, or a combination thereof leads to the progression of liver damage. In another embodiment, a MMP inhibitor as described herein inhibits the progression of liver damage caused by excessive activity of MMP 2, MMP 9, MMP 13, or a combination thereof. In another embodiment, a MMP inhibitor as described herein inhibits the progression of liver damage caused by excessive activity of MMP 2, MMP 9, MMP 13, or a combination thereof in activated stellate cells. In another embodiment, a MMP inhibitor as described herein inhibits the progression of liver damage caused by excessive activity of MMP 2, MMP 9, MMP 13, or a combination thereof in activated Kupffer cells. In another embodiment, a MMP inhibitor as described herein ameliorates symptoms associated with a liver disease. In another embodiment, a MMP inhibitor as described herein ameliorates symptoms associated with liver damage. In another embodiment, a MMP inhibitor as described herein is used in combination another compound or compounds which induce mechanisms of hepatoprotection. In another embodiment, a MMP inhibitor as described herein is used in combination another compound or compounds which induce mechanisms of hepatogeneration.

In another embodiment, a MMP inhibitor as described herein is useful for the treatment of diseases related to bone or cartilage, such as rheumatoid arthritis, osteoarthritis, etc. In another embodiment, a MMP inhibitor as described herein is useful for inhibiting the loss of glycoprotein and collagen in articular cartilage.

In another embodiment, a MMP inhibitor as described herein is useful for preventing arteriosclerosis. In another embodiment, a MMP inhibitor as described herein is useful for inhibiting the progress of arteriosclerosis. In another embodiment, a MMP inhibitor as described herein is useful in treating a subject afflicted with arteriosclerosis.

In another embodiment, a MMP inhibitor as described herein is useful for preventing re-stricturization (re-stenochoria) of post angiopoietic operation. In another embodiment, a MMP inhibitor as described herein is useful for inhibiting the progress of re-stricturization of post angiopoietic operation. In another embodiment, a MMP inhibitor as described herein is useful in treating a subject afflicted with re-stricturization (re-stenochoria) of post angiopoietic operation.

In another embodiment, a MMP inhibitor as described herein is useful as an etiomatic therapy. In another embodiment, a MMP inhibitor as described herein is a MMP 13 inhibitor. In another embodiment, a MMP inhibitor as described herein is useful for preventing bone arthritis and rheumatoid arthritis. In another embodiment, a MMP inhibitor as described herein is useful for inhibiting the progress of bone arthritis and rheumatoid arthritis. In another embodiment, a MMP inhibitor as described herein is useful in treating a subject afflicted with bone arthritis and/or rheumatoid arthritis.

In another embodiment, a MMP inhibitor as described herein is useful as a prophylactic and/or therapeutic treating agent.

In another embodiment, a MMP inhibitor as described herein is used for inhibiting invasion and metastasis of malignant cells. In another embodiment, a MMP-2 and/or MMP-9 inhibitor as described herein is used for inhibiting invasion and metastasis of malignant cells. In another embodiment, a MMP-2 and/or MMP-9 inhibitor as described herein is used for inhibiting hematological malignancies. In another embodiment, a MMP inhibitor as described herein is used for treating a subject afflicted with acute myeloid leukemia. In another embodiment, a MMP inhibitor as described herein is used for treating a subject afflicted with acute myelomonocytic leukemia. In another embodiment, a MMP inhibitor as described herein is used for treating a subject afflicted with acute monoblastic and monocytic leukemia. In another embodiment, a MMP inhibitor as described herein is used for treating a subject afflicted with acute erytroid leukemia. In another embodiment, a MMP inhibitor as described herein is used for treating a subject afflicted with acute megakaryoblastic leukemia. In another embodiment, a MMP inhibitor as described herein is used for treating a subject afflicted with acute basophilic leukemia. In another embodiment, a MMP inhibitor as described herein is used for treating a subject afflicted with acute panmyelosis with myelofibrosis. In another embodiment, a MMP inhibitor as described herein is used for treating a subject afflicted with myeloid sarcoma.

In another embodiment, a MMP inhibitor as described herein is used for treating a subject afflicted with a hernia. In another embodiment, a MMP inhibitor as described herein is used for treating a subject afflicted with an abdominal hernia. In another embodiment, a MMP inhibitor as described herein is used for treating a subject afflicted with a groin hernia. In another embodiment, a MMP inhibitor as described herein is used for reducing the risk of recurrent hernias.

In another embodiment, a MMP inhibitor as described herein is used for treating a subject afflicted with lymphangioleiomyomatosis. In another embodiment, a MMP inhibitor as described herein inhibits tissue degradation in patients with lymphangioleiomyomatosis.

In another embodiment, a MMP inhibitor as described herein is used for treating a subject suffering from pseudocyst formation. In another embodiment, a MMP inhibitor as described herein is used for treating a subject suffering from an accumulation of oedema. In another embodiment, a MMP inhibitor as described herein is used for treating a subject afflicted with sinusitis. In another embodiment, a MMP inhibitor as described herein is used for treating a subject afflicted with chronic sinusitis. In another embodiment, a MMP inhibitor as described herein is used for treating a subject afflicted with nasal polyposis.

In another embodiment, a MMP inhibitor as described herein is used for treating a subject afflicted with multiple sclerosis. In another embodiment, a MMP inhibitor as described herein is used for ameliorating symptoms associated with multiple sclerosis in a subject in need thereof.

In another embodiment, a MMP inhibitor as described herein is used for treating a child afflicted with an inflammatory condition which disrupts the elastic lamina. In another embodiment, a MMP inhibitor as described herein is used for treating a subject afflicted with Kawasaki disease. In another embodiment, a MMP inhibitor as described herein is used for treating a subject afflicted with an acute type of systemic vasculitis in children.

In another embodiment, a subject according to the invention is a human subject. In another embodiment, a subject according to the invention is a mammal. In another embodiment, a subject according to the invention is a non-human mammal. In another embodiment, a subject according to the invention is a farm animal. In another embodiment, a subject according to the invention is a primate. In another embodiment, a subject according to the invention is a pet. In another embodiment, a subject according to the invention is a laboratory animal.

In another embodiment, a MMP inhibitor as described herein is used for treating a subject afflicted with a heart disease. In another embodiment, a MMP inhibitor as described herein is used for treating a subject afflicted with hypertension. In another embodiment, a MMP inhibitor as described herein is used for treating a subject afflicted with myocardial fibrosis.

In another embodiment, a MMP inhibitor as described herein is used for treating a subject afflicted with psoriasis. In another embodiment, a MMP inhibitor as described herein is used for treating a subject afflicted with cutaneous psoriasis. In another embodiment, a MMP inhibitor as described herein is used for treating a subject afflicted with psoriatic arthritis. In another embodiment, a MMP inhibitor as described herein is used for treating a subject afflicted with a skin lesion.

In another embodiment, a MMP inhibitor as described herein inhibits the expression of a MMP. In another embodiment, a MMP inhibitor as described herein inhibits the transcription of a MMP. In another embodiment, a MMP inhibitor as described herein inhibits activation of a MMP post-transcriptionally. In another embodiment, a MMP inhibitor as described herein inhibits the activation of a MMP proenzyme. In another embodiment, a MMP inhibitor as described herein inhibits the collagenolytic activity of a MMP. In another embodiment, a MMP inhibitor as described herein inhibits the collagenolytic activity of a cell. In another embodiment, a MMP inhibitor as described herein inhibits the collagenolytic activity of a cancer cell. In another embodiment, a MMP inhibitor as described herein inhibits the collagenolytic activity of a metastatic cell. In another embodiment, a MMP inhibitor as described herein inhibits the collagenolytic activity of a tumor cell. In another embodiment, a MMP inhibitor as described herein inhibits the metastatic potential of a solid tumor. In another embodiment, a MMP inhibitor as described herein inhibits a MMP in stromal cells. In another embodiment, a MMP inhibitor as described herein inhibits a neovascularizationiangiogenesis. In another embodiment, a MMP inhibitor as described herein inhibits lysis of matrix surrounded by endothelial cells thus enabling the inhibiting the invasion of new vascular structures into a tissue. In another embodiment, a MMP inhibitor as described herein inhibits lysis of matrix surrounded by endothelial cells thus enabling the inhibiting the invasion of new vascular structures into a malignant tissue.

In another embodiment, a MMP inhibitor as described herein reduces the invasive and metastatic potential of tumor cells. In another embodiment, a MMP inhibitor as described herein blocks the invasive activity of cancer cells such as prostate cancer cells. In another embodiment, a MMP inhibitor as described herein inhibits the degradation of ECM by melanoma cells.

In another embodiment, the invention provides a method of treating a subject afflicted with a metastatic cancer, comprising the step of administering to the subject a composition comprising a compound of the invention for inhibiting a MMP. In another embodiment, the invention provides a method of treating a subject afflicted with a metastatic cancer, comprising the step of administering to the subject a composition comprising a compound of the invention for inhibiting MMP 2 and/or MMP 9. In another embodiment, the invention provides a method of treating a subject afflicted with a metastatic cancer, comprising the step of administering to the subject a composition comprising a compound of the invention for inhibiting a MMP in a cancerous cell. In another embodiment, the invention provides a method of treating a subject afflicted with a metastatic cancer, comprising the step of administering to the subject a composition comprising a compound of the invention for inhibiting a MMP in a malignant cell.

In another embodiment, a MMP inhibitor as described herein inhibits MMP-9. In another embodiment, a MMP inhibitor as described herein inhibits MMP in trophoblasts, osteoclasts, leukocytes, and their precursors. In another embodiment, a MMP inhibitor as described herein counteracts the activity of growth factors, cytokines, cell-cell and cell-ECM adhesion molecules which induce MMP production and/or activation. In another embodiment, a MMP inhibitor as described herein inhibits a MMP metabolite. In another embodiment, a MMP inhibitor as described herein inhibits invasion of cells through matrix barriers and collagenolysis during invasion and tumor progression.

In some embodiments, the compounds of this invention are useful in any application in which neoplasia or carcinogenesis is halted, modulated or altered in any way that is beneficial to a subject in need.

In some embodiments, this invention provides for the use of a compound of formula I-XXI, or any compound as herein described, or its prodrug, analog, isomer, metabolite, derivative, pharmaceutically acceptable salt, pharmaceutical product, polymorph, crystal, impurity, N-oxide, hydrate or any combination thereof, for treating, reducing the severity of, reducing the incidence of, or reducing pathogenesis of neoplasia or carcinogenesis in a subject. In another embodiment, the neoplasia comprises adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, ewings family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, small cell lung cancer, non small cell lung cancer, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metasatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor, or any combination thereof.

In some embodiments, this invention provides the use of a compound of formula I-XXI, or any compound as herein described, including an analog, derivative, isomer, metabolite, pharmaceutically acceptable salt, pharmaceutical product, polymorph, crystal, impurity, hydrate, N-oxide or any combination thereof, for treating, reducing the severity of, reducing the incidence of, or reducing pathogenesis of cancer. In another embodiment, the cancer comprises any cancer of soft tissue. In one embodiment the cancer comprises prostate cancer; bladder cancers; brain cancers; bone tumors, colon cancer, endometrial cancer, liver cancer, lung cancer, lymphatic cancer, kidney cancer, osteosarcoma cancer, ovarian cancer, pancreas cancer, penis cancer, skin cancer, thyroid cancer; and/or hormone-dependent cancers.

In another embodiment, the subject is male. In another embodiment, the subject is female. In some embodiments, while the methods as described herein may be useful for treating either males or females, females may respond more advantageously to administration of certain compounds, for certain methods, as described and exemplified herein.

In another embodiment, the subject suffers from a sarcoma. In another embodiment, the subject suffers from an adenocarcinoma, colon carcinoma, melanoma, breast carcinoma, leukemia, lymphoma, gastric carcinoma, glioblastoma, astrocytoma, bladder carcinoma, pleural mesothelioma, oat cell carcinoma or bronchogenic carcinoma. In another embodiment, “treating” refers to both therapeutic treatment and prophylactic or preventative measures, wherein the object is to prevent or lessen the targeted pathologic condition or disorder as described hereinabove. Thus, in another embodiment, treating may include suppressing, inhibiting, preventing, treating, or a combination thereof. Thus, In another embodiment, “treating” refers inter alia to increasing time to sustained progression, expediting remission, inducing remission, augmenting remission, speeding recovery, increasing efficacy of or decreasing resistance to alternative therapeutics, or a combination thereof. In another embodiment, “preventing” refers inter alia to delaying the onset of symptoms, preventing relapse to a disease, decreasing the number or frequency of relapse episodes, increasing latency between symptomatic episodes, or a combination thereof. In another embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In another embodiment, the terms “treating” or “treatment” includes preventative as well as disorder remitative treatment. The terms “reducing”, “suppressing” and “inhibiting” have their commonly understood meaning of lessening or decreasing, in another embodiment, or delaying, in another embodiment, or reducing, in another embodiment the incidence, severity or pathogenesis of a disease, disorder or condition. In embodiment, the term treatment refers to delayed progression of, prolonged remission of, reduced incidence of, or amelioration of symptoms associated with the disease, disorder or condition. In another embodiment, the terms “treating” “reducing”, “suppressing” or “inhibiting” refer to a reduction in morbidity, mortality, or a combination thereof, in association with the indicated disease, disorder or condition. In another embodiment, the term “progression” refers to an increasing in scope or severity, advancing, growing or becoming worse. The term “recurrence” means, in another embodiment, the return of a disease after a remission. In another embodiment, the methods of treatment of the invention reduce the severity of the disease, or in another embodiment, symptoms associated with the disease, or in another embodiment, reduces the number of biomarkers expressed during disease.

In another embodiment, the term “treating” and its included aspects, refers to the administration to a subject with the indicated disease, disorder or condition, or in some embodiments, to a subject predisposed to the indicated disease, disorder or condition. The term “predisposed to” is to be considered to refer to, inter alia, a genetic profile or familial relationship which is associated with a trend or statistical increase in incidence, severity, etc. of the indicated disease. In some embodiments, the term “predisposed to” is to be considered to refer to inter alia, a lifestyle which is associated with increased risk of the indicated disease. In some embodiments, the term “predisposed to” is to be considered to refer to inter alia, the presence of biomarkers which are associated with the indicated disease, for example, in cancer, the term “predisposed to” the cancer may comprise the presence of precancerous precursors for the indicated cancer.

In some embodiments, the term “reducing the pathogenesis” is to be understood to encompass reducing tissue damage, or organ damage associated with a particular disease, disorder or condition. In another embodiment, the term “reducing the pathogenesis” is to be understood to encompass reducing the incidence or severity of an associated disease, disorder or condition, with that in question. In another embodiment, the term “reducing the pathogenesis” is to be understood to encompass reducing the number of associated diseases, disorders or conditions with the indicated, or symptoms associated thereto.

The term “administering”, in another embodiment, refers to bringing a subject in contact with a compound of the present invention. Administration can be accomplished in vitro, i.e. in a test tube, or in vivo, i.e. in cells or tissues of living organisms, for example humans. In another embodiment, the present invention encompasses administering the compounds of the present invention to a subject.

In another embodiment, symptoms being treated are primary, while in another embodiment, symptoms are secondary. In another embodiment, “primary” refers to a symptom that is a direct result of neoplasia or carcinogenesis, while in another embodiment, “secondary” refers to a symptom that is derived from or consequent to a primary cause. In another embodiment, the compounds for use in the present invention treat primary or secondary symptoms or secondary complications related to neoplasia or carcinogenesis. In another embodiment, the compounds for use in the present invention treat primary or secondary symptoms or secondary complications related to neoplasia or carcinogenesis.

In another embodiment, “symptoms” may be any manifestation of a disease or pathological condition, comprising inflammation, swelling, fever, pain, bleeding, itching, runny nose, coughing, headache, migraine, dizziness, blurry vision, diarrhea, vomiting, constipation, gas, indigestion, etc.

Thus, in one embodiment of the present invention, the compounds for use in the present invention are directed towards the resolution of symptoms of a disease or disorder of neoplasia or carcinogenesis. In another embodiment, the compounds affect the pathogenesis underlying neoplasia or carcinogenesis.

In another embodiment, neoplasia or carcinogenesis may affect a cell, in another embodiment, a vertebrate cell, in another embodiment, a mammalian cell, and in another embodiment, a human cell. It is to be understood that compounds of the present invention may be efficacious in treating any cell type in which neoplasia or carcinogenesis is present or in which the causes of neoplasia or carcinogenesis may exert an effect. In another embodiment, a compound for use in the present invention may localize to or act on a specific cell type. In another embodiment, a compound for use in the present invention may be cytoprotective. In one embodiment a compound for use in the present invention may be inserted or partially inserted into a cell membrane. In another embodiment a compound for use in the present invention may be effective in treating a plurality of cell types.

In one embodiment of the present invention, the useful pharmacological properties of the compounds for use in the present invention, some of which are described hereinabove, may be applied for clinical use, and disclosed herein as methods for the prevention or treatment of a disease. The biological basis of these methods may be readily demonstrated by standard cellular and animal models of disease.

In another embodiment, the pharmacological activities of compounds for use in the present invention, including membrane stabilization, anti-inflammation, anti-oxidant action, and attenuation of chemokine levels, may contribute to a treated cell's resistance to neoplasia or carcinogenesis. In another embodiment, cell membrane stabilization may ameliorate or prevent tissue injury arising in the course of an intestinal disease. In another embodiment, anti-oxidant action may limit oxidative damage to cell and blood components arising in the course of an intestinal disease. In another embodiment, attenuation of chemokine levels may attenuate physiological reactions to stress that arise in the course of an intestinal disease.

In one embodiment of the invention, the compounds for use in the present invention described herein can be used to treat disease, through amelioration, or prevention, of tissue injury arising in the course of pathological disease states by stabilizing cell membranes; limiting oxidative damage to cell and blood components; or attenuating physiological reactions to stress, as expressed in elevated chemokine levels.

In another embodiment, methods of the present invention involve treating a subject by inter alia controlling the expression, production, and activity of phospholipases such as PLA2; controlling the production and/or action of lipid mediators, such as eicosanoids, platelet activating factor (PAF) and lyso-phospholipids; amelioration of damage to cell surface glycosaminoglycans (GAG) and proteoglycans; controlling the production of oxidants, oxygen radicals and nitric oxide; protection of cells, tissues, and plasma lipoproteins from damaging agents, such as reactive oxygen species (ROS) and phospholipases; controlling the expression, production, and activity of cytokines, chemokines and interleukins; anti-oxidant therapy; anti-endotoxin therapy or any combination thereof.

In one embodiment of the invention, the term “controlling” refers to inhibiting the production and action of the above mentioned factors in order to maintain their activity at the normal basal level and suppress their activation in pathological conditions.

It will be appreciated by one skilled in the art that the compounds characterized by the structures (A), (I), (II), (III), (IV), (V), (VI), (VII), (VII), (IX), (IXa), (IXb), (X), (XI), (XII), (XIII), (XIV), (XV), (XVI), (XVII), (XVIII), (XIX), (XX), (XXI), (XXII), or any combination thereof may be administered according to any regimen, at any dosage, to suit a particular application, for example cancer type or cancer stage, or a particular subject, for example, male versus female, or for example, in consideration of the age and lifestyle choice of the subject. In some embodiments, such varied regimens are a function of the presence of preneoplastic lesions or frank neoplasia, or in some embodiments, the occurrence of metastasis.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

### Compounds

Porcine pancreatic and Crotalus atrox PLA₂s were purchased from Sigma-Aldrich, St. Louis, Mo., USA. Hyaluronic acid-N-conjugated phosphatidyl-ethanolamine (HyPE, M.W. cr. 50 kD), was synthesized in the laboratory of S. Yedgar (Dan, P., et al. Biochemistry, 37, 6199-204 (1998); Beck, G., et al. Crit. Care Med, 31, 2015-21 (2003); Offer, S., et al. Am J Physiol Lung Cell Mol Physiol, 288, L523-9 (2005)).

### Cell Culture

Human fibrosarcoma HT-1080 cells (CCL 121, ATCC, Rockville Md.) were maintained in DMEM supplemented with calf serum, 10%. Glutamine, pyruvate, non-essential amino acids, vitamins and antibiotics (Biological Industries, Kibbutz Beth HaEmek, Israel) were added as additional supplements.

### Determination of Basement Membrane Invasiveness

Boyden chamber chemoinvasion assays were performed as previously described (Reich, R., M. et al. Clin Exp Metastasis, 13, 134-40 (1995)). Matrigel (reconstituted basement membrane; 25 microgram) was dried on a polycarbonated filter (Nucleopore® Polyester PVP free; Whatman International Ltd., UK). Fibroblast conditioned medium (obtained from confluent NIH-3T3 cells cultured in serum free DMEM) is used as the chemoattractant. Cells were harvested by brief exposure to 1 mM EDTA, washed with DMEM with 5 microgram collagen IV instead of Matrigel. This amount of collagen does not form a barrier to the migrating cells but rather an attachment substratum, and thus serves to measure cell motility.

### Determination of MMP Activity (Zymography)

Sub-confluent cell cultures were incubated for 6/24 h in serum-free DMEM and the resulted supernatant was analyzed for collagenolytic activity. The collagenolytic activity was determined on a gelatin impregnated (1 mg/ml, Difco, USA), SDS-PAGE 8% gel, as previously described (Brassart, B., A. et al. Clin Exp Metastasis, 16, 489-500 (1998)). Containing 0.1% BSA, and added to the Boyden chambers (200,000 cells). The chambers were incubated at 37° C. in humidified atmosphere of 5% CO₂/95% air for 6 h. The cells have traversed the Matrigel layer and attached to the lower surface of the filter and stained with Diff Quick (Dade Diagnostics, USA) and counted in five random fields. The mean of the counts was calculated and values are expressed in terms non-treated HT-1080 cells normalized to 100%.

### Determination of Cell Chemotaxis

To rule out the possibility that the used inhibitors affect cell motility, chemotaxis evaluation was performed in a similar way to basement membrane invasion, with the exception that the filters are coated. The bands were scanned (Epson Perfection 3200 Photo), and the intensity was determined with the NIH image 1.63 software. All values are expressed in terms in of untreated HT-1080 cells divided by the absorbance of the 3-(4,5-dimethylthiazol-2-yl)-2,5-diphenyltetrazolium bromide viability assay (MTT) [Kudo, I. & M. Murakami. Prostaglandins Other Lipid Mediat, 68-69, 3-58 (2002)] normalized to 100%.

### Determination of Cell PLA₂ Activity

Confluent HT-1080 cells were metabolically labeled with either [³H-AA] or [³H-OA](0.5 microCi/24 well plate) (Amersham Biosciences, UK), by overnight incubation with the radioactive fatty acid, then washed and the temporal release of the labeled fatty acid to the culture medium was monitored under the different treatments (Dan, P., et al. FEBS Lett, 383, 75-8 (1996).

### Determination of Exogenous PLA₂ Activity

Lipolytic activity of exogenous PLA₂ was determined by using 4N3OBA (4-nitro-3-hydroxy-benzoic acid) (Sigma-Aldrich, St. Louis, Mo., USA) as a substrate. 10 microliter of PLA_{2 [}1 u/ml and 0.5 u/ml] in Tris-HCl (pH=8 100 nM) was incubated with 190 microliter substrate solution (4N3OBA resuspended in 150 mM KCl, 10 mM CaCl₂, 50 mM Tris-HCl, pH=7.5) at room temperature for 1 h. The PLA₂ activity calculated as: (A_{425 nm}−A_{600 nm})[OD₄₂₅/h]×0.07862 [micromol/OD_{425 nm}]×(1/sample volume [1/ml]).

### Identification of Cell sPLA₂ and sPLA₂ Receptor Expression

Cultured HT-1080 cells were assessed for the expression of mRNA for sPLA₂s. Total RNA was isolated from the cells using Tri-reagent (Sigma-Aldrich, St. Louis, Mo., USA). First strand cDNA was transcribed with M-MLV reverse transcriptase (RT) (Promega, Madison, USA). Each cDNA (5 microgram) was amplified in standard PCR reaction (30-35 cycles) containing ReddyMix™ Master Mix (1.5 mM MgCl₂) (ABgene®, UK) and 1.5 mM oligonucleotide primers. The PCR was carried out in an Eppendorf Mastercycler with an initial 5 min denaturing at 94° C., followed by the sequence of denaturation (95° C., 30 s), annealing (50° C., 30 s), and extension (72° C., 2 min). A final extension of 20 min at 72° C. ended the reaction.

PCR analysis was performed on reversed transcribed mRNA using 5′CTT-GAC-TGC-AAG-ATG-AAA-CTC (SEQ ID NO: 1) as sense and 5′CTG-ACA-ATA-CTT-CTT-GGT-GTC (SEQ ID NO: 2) as antisense primers for sPLA₂-IB to give a 455 bp; 5′ACC-ATG-AAG-ACC-CTC-CTA-CT (SEQ ID NO: 3) as sense and 5′gaa-gag-ggg-act-cag-caa-cg (SEQ ID NO: 4) as antisense primers for sPLA₂-IIA to give a 449 bp; 5′CAG-GGG-GCT-TGC-TAG-AAC-TGA-A (SEQ ID NO: 5) as sense and 5′AAG-ACG-GTT-GTA-ACT-CCA-GAG-G (SEQ ID NO: 6) as antisense primers for sPLA₂-V to give a 329 bp; 5′CGC-GCC-CGG-CCA-AAT-AAA-ATA-A (SEQ ID NO: 7) as sense and 5′CAG-CGA-CGG-CAG-TAG-CAG-GAG-CAG (SEQ ID NO: 8) as antisense primers for sPLA₂-X to give a 410 bp; 5′CAG-AAG-AAA-GGC-AGT-TCT-GGA-TTG (SEQ ID NO: 9) as sense and 5′AAA-GCC-ACA-TCC-TGG-CTC-TGA-TT (SEQ ID NO: 10) as antisense for sPLA₂ receptor to give a 565 bp. The products were separated on 1.5% agarose gels.

### Determination of cPLA and its Phosphorylation

Cells (150,000) were plated on a 6-well plate. Twenty-four hours later, the culture medium was changed to a serum-free medium containing various treatments (intact or denatured porcine pancreatic PLA₂ (10 u/ml) with/without HyPE (10 M)). After incubation for 15 min, the cells were washed with cold PBS and lysed in 20 mM Tris-HCl (pH 7.5), 150 mM NaCl, 1 mM EDTA, 1 mM EGTA, 1% Triton X-100, 2.5 mM sodium pyrophosphate, 1 mM beta-Glycerophosphate, 1 mM sodium orthovanadate, 1 microgram/ml Leupeptin and 1 mM phenylmethylsulfonyl fluoride. Forty microgram of protein of each sample, under reducing conditions, were loaded on 8% SDS-PAGE. After electrophoresis, the proteins were transferred to PVDF membrane, Immobilone™-P (Millipore, USA). The blots were probed with the rabbit polyclonal phospho-cPLA₂ (Ser505) antibody (Cell Signaling technology, Inc., USA). Apparent molecular weight of the enzyme was 110 kDa. The membranes were probed with the respective antibodies overnight, followed by incubation with peroxidase-conjugated AffiniPure goat anti-rabbit IgG (1:5,000 dilution) (Jackson ImmunoResearch, West Grove, Pa.) for 1 h, and visualized using the ECL Western blot system (Pierce, Rockford, Ill.). Membranes were stripped, blocked, and then probed again with anti-cPLA₂ antibody (Cell Signaling technology, Inc., USA). The bands were scanned (Epson Perfection 3200 Photo), and the intensity was determined with the NIH image 1.63 software. All values are expressed in terms of untreated HT-1080 cells normalized to 1000%.

### Statistics

Statistical analysis was performed by student's t test and by Dunn test using ANOVA program.

### Example 1

### ExPLI (Lipid Conjugates) Effects on Cancer Cell Invasiveness

The effect of an extracellular cell-impermeable PLA₂ inhibitor (ExPLI) on the invasion of HT-1080 fibrosarcoma cells through a reconstituted basement membrane was

| | HyPE | HyDMPE | HepPE | HemPE | HemPE |
|---|---|---|---|---|---|
| | (MK-662) | (MK-677) | (MK-610) | (MK-545) | (MK-545) |
| | MMP-9 | MMP-9 | MMP-9 | MMP-9 | MMP-2 |
| 6 h (μM) | 0.2 | 0.4 | 0.75 | 1.0 | 1.5 |
| 24 h (μM) | 0.2 | 0.4 | 0.6 | >2.0 | 1.0 |

examined. HT-1080 cells were incubated for 24 h with HyPE, then washed, and challenged to cross through a Matrigel layer coated filter in a Boyden chamber.

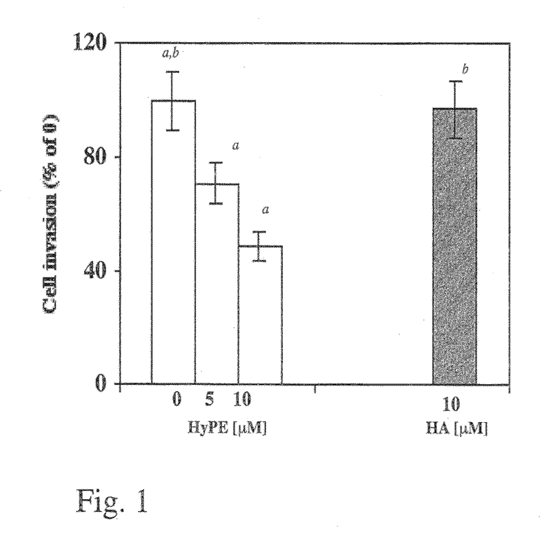

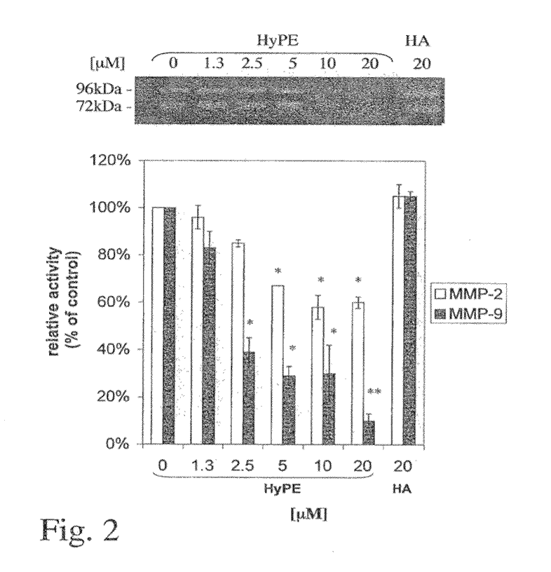

FIG. 1 demonstrates that pre-treatment of the cancer cells with HyPE effectively inhibited cell invasiveness without affecting cell viability or motility (not shown). It should be emphasized that cells were treated with HyPE prior to interaction with Matrigel and no ExPLI (lipid conjugates) was added during the invasion assay. In addition, as shown in FIG. 1, hyaluronic acid (HA) alone (without the lipidic portion of the ExPLI) did not affect cell invasiveness, demonstrating that the reduced invasiveness of cells after HyPE treatment is not due to ExPLI-exerted steric hindrance between the cells and the Matrigel.

Since the invasion of the basement membrane is dependent on the presence of collagen type IV degrading enzymes, the effect of HyPE effect on MMP-2 and MMP-9 secretion by the tumor cells was evaluated. Culture medium of HyPE-treated HT-1080 cells was collected and its collagenolytic activity was determined. FIG. 2 shows that the collagenolytic activity of both enzymes in the medium of HyPE-treated cells was reduced as a function of PLA₂ inhibitor concentration. Here too, treatment of cells with the GAG moiety alone did not inhibit MMP production.

The following Table demonstrate ED (concentration that exert 50% inhibition) of production of MMP-9 and MMP-2 by Human Fibrosarcoma (HT-1080) cells.

### Example 2

### ExPLI (Lipid Conjugates) Effects on PLA₂ Activity

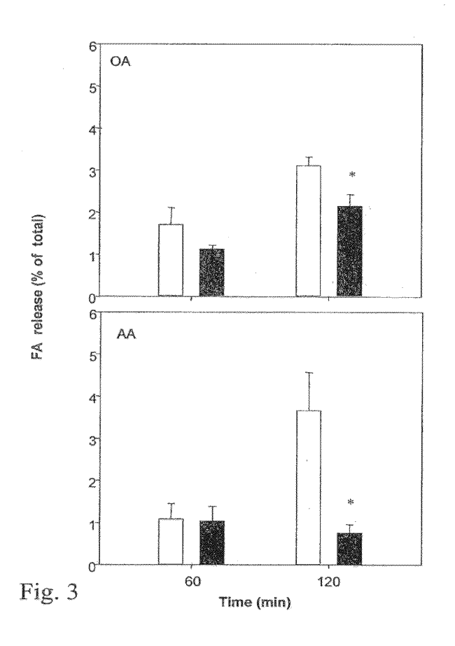

The direct effect of HyPE on PLA₂ activity in HT-1080 cells was determined. Since cPLA₂ is specific to AA-carrying phospholipids, while sPLA₂ has no fatty acid preference, the cell membrane phospholipids were metabolically pre-labeled with either radioactively-labeled AA or oleic acid (OA), and the temporal fatty acid secretion to the culture medium was determined. FIG. 3 demonstrates that treatment of HT-1080 cells with an ExPLI (lipid conjugates) inhibited the release of both AA and OA.

These findings together suggest that both sPLA₂ and cPLA₂ are involved in these processes, but since both activities are inhibited by the cell-impermeable inhibitor, it appears that they are controlled by sPLA₂.

Examination of the time course of the fatty acid release depicted in FIG. 3 shows that at 1 h, OA production, catalyzed by sPLA₂, is higher than that of AA, while the reverse is observed at 2 h. In addition, AA production is significantly enhanced at 2 h, while that of OA is relatively higher at 1 h. Moreover, at both time points, treatment with sPLA₂ inhibitor suppressed AA production to the level of the control, untreated cells. This may suggest that in HT-1080 cells the activity of sPLA₂ (producing both OA and AA) precedes that of cPLA₂ (producing only AA), and raises the possibility that cPLA₂ is activated subsequent to sPLA₂ action.

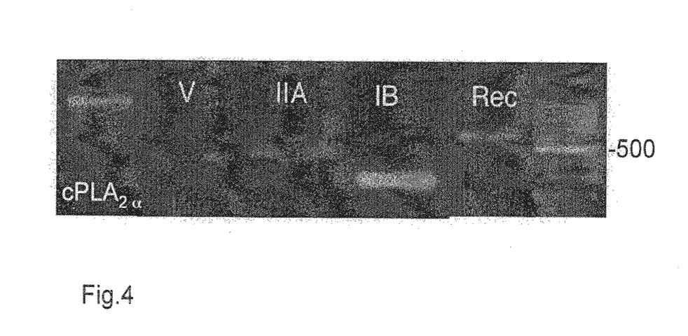

As noted above, sPLA₂ may act as a lipolytic enzyme and/or as a receptor ligand. RT-PCR was used to determine sPLA₂ types that are expressed in the HT-1080 cells. Two receptor-ligand sPLA₂s reported to act via M-type receptors, specifically IB and X, and two sPLA₂s that act mainly as lipolytic enzymes, specifically HA and V were investigated. Human HT-1080 fibrosarcoma cells express sPLA₂-IB, sPLA₂-IIA and sPLA₂-V as shown in FIG. 4. The cytosolic cPLA₂-IV alpha was identified as well. FIG. 4 also shows that HT-1080 cells express the receptor to sPLA₂-IB, thus implying the presence of all the components required for a PLA₂-mediated cell signaling.

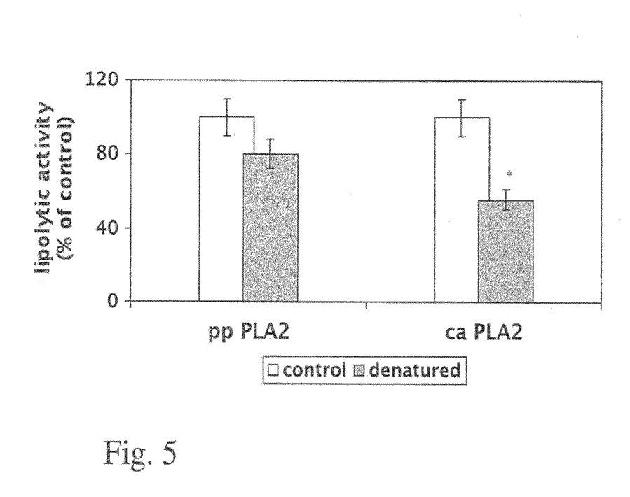

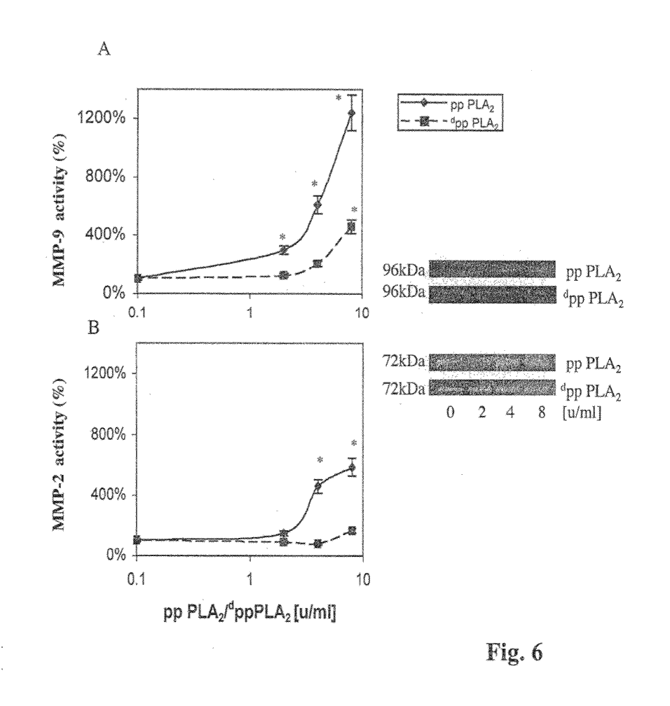

Exogenous sPLA₂ may act as a lipolytic enzyme, hydrolyzing cell membrane phospholipids, and also as receptor ligand, independent of its lipolytic activity. Both these activities may lead to cPLA₂ activation, as sPLA₂-produced lyso-phospholipids and receptor-mediated cell signaling lead to cPLA₂ phosphorylation, which is required for its activation. To differentiate between the two potential mechanisms for the activation of MMP production, exogenous sPLA₂s were subjected to boiling, which is expected to inactivate their lipolytic activity, and MMP production by HT-1080 was determined following treatment with the native and boiled sPLA₂s. Two commercially-available sPLA₂s were employed; porcine pancreatic (Type-IB), for which HT-1080 cells express a receptor, and Crotalos atrox (Type-HA) for which HT-1080 cells has no receptor. As shown in FIG. 5, boiling considerably suppressed the lipolytic activity of Type-IIA PLA₂, but had a small inhibitory effect on that of Type-IB (about 20%). On the other hand, production of both MMP-2 and MMP-9 was elevated by Type-IB PLA₂, in a concentration-dependent manner, as shown in FIG. 6. However, heating impaired the enzyme-receptor recognition, as heat-inactivation of sPLA₂-IB considerably suppressed its capacity to induce MMP production (to a much larger extent than the boiling effect on its lipolytic activity). MMP production was not affected by sPLA₂-IIA (not shown), nor was it attenuated by its heat inactivation, which inhibited its lipolytic activity (FIG. 5). These findings suggest that the induction of MMP production by sPLA₂ is mainly by a receptor-mediated process, rather than phospholipid hydrolysis-dependent

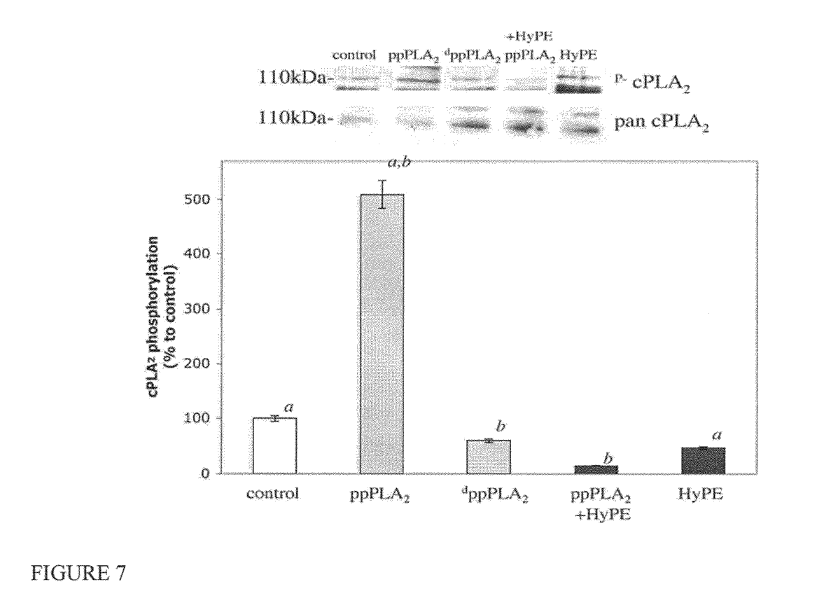

The present study supports findings that AA-derived eicosanoids are required for MMP production by the concomitant production of MMP and AA and inhibition of MMP secretion by the ExPLI (FIGS. 2 and 3). Since sPLA₂-dependent lipolysis does not contribute significantly to MMP production, one would assume that the required AA is provided by cPLA₂. This enzyme can be activated by phosphorylation that is induced by sPLA₂ receptor-mediated signaling, as has been previously reported for IB-sPLA₂. To examine this possibility in the present system, the phosphorylation status of cPLA₂ by native and heat-inactivated types IB and IIA sPLA₂ was assessed, and its inhibition by ExPLI (lipid conjugates). As shown in FIG. 7, sPLA₂-IB strongly enhanced cPLA₂ phosphorylation, and this was reduced to the basal level by heat inactivation of the enzyme or treatment with ExPLI (lipid conjugates). At the same time, sPLA₂-IA did not lead to any cPLA₂ phosphorylation (not shown).

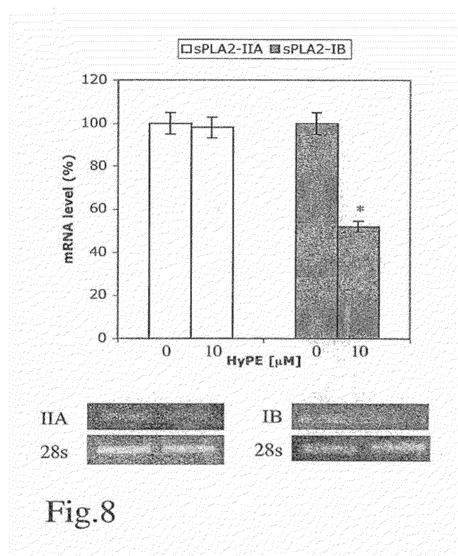

To further elaborate on the specific involvement of IB-PLA₂ in induction of MMP production, the ExPLI (lipid conjugates) effect on PLA2 mRNA expression was determined, using RT-PCR. As shown in FIG. 8, treatment of HT-1080 cells with ExPLI (lipid conjugates) had no effect of IIA-PLA₂ expression, but considerably reduced (by 50%) the expression of PLA₂-IB, concomitantly with the above shown inhibition of cell invasiveness (FIG. 1), MMP production (FIG. 2) and cPLA₂ phosphorylation (FIG. 6).

### Example 3

### Invasive Cellular Proliferative Disorders

The process of cancer spread entails multiple events, each of these is a worthy target for inhibitory drug action, including the rate of cell-proliferation, the rate of spread through blood vessels, the rate of invasiveness through contiguous and non-contiguous (metastases) tissues, and the rate of production of new blood vessels to supply the cancerous growth. Cancer cells frequently produce intracellular matrix tissue degrading enzymes which serve to enhance their invasive potential. Cancer is thus a multiphasic disease involving the process of tissue invasiveness, spread through tissue channels, angiogenesis and tumor vascularization. These latter processes depend upon the rates of proliferation of endothelial cells and smooth muscle cells.

Lipid-conjugates inhibit the production and activities of enzyme that break the basal membrane and enable the invasion of cancer cells, such as collagenase (metaloproteinase=MMP), heparinase and hyaluronidase:

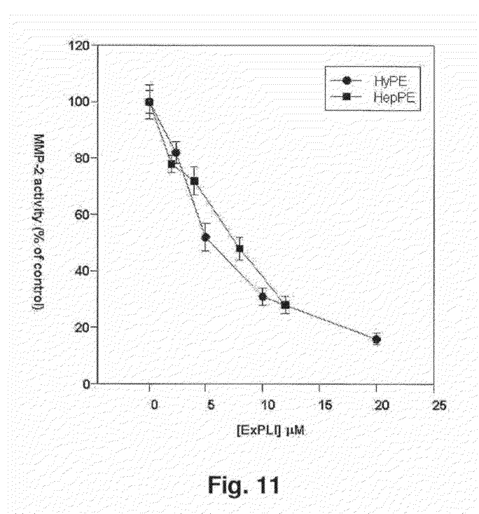

To demonstrate the Lipid-conjugate effect on collagenase, HT-1080 (fibrosarcoma) cells were incubated for 24 h with HYPE at the indicated concentration. The culture medium was then collected and its collagenase activity was determined by a zymographic assay. Each datum is average of two plates (FIG. 11).

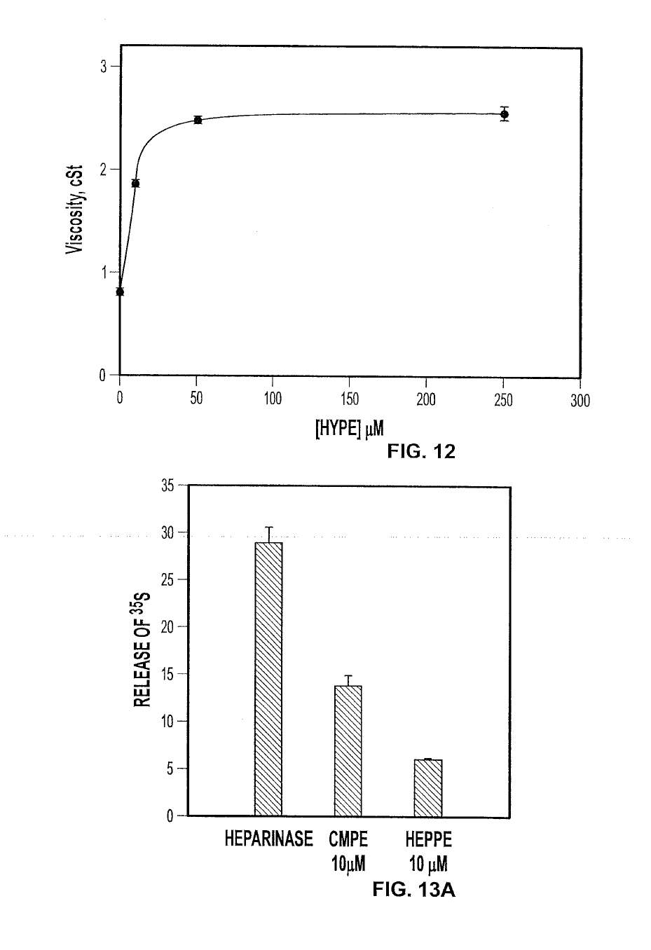

To demonstrate the ability of the Lipid-conjugates to inhibit hyaluronidase activity, hyaluronic acid (HA) in PBS (0.75 mg/ml) was interacted with hyaluronidase (15 U/ml) in the absence or presence of HYPE, at the indicated concentration for 1 h. HA degradation was determined by the change in the viscosity of its solution (FIG. 12).

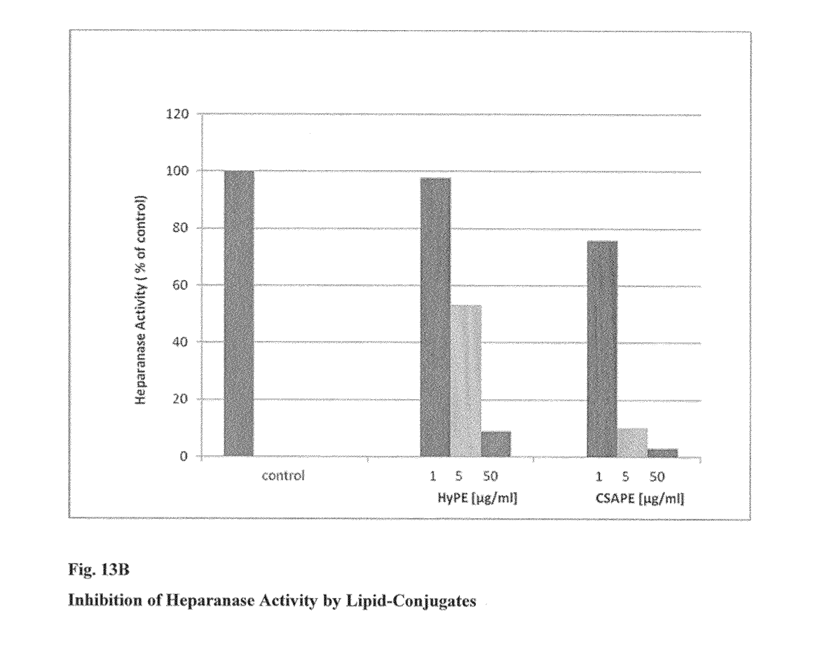

To demonstrate the inhibition of heparinase activity by Lipid-conjugates, BGM cells were incubated overnight with 50 μCi ³⁵SO₄²⁻ per well (to label the cell surface glycosaminoglycans). The cells then were washed 3 times with PBS before treating with 5 units of heparinase I in 200 μl PBS for 3 h. The medium was collected and its ³⁵S content was counted (FIG. 13). Recombinant heparanase enzyme, in the absence or presence of the lipid conjugates HyPE -or CSAPE) was incubated for 16 h (37° C., pH 6.2) on dishes coated with sulfate-labeled ECM, prepared as described (Vlodavsky et al., Cancer Res 43:2704-2711, 1983). Sulfate-labeled material released into the incubation medium was analyzed by gel filtration on a Sepharose 6B column. Nearly intact heparan sulfate proteoglycans are eluted just after the void volume (peak I, Kav<0.2, fractions 1-10) and heparan sulfate degradation fragments are eluted later with 0.5<Kav<0.8 (peak II, fractions 15-35). These fragments were shown to be degradation products of HS as they were 5-6 fold smaller than intact HS side chains, resistant to further digestion with papain and chondroitinase ABC, and susceptible to deamination by nitrous acid.

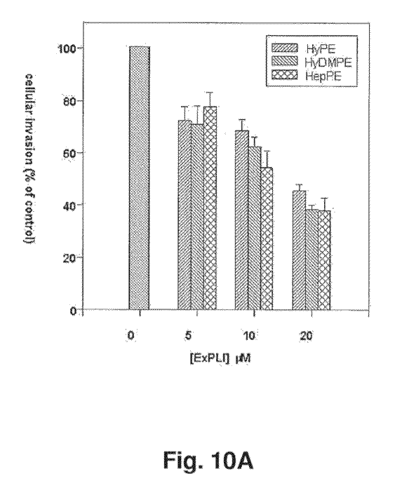

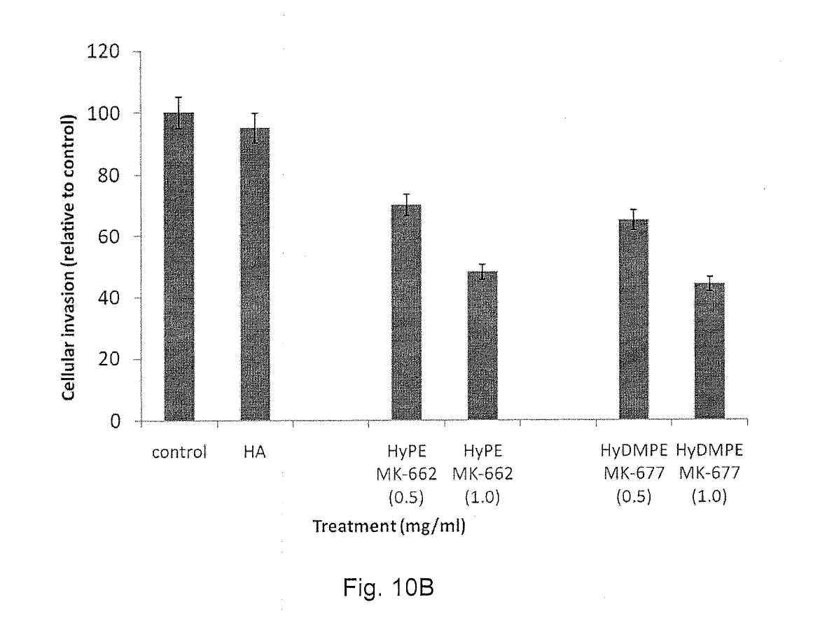

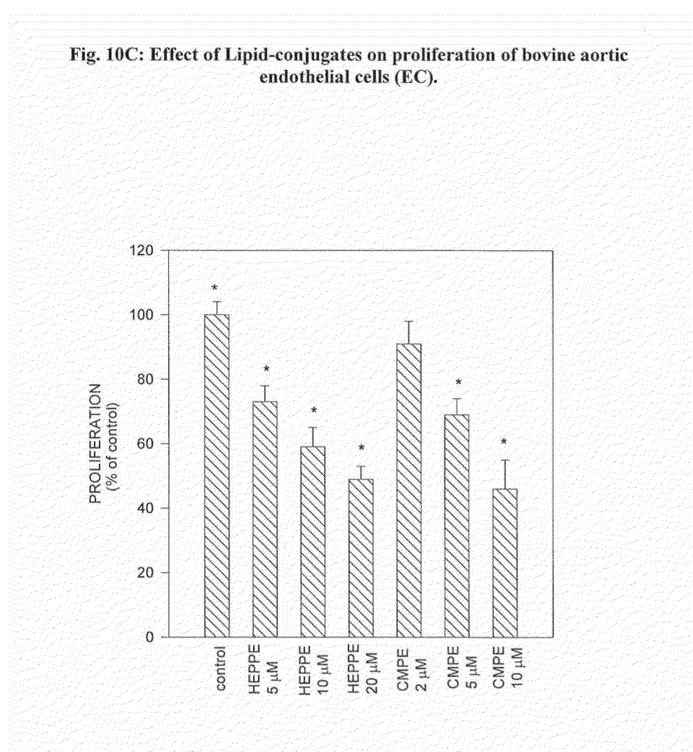

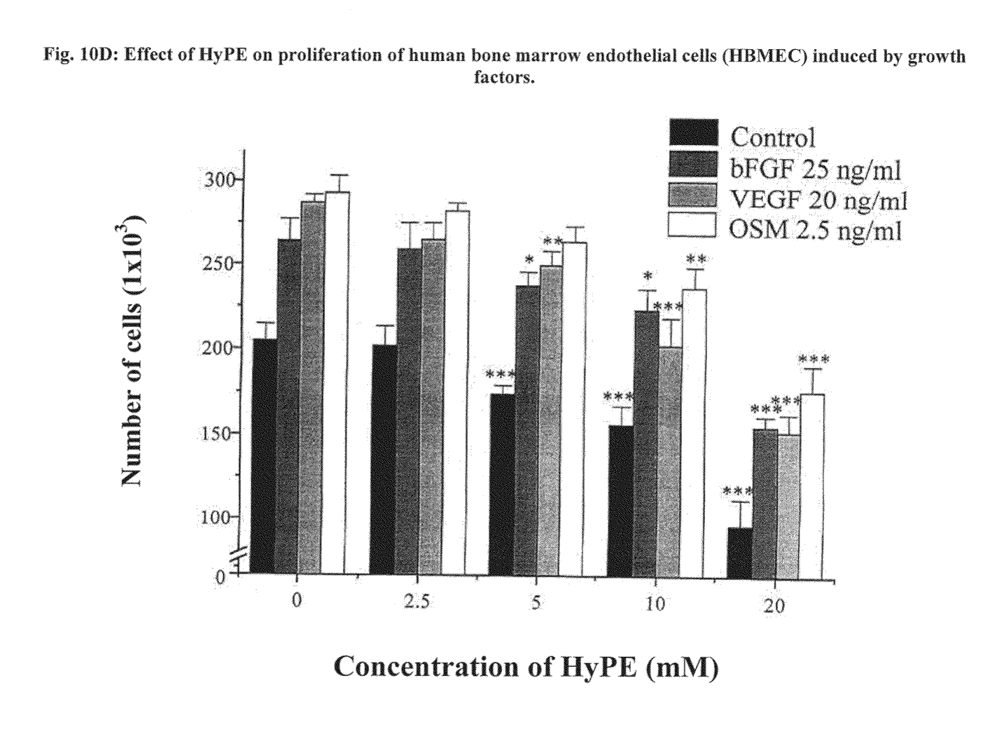

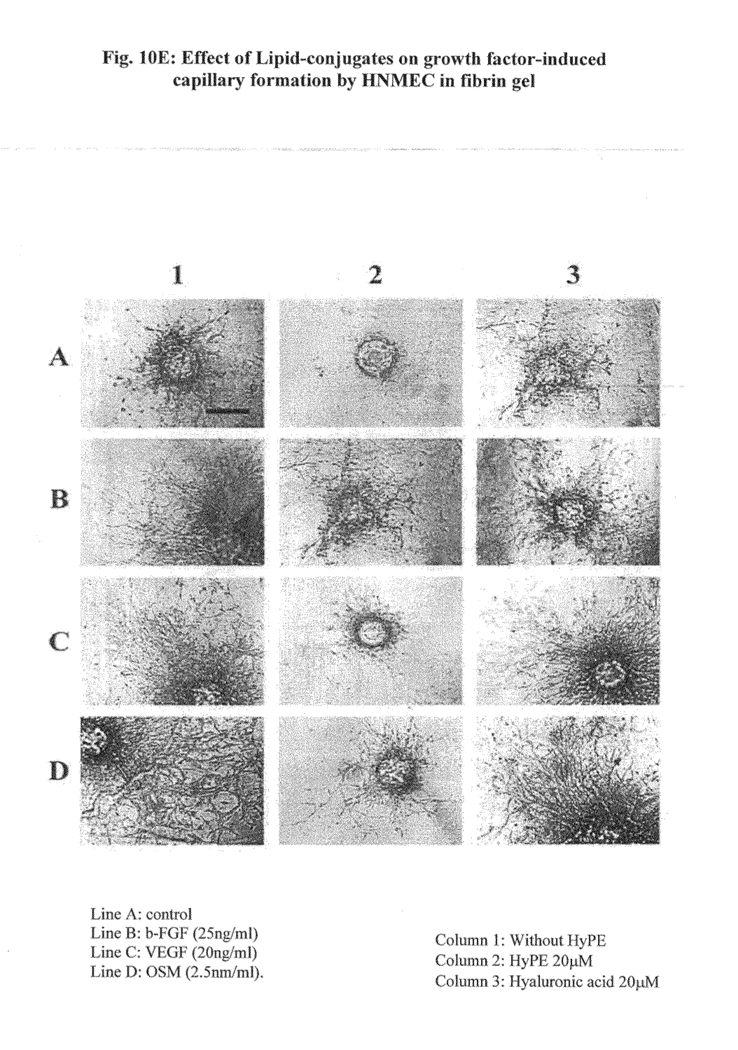

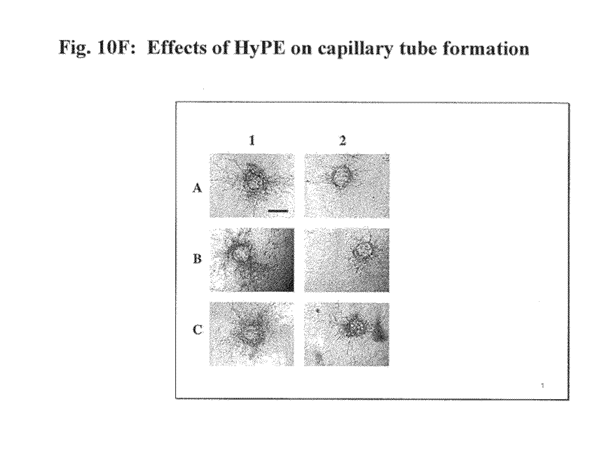

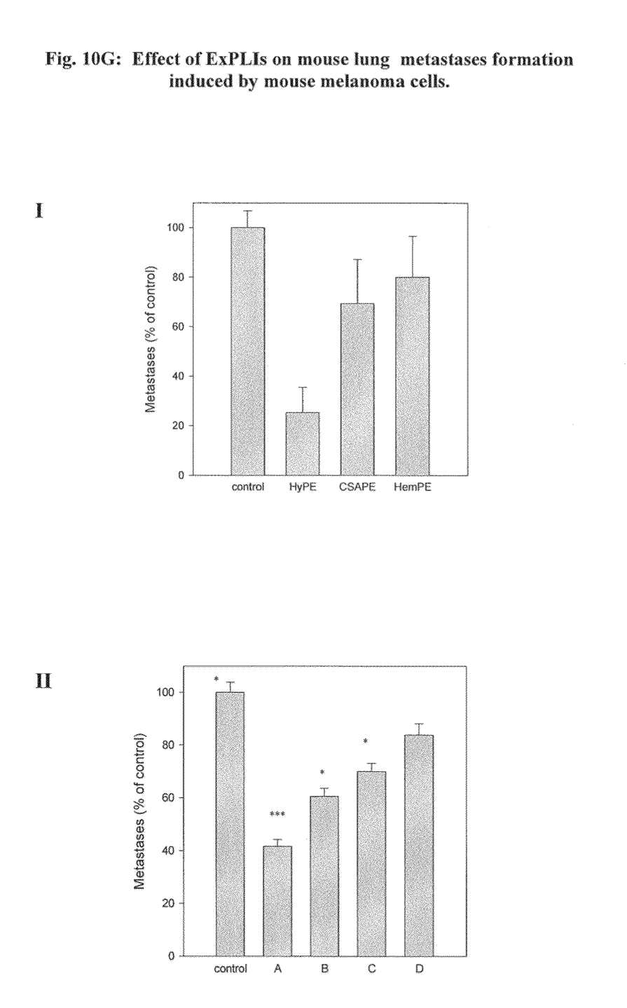

For showing the ability of the Lipid-conjugates to inhibit the invasion of tumor cells through basement membrane, the chemoattractant invasion assay was used: Polycarbonate fibers, 8 μm pore size, were coated with 25 μg of a mixture of basement membrane components (Matrigel) and placed in modified Boyden chambers. The cells (2×10⁵) were released from their culture dishes by a short exposure to EDTA (1 mM), centrifuged, re-suspended in 0.1% BSA/DMEM, and placed in the upper compartment of the Boyden chamber. Fibroblast conditioned medium was placed in the lower compartment as a source of chemoattractants. After incubation for 6 h at 37 C, the cells on the lower surface of the filter were stained with Diff-Quick (American Scientific Products) and were quantitated with an image analyzer (Optomax V) attached to an Olympus CK2 microscope. The data are expressed relative to the area occupied by untreated cells on the lower surface of the filter. (Albini et al., A Rapid In Vitro Assay for Quantitating the Invasive Potential of Tumor Cells. Cancer Res. 47:3239-3245, 1987). FIG. 10A demonstrates the Lipid-conjugate ability to attenuate cancer cell invasiveness.

Further experiments utilizing a Boyden chamber for chemo-invasion assays were performed: Matrigel (25 ug) was dried on a polycarbonate filter (PVP-free, Nucleopore). Fibroblast-conditioned medium (obtained from confluent NIH-3T3 cells cultured in serum-free DMEM) was used as the chemo-attractant. HT-1080 human fibrosarcoma cells were harvested (by brief exposure to 1 mM EDTA), washed with DMEM containing 0.1% bovine serum albumin, and added to the Boyden chamber (200k cells). The chambers were incubated in a humidified incubator at 37° C. (5% CO₂ 95% air) for 6 h. The cells that have traversed the Matrigel layer and attached to the lower surface of the filter were stained with Diff Quick (American Scientific Products) and counted. The results presented in FIG. 10B clearly demonstrated the inhibitory effect of dipalmitoyl phosphatidylethanolamine hyaluronic acid (HyPE) and dimyristoyl phosphatidylethanolamine hyurolonic acid (HyDMPE) indicate the actual compounds (FIG. 10).

For demonstrating Lipid-conjugate effect on proliferation of endothelial cells, bovine aortic endothelial cells were plated in culture dishes for 6 h, then washed to remove unattached cells. The remaining attached cells were incubated in the absence (control) or presence of Lipid-conjugates at the indicated concentration, and stimulated with VEGF (vascular endothelial growth factor) for 48 h. The cells were then washed, collected by trypsinization and counted in a Coulter counter. The results are mean±S.D. for 3 replications. *p<0.005 (FIG. 10C).

Similar effect was observed with human bone marrow microvascular endothelial cells (UBMEC), stimulated with different growth factors, namely VEGF, b-FGF (fibroblast growth factor), or OSM (oncostatin), as shown in FIG. 10D.

The capacity of the lipid-conjugates to control angiogenesis is illustrated in FIG. 10E. This Figure demonstrates the inhibitory effect induced by HyPE on capillary tube formation by HBMEC, in a three-dimensional fibrin gel, stimulated by the above growth factors. HyPE (20 μM) or hyaluronic acid (the carrier without the lipid moiety) were added to the HBMEC-coated beads in the fibrin simultaneously with the growth factors. Line A: control, Line B: b-FGF (25 ng/ml), Line C: VEGF (20 ng/ml), Line D: OSM (2.5 nm/ml). Column 1: Without HyPE, Column 2: HyPE 20 μM, Column 3: Hyaluronic acid 20 μM.

This raises the possibility that the observed inhibitory effect might be due to interference of the polymeric carrier with the accessibility of the growth factors to the cell surface. To examine this possibility, HBMEC cultured on the microcarrier beads were first stimulated with the growth factors for 3 h (to allow interaction with their receptors at the cell surface), then washed to remove the unbound growth factors and introduced into HyPE-containing fibrin matrix. As shown in FIG. 10F, under these conditions, capillary tube formation was effectively suppressed by HyPE, suggesting that the HyPE effect is not due to a defective growth factor accessibility due to steric hindrance by the polymer at the cell surface of the endothelial cells. Line A: b-FGF (25 ng/ml), Line B: VEGF (20 ng/ml), Line C: OSM (2.5 nm/ml). Column 1: Without HyPE, Column 2: HyPE 20 μM.

HyPE inhibits bFGF-, VEGF- and OSM-stimulated Capillary Tube Formation in a three-dimensional fibrin Gel. The corresponding quantitation of the capillary formation is presented in the following Table:

| | Length (μm) | | Width (μm) | |
|---|---|---|---|---|
| Treatment | −HyPE | +HyPE | −HyPE | +HyPE |
| Control | 232.23 ± 56.13 | 80.31 ± 30.59*** | 9.42 ± 1.65 | 8.32 ± 1.47 |
| BFGF | 533.92 ± 65.02 | 266.73 ± 23.17*** | 15.83 ± 2.96 | 11.21 ± 1.52* |
| VEGF | 511.09 ± 72.05 | 215.68 ± 31.22*** | 14.86 ± 1.46 | 9.32 ± 1.18** |
| OSM | 518.82 ± 58.49 | 234.85 ± 36.32*** | 16.89 ± 1.89 | 10.02 ± 1.00*** |

Each datum is mean ± SEM of 3 experiments; 5 beads were examined, in each field.
***p < 0.005,
**p < 0.01,
*p < 0.05

Bovine aortic endothelial cells were seeded in the absence and presence of HyPE or HyDMPE at the indicated concentration, on a layer of Matrigel (in culture dishes), enabling 3-dimensional growth and formation of capillaries. The capillary length was determined, using image analysis program, after 5 hours. The following Table demonstration the inhibition of capillary formation (angiogenesis) by lipid conjugates. Data are expressed as % of control (untreated):

| Treatment | Capillary Length (%) |
|---|---|
| Control | 100 |
| HyPE, 5 μM | 67 |
| HyPE, 10 μM | 32 |
| HyDMPE, 5 μM | 75 |
| HyDMPE, 10 μM | 58 |

In addition, the anti-proliferative effects of the Lipid-conjugates on bovine aortic smooth muscle cells, unstimulated or stimulated by thrombin, and on the proliferation of human venous smooth muscle cells was demonstrated:

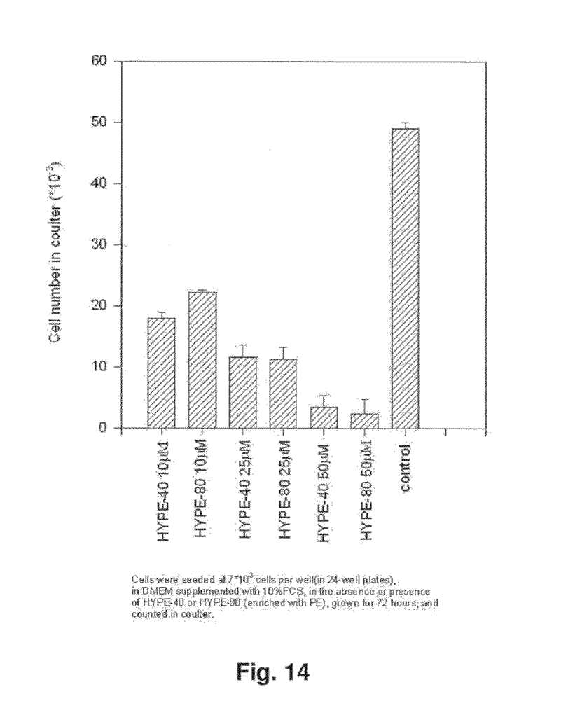

For unstimulated cells, bovine aortic smooth muscle cells were seeded at 7×10³ cells per well (in 24-well plates), in DMEM supplemented with 10% FCS, in the absence or presence of HYPE-40 or HYPE-80 (enriched with PE), grown for 72 h, and counted in Coulter (FIG. 14).

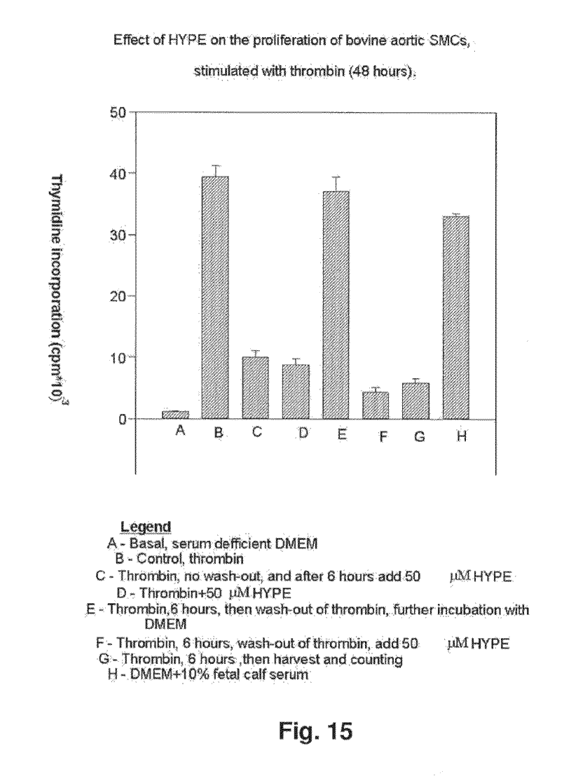

For stimulated cells, bovine aortic smooth muscle cells were grown under the conditions as above for 48 h, following pre-incubation for 6 h, as indicated, with either thrombin, fetal calf serum, Lipid-conjugate, or both. Cell growth is represented as the amount of thymidine incorporation (FIG. 15).

Smooth muscle cells (SMC) from human saphenous vein, were inoculated at 8×10′/cells/5 mm culture dish, in DMEM supplemented with 5% fetal calf serum and 5% human serum. A day later the cells were washed and incubated in the same culture medium in the absence (control) or presence of the Lipid-conjugate (HEPPE) or its polymeric carrier (heparin, at the same concentration as the HEPPE). After 5 days the cells were harvested (by trypsinization) and counted (FIG. 13). Each datum is mean±SEM for 3 replications (the same results were obtained in a second reproducible experiment). *p<0.005.

Effect of Lipid-conjugates on mouse lung metastases formation induced by mouse melanoma cells: 105 B16 F10 mouse melanoma cells were injected I.V. into a mouse (20-25 g). Three weeks later the lungs were collected and the metastases on the lung surface counted. The Lipid-conjugate effect, illustrated in FIG. 10G, was examined as follows: In experiment I, the indicated Lipid-conjugate (HyPE, CSAPE, HemPE) was injected I.P. (1 mg/mouse) 5 times a week for 3 weeks starting on day 1 (total of 15 injections) (FIG. 10G-I).

In FIG. 10G-II, HYPE (selected subsequently to experiment I) was injected I.P. (1 mg/mouse) as follows: A. 5 times a week for 3 weeks starting on day 1 (total of 15 injections); B. 5 times a week for 2 weeks starting from week 2 (total of 10 injections); C. One injection (I.P.) simultaneously with I.V. injection of the melanoma cells. D=Mice injected (I.P.) with hyaluronic acid alone (without PE), 5 times a week for 3 weeks, starting on day 1 (total of 15 injections). Each group included 6 mice. *p<0.0001, **p<1.10-5, ***p<2.10-7. The results clearly demonstrate that the Lipid conjugates inhibit melanoma-induced lung metastases.

These results support the notion that the Lipid-conjugates control the proliferation of smooth muscle cells, which is essential for tumor vascularization subsequent to capillary formation by endothelial cells.

Taken together, the experiments described above, demonstrate that administration of the Lipid-conjugates are effective therapy in the treatment of cancer growth and metastasis, by a plurality of mechanisms, including suppression of cell proliferation, invasion of cancer cells, angiogenesis and metastasis formation and tumor vascularization.

Thus, Lipid-conjugates are effective therapy for cellular proliferative disorders, such as cancer. The process of cancer spread entails multiple events, each of these is a worthy target for inhibitory drug action, including the rate of cell-proliferation, the rate of spread through blood vessels, the rate of invasiveness through contiguous and non-contiguous (metastases) tissues, and the rate of production of new blood vessels to supply the cancerous growth. Cancer cells frequently produce intracellular matrix tissue degrading enzymes which serve to enhance their invasive potential. Cancer is thus a multiphasic disease involving the process of tissue invasiveness, spread through tissue channels, angiogenesis and tumor vascularization. These latter processes depend upon the rates of proliferation of endothelial cells and smooth muscle cells.

### Example 4

### Conjugated Phosphatidylethanolamine (PE) inhibitors of Extracellular PLA2

Three types of sPLA₂s are expressed in HT-1080 cells: IB, IIA and V. These cells also express the M-type sPLA₂ receptor. These enzymes differ in their mode of action. IB exhibits low catalytic activity along with independent high affinity for M-type sPLA₂ receptor. The receptor-mediated signaling reportedly leads to activation of cPLA₂, which is a major source of cellular AA release. The IIA and V are structurally close heparin-binding isoforms participating in stimulus-induced AA release. In the present study we employed exogenous enzymes that represent the two sPLA₂ types, namely porcine pancreatic-derived (Type IB) and crotalos atrox venom-derived (Type IIA) forms, to differentiate between the lipolytic and receptor-mediated contributions to MMP production and cell invasiveness.

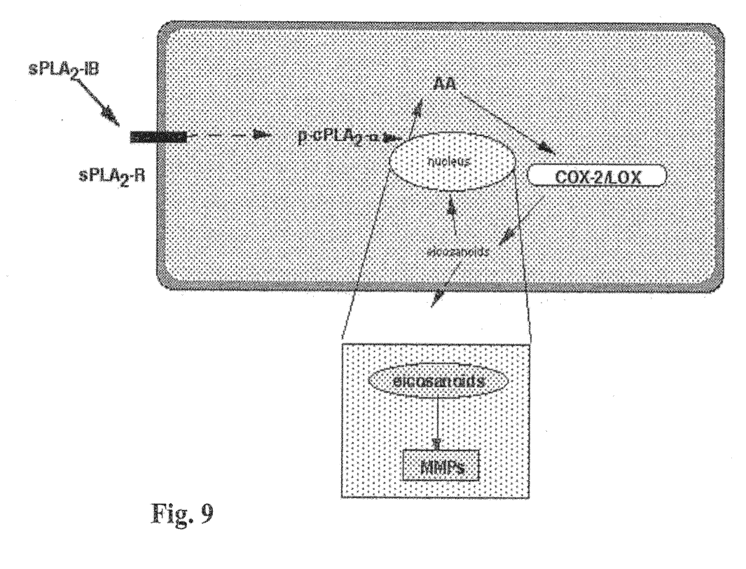

The results presented herein show that MMP-2/9 production by human fibrosarcoma HT-1080 cells and their invasiveness (FIGS. 1 and 2) correspond to AA production (FIG. 3), and these activities are concomitantly inhibited by the cell-impermeable sPLA₂ inhibitor (ExPLI). It further shows that sPLA₂-IB activates MMP production (FIG. 8) via a receptor-mediated process, rather than its lipolytic activity (FIGS. 5 and 6). Concomitantly, sPLA₂-IB activates cPLA₂ by its phosphorylation (FIG. 7), and intracellular cPLA₂ phosphorylation is induced by M-type sPLA₂ receptor interaction. All the above processes are inhibited by the ExPLI (lipid conjugates), thus assigning a pivotal role for sPLA2-IB in MMP activation and subsequent cancer cell invasiveness. Taken together, these findings suggest that sPLA₂-IB-mediated MMP activation is compatible with the sequence of events illustrated in FIG. 9: sPLA₂-IB secreted to the extracellular medium interacts with its membrane receptor (on its own and neighboring cells), signals the phosphorylation and subsequent activation of the cytosolic cPLA₂, which provides the AA for production of the eicosanoids required for MMP production/action.

Of specific interest is the finding that although sPLA₂-IB induces MMP production by acting as a receptor-ligand, rather by its lipolytic activity, its effect is suppressed by the ExPLI (lipid conjugates), which is designed to inhibit membrane phospholipids hydrolysis. Additionally, it was found that in parallel to inhibition of MMP production, the ExPLI (lipid conjugates) reduced the production of AA, which attributed to cPLA₂, and also OA, which is a product of sPLA₂ and other PLA (but not cPLA₂). It is thus possible that lipolytic activity of sPLA₂ and/or PLA₂ also take part in sPLA₂-IB-induced MMP production. The results indicate that activated cPLA₂ provides the AA for production of eicosanoids required for MMP activation/action.

### Example 5

### Toxicity Tests

The following compounds were tested: HyPE, CMPE, CSAPE and HepPE. The compounds were injected IP at one dose of 1000, 500 or 200 mg/Kg body weight. Toxicity was evaluated after one week, by mortality, body weight, hematocrit, blood count (red and white cells), and visual examination of internal organs after sacrifice. These were compared to control, untreated mice. Each dose was applied to a group of three mice. No significant change in the above criteria was induced by treatment with these compounds, except for the HepPE, which induced hemorrhage.

The non-toxicity of the Lipid conjugates is demonstrated in Table 6 and Table 7, depicting the results obtained for HyPE in acute (6) and long-term (7) toxicity tests.

TABLE 6
Acute toxicity

| Dose of | | | | | |
|---|---|---|---|---|---|
| HyPE | | | | | |
| (mg/kg | | | | | |
| body | | | RBC × | WBC × | Hematocrit |
| weight) | Body weight (g) | | 10⁶ | 10³ | % |
| 0.0 | 21.9 ± 0.2 | 22.6 ± 0.3 | 10.7 ± 0.4 | 9.3 ± 0.3 | 45.0 ± 0.5 |
| (control) | | | | | |
| 250 | 22.1 ± 0.4 | 23.1 ± 0.6 | 11.4 ± 0.1 | 7.7 ± 0.2 | 43.3 ± 0.7 |
| 500 | 21.4 ± 0.3 | 22.3 ± 0.4 | 11.5 ± 0.3 | 8.1 ± 1.3 | 44.7 ± 2.3 |
| 1000 | 21.7 ± 0.2 | 22.1 ± 0.2 | 10.9 ± 0.4 | 7.4 ± 0.6 | 40.3 ± 0.7 |

RBC = red blood cells. WBC = white blood cells. Each datum is mean ± SEM.

For long-term toxicity test of HyPE, a group of 6 mice received a dose of 100 mg HyPE/Kg body weight, injected IP 3 times a week for 30 weeks (total of 180 mg to a mouse of 20 g). Toxicity was evaluated as for Table 5. No mortality, and no significant change in the above criteria was induced by this treatment, compared to normal untreated mice (see Table 6), as depicted in Table 7.

TABLE 7
Results at week 30:

| | | RBC × | WBC × | Hematocrit |
|---|---|---|---|---|
| | Body weight (g) | 10⁶ | 10³ | % |
| Control | 39.5 ± 3.1 | 10.9 ± 0.8 | 9.3 ± 0.6 | 45.0 ± 0.8 |
| (untreated) rats | | | | |
| HyPE- | 39.0 ± 2.7 | 11.7 ± 0.7 | 8.1 ± 15 | 43.4 ± 4.9 |
| injected rats | | | | |

### Example 6

### Preparation of Hyaluronic Acid (HA) Solution

4 g of chlorocresol was dissolved in 4 L of deionized (DI) water (0.1% o solution). HA UL 15 was dissolved in 4 L of 0.1% chlorocresol solution with mechanical stirring. To prevent clogging of the ultrafiltration membranes, the HA solution was filtered through a 100 μm filter followed by a 50 μm filter followed by a 10 μm filter, all previously disinfected with 10% hydrogen peroxide and washed with copious amounts of DI water to ensure hydrogen peroxide has been removed (verified with peroxide-detecting strips).

### Example 7

### Ultrafiltration Fractionation of Hyaluronic Acid (HA)

HA solution of Example 6 was loaded into the Centramate system, previously disinfected with 10% hydrogen peroxide and washed with copious amounts of DI water to ensure hydrogen peroxide has been removed (verified with peroxide-detecting strips).

By means of constant volume diafiltration with 70 kDa Omega TFF membranes, 20 L of 0.1% chlorocresol solution, prepared as described in Example 6, was ultrafiltered, collecting the filtrate, the fraction less than 70 kDa, in a carboy, previously disinfected with 10% hydrogen peroxide. The pump speed and valves shall be set such that the retentate flow is ten times the filtrate flow and the feed pressure is less than 40 PSI.

The 70 kDa membranes were replaced with 30 kDa membranes and the Centramate system was disinfected with 10% hydrogen peroxide.

5 L of the filtrate, the fraction less than 70 kDa, were loaded into the reservoir and by means of constant volume diafiltration, the remaining 35 L in the carboys of the fraction less than 70 kDa were ultrafiltered. The reservoir volume was reduced to 2 L and an additional 10 L of DI water was ultrafiltered to remove the chlorocresol (confirmed by appropriate GC assay). The reservoir volume was further reduced to 1 L, reducing the pump speed, if necessary, to keep the feed pressure below 40 PSI. The reservoir was then emptied directly into an autoclaved lyoguard container, closed, frozen and lyophilized to yield HA UF 70/30. GPC analysis was performed to ensure that this lot of HA UF 70/30 was consistent with earlier batches. A bioburden assay and an appropriate GC assay for chlorocresol was performed. Karl Fischer analysis was performed to determine the water content of HA UF 70/30.

### Example 8

### HyPE Synthesis Reaction

24 g of 2-(N-morpholino)ethanesulfonic acid (MES) were dissolved in 125 mL of DI water and the pH was adjusted to pH 6.4 by addition of 4 N NaOH.

2.5 g of dipalmitoylphosphatidylethanolamine (DPPE) and 25 g of hydroxybenzotriazole (HOBT) were dissolved in 940 mL of tert-butanol and 80 mL of water with stirring and heating at 45° C. in a 12 L round bottom flask (forming a closed system with the pump and the sonciator, all of which will have been previously autoclaved and/or disinfected with 70% isopropanol). To this was added 850 mL of water and 115 mL of the MES solution. The pH of this solution was adjusted to pH 6.4 by addition of 2.5 N NaOH. 25 g of HA UF 70/30 of Example 7 were then dissolved with stirring and heating at 45° C. 25 g of 1-ethyl-3-(3-dimethylaminoethyl)carbodiimide (EDAC) were then added, the pump and the sonicator were turned on and the system was kept between 40 and 50° C. for 3 hours. GPC analysis was performed to monitor the progress of the reaction. After 3 hours the sonicator and the pump were turned off and the solution was stirred at room temperature overnight. The following day 750 mL of acetonitrile were added to precipitate HyPE. This was allowed to stand for 30 minutes after which the supernatant was removed. To this was added 7.5 L of 2% Na₂CO₃, previously prepared by dissolving 150 g of Na₂CO₃ in 7.5 L in DI water. Vigorous mechanical stirring for at least 2 hours hydrolyzed urea related byproducts. The solution was neutralized with 6 N HCl while the temperature was kept at 20-25° C. by passing the solution through a cooled, jacketed flow cell.

### Example 9

### Alkaline Ultrafiltration of HyPE

2.25 kg of NaHCO₃ was dissolved in 150 L of 0.1% chlorocresol solution, prepared by dissolving 150 g of chlorocresol in 150 L of DI water. By means of valves, the closed reaction system was diverted so that the digested, neutralized HyPE solution of Example 8 was pumped from the round bottom flask to the centrasette system. By means of constant volume diafiltration with a 10 kDa Omega TFF membrane, 150 L of 1.5% NaHCO₃ in 0.1% chlorocresol solution was ultrafiltered, discarding the filtrate, the fraction less than 10 kDa. The pump speed and valves were set such that the retentate flow was ten times the filtrate flow and the feed pressure was less than 40 PSI. GPC analysis was performed to ensure the disappearance of urea-related peaks at ˜13.2 min and the HOBT peak at ˜17.2 min. The solution was neutralized with 6 N HCl while the temperature was kept at 20-25° C. by passing the solution through a cooled, jacketed flow cell.

### Example 10

### Extraction of HyPE

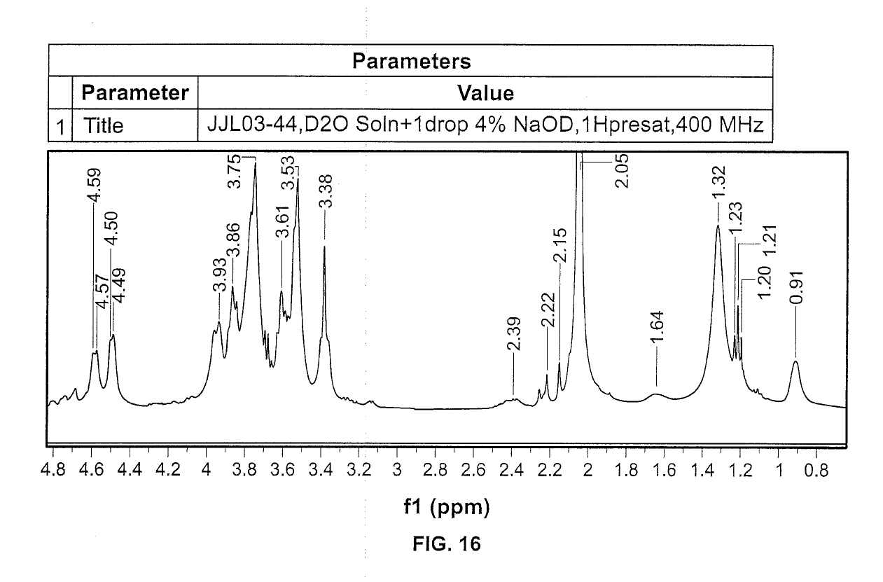

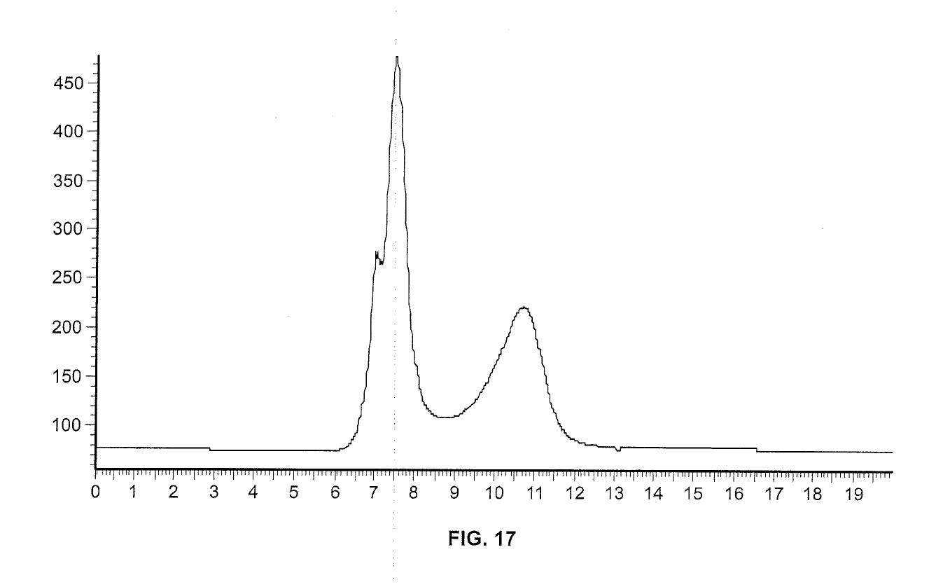

An extraction solution was made by mixing 3 L of dichloromethane, 3 L of ethanol and 2.25 L of methanol. 7.5 L of the extraction solution was added to a round bottom flask containing 3 L of crude HyPE solution of Example 9. This was stirred vigorously for 15 minutes after which time it was allowed to stand for 45 min. The lower dichloromethane layer was removed. By means of constant volume diafiltration the solution was washed with 100 L of DI water to remove the methanol and ethanol. GPC analysis was performed to ensure the disappearance of peaks at ˜14 min. The volume was reduced to 3 L and emptied directly into 2 autoclaved lyoguard containers, closed, frozen and lyophilized to yield HyPE. NMR and HPLC data for isolated HyPE are shown in FIG. 16 and FIG. 17.

### Example 11

### Preparation of Hype from 9.54 kD Hyaluronic Acid

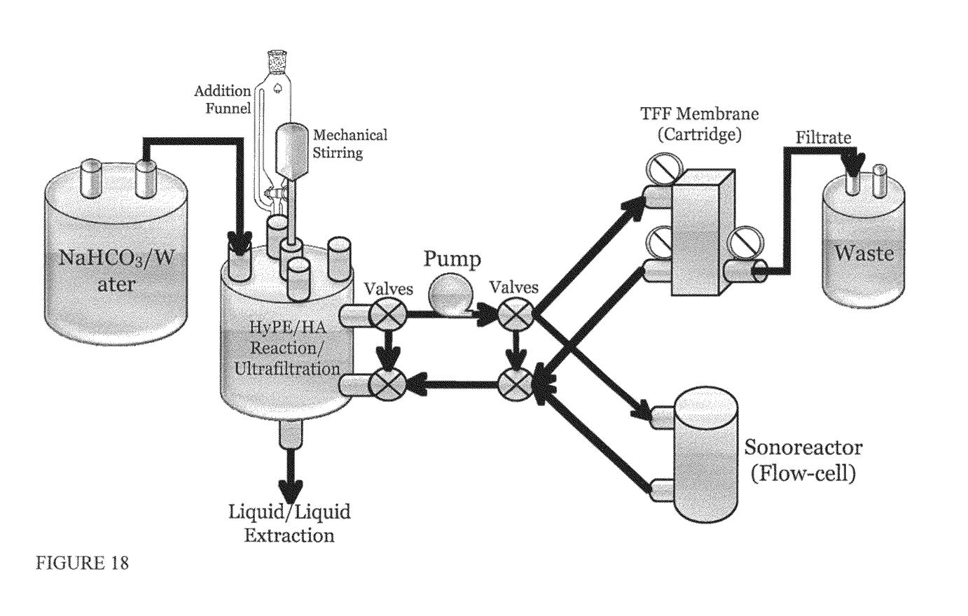

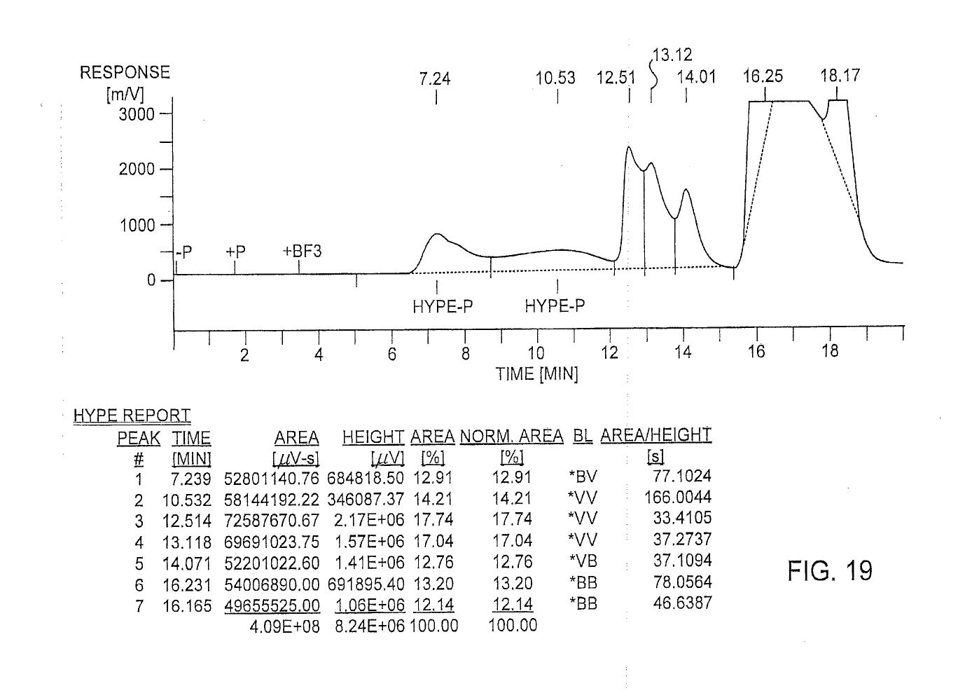

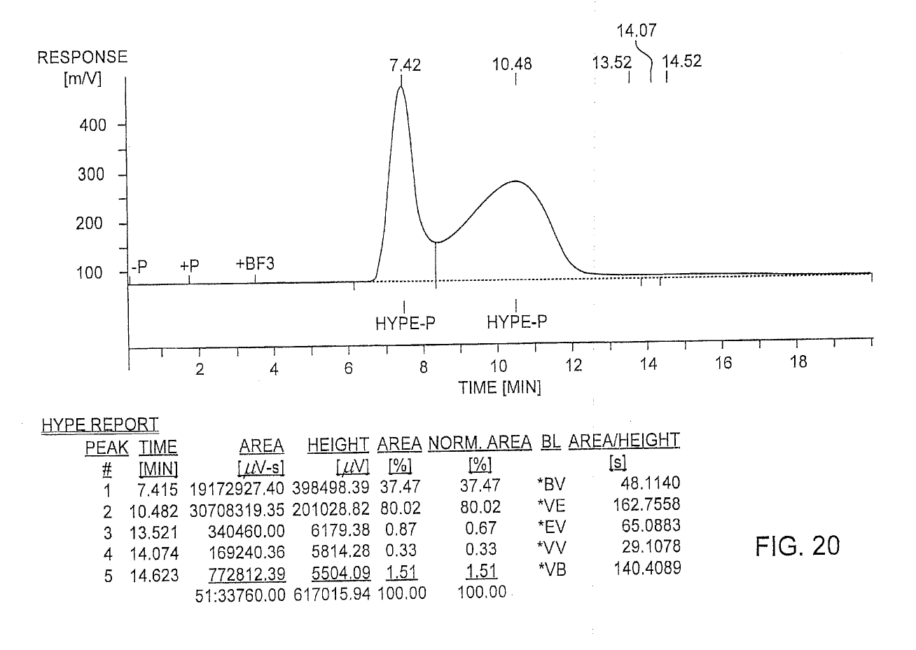

MES buffer was prepared by dissolving 14.5 g of MES in 75 mL of DI-H₂O and adjusting the pH to 6.4 with 4N NaOH. Using an apparatus similar to that depicted in FIG. 18, 10.0 g of HOBT was dissolved in 225 mL of DI-H₂O, 60 mL MES buffer, 12 mL of tert-butanol. The pH was adjusted to 6.4 with 4N NaOH. 15.1 g of HA was dissolved in 350 mL of DI-H₂O. 1.25 g or DPPE was dissolved in 440 mL of tert-butanol and 90 mL DI-H₂O with heating to 55 deg C. The solutions of HA and HOBT were warmed to 35 deg C. and mixed. The DPPE solution, at 50 deg C. was then added to afford a clear solution. This was allowed to cool to 43 deg C., when it was added to the flask and circulated through the sonoreactor system. Some component of the reaction mixture came out of solution and it was necessary to heat the reaction mixture to 49 deg C. with sonication to form a clear solution. 12.5 g of EDAC was added as a powder to the reaction mixture at a temperature of 45 deg C. Sonication began with a power of 180 watts. The reaction was monitored by GPC as shown in FIG. 19 (after 6 h) and because the extent of agglomeration, as observed by the ratio of the area of the first peak to that of the second continued to increase, the reaction was allowed to continue beyond the normal 3 h and was continued the next day. The sonication was turned off and the reaction mixture was filtered through a 0.45 μm filter to remove a small amount of rubber debris apparently from the stator. The solution (1200 mL) was extracted with 600 mL DCM and 600 mL MeOH. The resulting emulsion quickly resolved and the aqueous layer was extracted again with 500 mL DCM and 500 mL EtOH. Finally, the aqueous layer was extracted with 250 mL DCM and 250 mL EtOH and left over the weekend. Residual DCM was removed by rotovaporation at 35 deg C. and 200 Torr. The solution was then transferred to a previously cleaned centrasette ultrafiltration system with a 10 kDa membrane and by constant volume diafiltration was washed with 5 L of 1.5% NaHCO₃ to remove residual organic solvents. The pH was then increased by slow addition of 2% Na₂CO₃ to pH 9.2. The solution was stirred for 1 hour at room temperature. After further washing with 30 L of 1.5% NaHCO₃ the peat at −12.5 min had disappeared and the solution was washed with 30 L of DI-H₂O until pH 7. To remove any digestion/ultrafiltration byproducts, such as free palmitic acid, the solution was then extracted again with 1 L DCM, 1 L MeOH and 0.75 L EtOH. The aqueous layer was extracted again with 400 mL DCM and 50 mL EtOH and finally a third time with 400 mL DCM and 50 mL EtOH. Residual DCM was removed by rotovaporation at 30 deg C. and 200 Torr. By constant volume diafiltration residual MeOH and EtOH were removed by washing with 15 L DI-H₂O. The solution was concentrated to 1 L and filtered through a 0.2 μm filter into a lyoguard container and placed in the lypholizer. It was frozen by lowering the shelf temperature to −70 deg C. When frozen, vacuum was applied (14 mT) and the shelf temperature was raised to 30 deg C. Five days later 6.134 g of HyPE was recovered with a water-corrected weight of 5.2 g which corresponds to a 42% yield based on 12.5 g (water corrected) of HA. Total phosphorus was found to be 0.28% (dry basis). By LC/MS assay, 1,456 ppm of free EDU were found and after exposure to NaOH 12,557 ppm total EDU was found. No HOBT was detected and MES was less than 80 ppm. GPC of the final product is shown in FIG. 20.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What we claim is:

1. A method for treating a subject afflicted with lung cancer, comprising the step of administering to said subject a composition comprising a compound represented by the structure of the general formula (I): 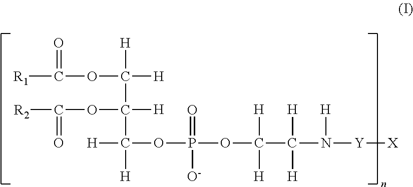
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan alginate or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

2. The method of claim 1, wherein said n is a number from 2 to 1,000.

3. The method of claim 1, wherein said glycosaminoglycan is selected from the group consisting of hyaluronic acid, heparin, heparan sulfate, chondrotin sulfate, keratan, keratan sulfate, dermatan sulfate or a derivative thereof.

4. The method of claim 1, wherein said phosphatidylethanolamine is a myristoyl or palmitoyl phosphatidylethanolamine.

5. The method of claim 1, wherein said phosphatidylethanolamine is a dipalmitoyl phosphatidylethanolamine, or dimyristoyl phosphatidylethanolamine.

6. A method for attenuating invasiveness of a cancer cell, comprising the step of subjecting said cancer cell to a composition comprising a compound represented by the structure of the general formula (I): 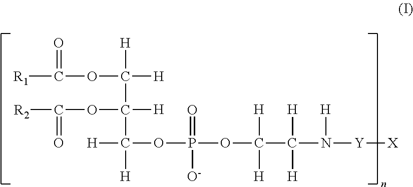
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan, alginate or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

7. The method of claim 6, wherein said n is a number from 2 to 1,000.

8. The method of claim 6, wherein said glycosaminoglycan is selected from the group consisting of hyaluronic acid, heparin, heparan sulfate, chondrotin sulfate, keratan, keratan sulfate, dermatan sulfate or a derivative thereof.

9. The method of claim 6, wherein said phosphatidylethanolamine is a myristoyl or palmitoyl phosphatidylethanolamine.

10. The method of claim 6, wherein said phosphatidylethanolamine is a dipalmitoyl phosphatidylethanolamine, or dimyristoyl phosphatidylethanolamine.

11. A method for inhibiting proliferation of an endothelial cell, comprising the step of subjecting said endothelial cell to a composition comprising a compound represented by the structure of the general formula (I): 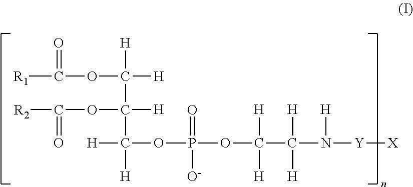
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan, alginate or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

12. The method of claim 11, wherein said n is a number from 2 to 1,000.

13. The method of claim 11, wherein said glycosaminoglycan is selected from the group consisting of hyaluronic acid, heparin, heparan sulfate, chondrotin sulfate, keratan, keratan sulfate, dermatan sulfate or a derivative thereof.

14. The method of claim 11, wherein said phosphatidylethanolamine is a myristoyl or palmitoyl phosphatidylethanolamine.

15. The method of claim 11, wherein said phosphatidylethanolamine is a dipalmitoyl phosphatidylethanolamine, or dimyristoyl phosphatidylethanolamine.

16. The method of claim 11, wherein capillary formation is inhibited by inhibiting proliferation of said endothelial cell.
